(19) United States
(12) Patent Application Publication
(10) Pub. No.: US 2026/0355246 A1
(43) Pub. Date: Nov. 12, 2026

(54) SYSTEMS TARGETING PSCA AND PSMA AND METHODS OF USE THEREOF

(71) Applicant: Arsenal Biosciences, Inc., South San Francisco, CA (US)

(72) Inventors: Joseph Choe, San Francisco, CA (US); Jianying Liu, Albany, CA (US); Sarah Lensch, Redwood City, CA (US); Nishant Mehta, San Mateo, CA (US); Hong Nan, Oakland, CA (US); Beatriz Millare, San Francisco, CA (US); Suchismita Mohanty, South San Francisco, CA (US); Alba Gonzalez, South San Francisco, CA (US); Hongruo Yun, South San Francisco, CA (US)

(21) Appl. No.: 19/666,039

(22) Filed: May 1, 2026

Related U.S. Application Data
(60) Provisional application No. 63/799,294, filed on May 2, 2025.
(60) Provisional application No. 63/851,228, filed on Jul. 25, 2025.

(51) Int. Cl. A61K 35/17; A61K 40/11; A61K 40/31; A61K 40/42; A61P 35/00; C07K 14/47; C07K 14/705; C07K 14/71; C07K 16/30; C12N 15/85

(52) U.S. Cl. CPC A61K 35/17; A61K 40/11; A61K 40/31; A61K 40/4276; A61P 35/00; C07K 14/4705; C07K 14/70578; C07K 14/71; C07K 16/3069; C12N 15/85; A61K 2239/13; A61K 2239/21; A61K 2239/29; C07K 2317/31; C07K 2317/565; C07K 2317/622

(57) ABSTRACT

Provided herein are antibodies and chimeric priming receptors that bind PSCA and antibodies and chimeric antigen receptors that bind PSMA. Also provided are systems of chimeric priming receptors that bind PSCA and chimeric antigen receptors that bind PSMA, cells expressing such systems, and methods of use thereof.

### CROSS REFERENCE TO RELATED APPLICATIONS

This application claims the benefit of U.S. Provisional Application No. 63/799,294, filed May 2, 2025, and U.S. Provisional Application No. 63/851,228, filed Jul. 25, 2025 both of which are hereby incorporated in their entirety by reference.

### SEQUENCE LISTING

The instant application contains a Sequence Listing which is hereby incorporated by reference in its entirety. Said XML copy, created on Apr. 24, 2026, is named ANB-229US_SL.xml, and is 99,451 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using CAR-T cells is promising because the modified T cells have the potential to recognize cancer cells in order to more effectively target and destroy them.

After the T cells are engineered with the CARs, the resulting CAR-T cells are introduced into patients to attack tumor cells. CAR-T cells can be either derived from T cells in a patient's own blood (autologous) or derived from the T cells of another healthy donor (allogeneic). Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing.

However, there remain some concerns and limitations to CAR T cell-based immunotherapy. Some CAR T cells may engage with normal cells expressing low levels of target antigens, leading to off target toxicity. Moreover, the tumor microenvironment (TME) of solid tumors is typically hostile to immune cells often limiting the efficacy of CAR T cell therapies. Finally, CAR T cell therapy of solid tumors have been limited by the occurrence of the CAR T cells eventually displaying an exhausted phenotype. Thus, additional therapies that provide an efficacious and prolonged response while having reduced off-target toxicity remain desirable.

### SUMMARY

Described herein are logic-gated immunotherapies, e.g., CAR T cells, that are highly dependent on the presence of two tumor antigens for activity (fidelity), demonstrate a potent and prolonged response against cells expressing the tumor antigens (efficacy) to overcome the immunosuppressive mechanisms of the tumor microenvironment (TME), and have reduction of or resistance to an exhaustion phenotype.

In one aspect, provided herein is a system comprising:
a. a first chimeric polypeptide comprises a priming receptor comprising a first antigen-binding domain that specifically binds Prostate Stem Cell Antigen (PSCA) (SEQ ID NO: 1);
b. a second chimeric polypeptide comprising a chimeric antigen receptor (CAR) comprising a second antigen-binding domain that specifically binds to Prostate-Specific Membrane Antigen (PSMA) (SEQ ID NO: 2);
c. an optional third chimeric polypeptide comprising a synthetic pathway activator (SPA); and
d. at least one or more nucleic acid(s) comprising at least two nucleic acid sequences at least 15 nucleotides in length complementary to:
   i. a nucleic acid encoding human Fas Cell Surface Death Receptor (FAS) comprising the sequence set forth in SEQ ID NO: 3; and
   ii. a nucleic acid encoding human Transforming Growth factor (TGF)-B Receptor 2 (TGFBR2) comprising the sequence set forth in SEQ ID NO: 4.

In an embodiment, the first antigen-binding domain comprises a first variable heavy (VH) chain sequence comprising three heavy chain CDR sequences, CDR-H1, CDR-H2, and CDR-H3, of the VH sequences set forth in SEQ ID NO: 45, and a first variable light (VL) chain sequence comprising three light chain CDR sequences, CDR-L1, CDR-L2, and CDR-L3, of the VL sequences set forth in SEQ ID NO: 46, optionally wherein:
a. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52.

In an embodiment, the second antigen-binding domain comprises a second variable heavy (VH) chain sequence comprising three heavy chain CDR sequences, CDR-H1, CDR-H2, and CDR-H3, of the VH sequences set forth in SEQ ID NO: 33, and a second variable light (VL) chain sequence comprising three light chain CDR sequences, CDR-L1, CDR-L2, and CDR-L3, of the VL sequence set forth in SEQ ID NO: 34, optionally wherein:
a. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

In one aspect, provided herein is a system comprising:
a. a first chimeric polypeptide comprises a priming receptor comprising a first antigen-binding domain that specifically binds Prostate Stem Cell Antigen (PSCA) (SEQ ID NO: 1), wherein the first antigen-binding domain comprises a variable heavy (VH) chain sequence comprising three heavy chain CDR sequences, CDR-H1, CDR-H2, and CDR-H3, of the VH sequences set forth in SEQ ID NO: 45, and a variable light (VL) chain sequence comprising three light chain CDR sequences, CDR-L1, CDR-L2, and CDR-L3, of the VL sequences set forth in SEQ ID NO: 46, optionally wherein:
   i. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52, and
b. a second chimeric polypeptide comprises a chimeric antigen receptor (CAR) comprising a second antigen-binding domain that specifically binds to Prostate-Specific Membrane Antigen (PSMA) (SEQ ID NO: 2), wherein the antigen-binding domain comprises a single domain antibody comprising a variable heavy (VH) chain sequence comprising three heavy chain CDR sequences, CDR-H1, CDR-H2, and CDR-H3 of the VH sequences set forth in SEQ ID NO: 33, and a variable light (VL) chain sequence comprising three light chain CDR sequences, CDR-L1, CDR-L2, and CDR-L3 of the VL sequence set forth in SEQ ID NO: 34, optionally wherein:
   i. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

In an embodiment, the first VH comprises a sequence at least 80%, 85%, 90%, 91%, 92%, 93%, 94%, 95%, 96%, 97%, 98%, 99%, or 100% identical to the sequence as set forth in SEQ ID NO: 45.

In an embodiment, the first VL comprises a sequence at least 80%, 85%, 90%, 91%, 92%, 93%, 94%, 95%, 96%, 97%, 98%, 99%, or 100% identical to the sequence as set forth in SEQ ID NO: 46.

In an embodiment, the first antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 44.

In an embodiment, the second VH comprises a sequence at least 80%, 85%, 90%, 91%, 92%, 93%, 94%, 95%, 96%, 97%, 98%, 99%, or 100% identical to the sequence as set forth in SEQ ID NO: 33.

In an embodiment, the second VL comprises a sequence at least 80%, 85%, 90%, 91%, 92%, 93%, 94%, 95%, 96%, 97%, 98%, 99%, or 100% identical to the sequence as set forth in SEQ ID NO: 34.

In an embodiment, the second antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 32.

In one aspect, provided herein is a system comprising:
a. a first chimeric polypeptide comprises a priming receptor comprising a first antigen-binding domain that specifically binds Prostate Stem Cell Antigen (PSCA) (SEQ ID NO: 1), wherein the first antigen-binding domain comprises a variable heavy (VH) chain sequence comprising three heavy chain CDR sequences, CDR-H1, CDR-H2, and CDR-H3, of the VH sequences set forth in SEQ ID NO: 45, and a variable light (VL) chain sequence comprising three light chain CDR sequences, CDR-L1, CDR-L2, and CDR-L3, of the VL sequences set forth in SEQ ID NO: 46, optionally wherein:
   i. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52.
b. a second chimeric polypeptide comprises a chimeric antigen receptor (CAR);
c. an optional third chimeric polypeptide comprising a synthetic pathway activator (SPA); and
d. at least one or more nucleic acids comprising a nucleic acid sequence at least 15 nucleotides in length complementary to:
   i. a nucleic acid encoding human Fas Cell Surface Death Receptor (FAS) comprising the sequence set forth in SEQ ID NO: 3; and
   ii. a nucleic acid encoding human Transforming Growth factor (TGF)-B Receptor 2 (TGFBR2) comprising the sequence set forth in SEQ ID NO: 4.

In an embodiment, the VH chain sequence comprises a sequence at least 80%, 85%, 90%, 91%, 92%, 93%, 94%, 95%, 96%, 97%, 98%, 99%, or 100% identical to the sequence as set forth in SEQ ID NO: 45.

In an embodiment, the VL chain sequence comprises a sequence at least 80%, 85%, 90%, 91%, 92%, 93%, 94%, 95%, 96%, 97%, 98%, 99%, or 100% identical to the sequence as set forth in SEQ ID NO: 46.

In an embodiment, the first antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 44.

In an embodiment, the CAR comprises a second antigen-binding domain that specifically binds to Prostate-Specific Membrane Antigen (PSMA) (SEQ ID NO: 2).

In an embodiment, the second antigen-binding domain comprises a variable heavy (VH) chain sequence comprising three heavy chain CDR sequences, CDR-H1, CDR-H2, and CDR-H3, of the VH sequences set forth in SEQ ID NO: 33, and a variable light (VL) chain sequence comprising three light chain CDR sequences, CDR-L1, CDR-L2, and CDR-L3, of the VL sequence set forth in SEQ ID NO: 34, optionally wherein:
a. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

In an embodiment, the VH comprises a sequence at least 80%, 85%, 90%, 91%, 92%, 93%, 94%, 95%, 96%, 97%, 98%, 99%, or 100% identical to the sequence as set forth in SEQ ID NO: 33.

In an embodiment, the VL comprises a sequence at least 80%, 85%, 90%, 91%, 92%, 93%, 94%, 95%, 96%, 97%, 98%, 99%, or 100% identical to the sequence as set forth in SEQ ID NO: 34.

In an embodiment, the second antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 32.

In one aspect, provided herein is a system comprising:
a. a first chimeric polypeptide comprises a priming receptor, and
b. a second chimeric polypeptide comprises a chimeric antigen receptor (CAR) comprising a second antigen-binding domain that specifically binds to Prostate-Specific Membrane Antigen (PSMA) (SEQ ID NO: 2), wherein the antigen-binding domain comprises a single domain antibody comprising a variable heavy (VH) chain sequence comprising three heavy chain CDR sequences, CDR-H1, CDR-H2, and CDR-H3 of the VH sequences set forth in SEQ ID NO: 33, and a variable light (VL) chain sequence comprising three light chain CDR sequences, CDR-L1, CDR-L2, and CDR-L3 of the VL sequence set forth in SEQ ID NO: 34, optionally wherein:
   i. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40;
c. an optional third chimeric polypeptide comprising a synthetic pathway activator (SPA); and
d. at least one or more nucleic acids comprising a nucleic acid sequence at least 15 nucleotides in length complementary to:
   i. a nucleic acid encoding human Fas Cell Surface Death Receptor (FAS) comprising the sequence set forth in SEQ ID NO: 3; and
   ii. a nucleic acid encoding human Transforming Growth factor (TGF)-B Receptor 2 (TGFBR2) comprising the sequence set forth in SEQ ID NO: 4.

In an embodiment, the nucleic acid sequence is complementary to nucleotides 1126 to 1364 of a nucleic acid encoding human FAS comprising the sequence set forth in SEQ ID NO: 3.

In an embodiment, the VH chain sequence comprises a sequence at least 80%, 85%, 90%, 91%, 92%, 93%, 94%, 95%, 96%, 97%, 98%, 99%, or 100% identical to the sequence as set forth in SEQ ID NO: 33.

In an embodiment, the VL comprises a sequence at least 80%, 85%, 90%, 91%, 92%, 93%, 94%, 95%, 96%, 97%, 98%, 99%, or 100% identical to the sequence as set forth in SEQ ID NO: 34.

In an embodiment, the second antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 32.

In an embodiment, the priming receptor comprises a first antigen-binding domain that specifically binds to Prostate Stem Cell Antigen (PSCA).

In an embodiment, the first antigen-binding domain comprises a variable heavy (VH) chain sequence comprising three heavy chain CDR sequences, CDR-H1, CDR-H2, and CDR-H3, of the VH sequences set forth in SEQ ID NO: 45, and a variable light (VL) chain sequence comprising three light chain CDR sequences, CDR-L1, CDR-L2, and CDR-L3, of the VL sequences set forth in SEQ ID NO: 46, optionally wherein:
a. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52.

In an embodiment, the VH comprises a sequence at least 80%, 85%, 90%, 91%, 92%, 93%, 94%, 95%, 96%, 97%, 98%, 99%, or 100% identical to the sequence as set forth in SEQ ID NO: 45.

In an embodiment, the VL comprises a sequence at least 80%, 85%, 90%, 91%, 92%, 93%, 94%, 95%, 96%, 97%, 98%, 99%, or 100% identical to the sequence as set forth in SEQ ID NO: 46.

In an embodiment, the first antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 44.

In an embodiment, the priming receptor comprises, from N-terminus to C-terminus,
a. the first antigen-binding domain;
b. a first transmembrane domain comprising one or more ligand-inducible proteolytic cleavage sites; and
c. an intracellular domain comprising a human or humanized transcriptional effector, wherein binding of PSCA by the first antigen-binding domain results in cleavage at the one or more ligand-inducible proteolytic cleavage sites.

In an embodiment, the priming receptor further comprises a first hinge domain positioned between the first antigen-binding domain and the first transmembrane domain.

In an embodiment, the first hinge domain comprises a CD8a or truncated CD8a hinge domain.

In an embodiment, the first hinge comprises the sequence as set forth in SEQ ID NO: 21.

In an embodiment, the first transmembrane domain comprises a Notch1 transmembrane domain.

In an embodiment, the transmembrane domain comprises the sequence as set forth in SEQ ID NO: 22.

In an embodiment, the intracellular domain comprises an HNF1a/p65 domain.

In an embodiment, the intracellular domain comprises the sequence as set forth in SEQ ID NO: 24, 25 or 26.

In an embodiment, the priming receptor further comprises a stop-transfer-sequence or juxtamembrane domain between the first transmembrane domain and the intracellular domain.

In an embodiment, the stop-transfer-sequence or juxtamembrane domain comprises the sequence as set forth in SEQ ID NO: 23.

In an embodiment, the priming receptor comprises a sequence as set forth in SEQ ID NO: 56.

In an embodiment, the CAR comprises, from N-terminus to C-terminus,
a. a second antigen-binding domain;
b. a second transmembrane domain;
c. an intracellular co-stimulatory domain; and
d. an intracellular activation domain.

In an embodiment, the CAR comprises a second hinge domain.

In an embodiment, the second hinge domain comprises a CD8a or truncated CD8a hinge domain.

In an embodiment, the second hinge domain comprises a sequence as set forth in SEQ ID NO: 9.

In an embodiment, the second transmembrane domain comprises a CD8a transmembrane domain.

In an embodiment, the second transmembrane domain comprises a sequence as set forth in SEQ ID NO: 10.

In an embodiment, the intracellular co-stimulatory domain comprises a 4-1BB domain.

In an embodiment, the intracellular co-stimulatory domain comprises a sequence as set forth in SEQ ID NO: 11.

In an embodiment, the intracellular activation domain comprises a CD3 °C domain.

In an embodiment, the intracellular activation domain comprises a sequence as set forth in SEQ ID NO: 12.

In an embodiment, the CAR comprises a sequence at least 80%, 85%, 90%, 91%, 92%, 93%, 94%, 95%, 96%, 97%, 98%, 99%, or 100% identical to the sequence as set forth in SEQ ID NO: 42.

In an embodiment, the at least one or more nucleic acid sequences are at least 16, 17, 18, 19, 20, 21, or 22 nucleotides in length.

In an embodiment, the at least one or more nucleic acid sequences are a short hairpin RNA (shRNA), a small interfering RNA (siRNA), a double stranded RNA (dsRNA), or an antisense oligonucleotide.

In an embodiment, the at least one or more nucleic acid sequences are shRNA.

In an embodiment, the nucleic acid sequence complementary to human FAS comprises a sequence as set forth in SEQ ID NO: 16.

In an embodiment, the nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In an embodiment, the nucleic acid sequence complementary to human TGFBR2 comprises one or more of the sequences as set forth in SEQ ID NOS: 17-19.

### Embodiment 58

In an embodiment, the nucleic acid sequence complementary to human TGFBR2 comprises each of the sequences as set forth in SEQ ID NOs: 17-19.

In an embodiment, the nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In an embodiment, the at least one or more nucleic acids comprises a sequence with at least 80%, 85%, 90%, 91%, 92%, 93%, 94%, 95%, 96%, 97%, 98%, 99%, or 100% identity to one or more of the sequences as set forth in SEQ ID NOs: 16-19.

In an embodiment, the system comprises at least two, at least three, or at least four of the nucleic acid sequences as set forth in SEQ ID NO: 16-19.

In an embodiment, the system comprises at least a first nucleic acid sequence complementary to a nucleic acid encoding human Fas Cell Surface Death Receptor (FAS) comprising the sequence set forth in SEQ ID NO: 3, and a second, third, and/or fourth nucleic acid sequence each of which are complementary to a nucleic acid encoding human TGF-β Receptor 2 (TGFBR2) comprising the sequence set forth in SEQ ID NO: 4.

In an embodiment, the first nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid and the second nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In an embodiment, the system comprises a sequence with at least 80%, 85%, 90%, 91%, 92%, 93%, 94%, 95%, 96%, 97%, 98%, 99%, or 100% identity to SEQ ID NO: 20.

In an embodiment, the SPA is an activator of STAT phosphorylation, optionally STAT1, STAT3, and/or STAT5 phosphorylation.

In an embodiment, the SPA comprises an antigen-binding domain linked to an intracellular signaling domain.

In an embodiment, the intracellular signaling domain comprises an intracellular signaling region derived from a cytokine receptor.

In an embodiment, the intracellular signaling domain comprises a polypeptide sequence derived from an interleukin receptor.

In an embodiment, the cytokine receptor comprises interleukin-6 signal transducer (IL6ST).

In an embodiment, the SPA comprises a sequence at least 80%, 85%, 90%, 91%, 92%, 93%, 94%, 95%, 96%, 97%, 98%, 99%, or 100% identical to the sequence as set forth in SEQ ID NO: 56.

In an embodiment, the antigen-binding domain conveys constitutive activity to the intracellular signaling domain.

In an embodiment, the system is encoded by a nucleic acid comprising a sequence with at least 80%, 85%, 90%, 91%, 92%, 93%, 94%, 95%, 96%, 97%, 98%, 99%, or 100% identity to a sequence as set forth in SEQ ID NO: 59 or 58.

In an embodiment, the nucleic acid comprises a sequence as set forth in SEQ ID NO: 58.

In an embodiment, the nucleic acid comprises a sequence as set forth in SEQ ID NO: 59.

In an embodiment, the priming receptor and the CAR are capable of binding to a same target cell if the target cell expresses PSCA and PSMA.

In an embodiment, the target cell is a human cell.

In an embodiment, the target cell is a cancer cell.

In an embodiment, the cancer cell is a solid tumor cell.

In an embodiment, the cancer is prostate cancer.

In an embodiment, the prostate cancer is metastatic.

In one aspect, provided herein is an antibody or antigen binding fragment thereof that binds to Prostate-Specific Membrane Antigen (PSMA) (SEQ ID NO: 2), comprising a variable heavy (VH) chain sequence comprising three heavy chain CDR sequences, CDR-H1, CDR-H2, and CDR-H3, of the VH sequences set forth in SEQ ID NO: 33, and a variable light (VL) chain sequence comprising three light chain CDR sequences, CDR-L1, CDR-L2, and CDR-L3, of the VL sequence set forth in SEQ ID NO: 34, optionally wherein:
a. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

In an embodiment, the VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 33, and the VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 34.

In an embodiment, the antibody or antigen binding fragment thereof comprises an scFv.

In an embodiment, the scFv comprises a sequence at least 80%, 85%, 90%, 91%, 92%, 93%, 94%, 95%, 96%, 97%, 98%, 99%, or 100% identical to the sequence as set forth in SEQ ID NO: 32.

In one aspect, provided herein is a receptor comprising the antibody or antigen binding fragment as defined in any of the embodiments described herein.

In an embodiment, the receptor is a chimeric antigen receptor (CAR) comprising, from N-terminus to C-terminus,
a. the extracellular antigen-binding domain;
b. a transmembrane domain;
c. an optional intracellular co-stimulatory domain; and
d. an intracellular activation domain.

In an embodiment, the CAR further comprises a hinge domain.

In an embodiment, the hinge domain comprises a CD8a, truncated CD8a, or CD28 hinge domain.

In an embodiment, the hinge domain comprises a sequence as set forth in SEQ ID NO: 9.

In an embodiment, the transmembrane domain comprises a CD8a transmembrane domain or a CD28 transmembrane domain.

In an embodiment, the transmembrane domain comprises a sequence as set forth in SEQ ID NO: 10.

In an embodiment, the intracellular co-stimulatory domain comprises a 4-1BB domain.

In an embodiment, the intracellular co-stimulatory domain comprises a sequence as set forth in SEQ ID NO: 11.

In an embodiment, the intracellular activation domain comprises a CD3 domain.

In an embodiment, the intracellular activation domain comprises a sequence as set forth in SEQ ID NO: 12.

In an embodiment, the CAR comprises a sequence at least 80%, 85%, 90%, 91%, 92%, 93%, 94%, 95%, 96%, 97%, 98%, 99%, or 100% identical to the sequence as set forth in SEQ ID NO: 42.

In one aspect, provided herein is an antibody or antigen binding fragment thereof that binds to Prostate Stem Cell Antigen (PSCA) (SEQ ID NO: 1), comprising a variable heavy (VH) chain sequence comprising three heavy chain CDR sequences, CDR-H1, CDR-H2, and CDR-H3, of the VH sequences set forth in SEQ ID NO: 45, and a variable light (VL) chain sequence comprising three light chain CDR sequences, CDR-L1, CDR-L2, and CDR-L3, of the VL sequences set forth in SEQ ID NO: 46, optionally wherein:
a. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52.

In an embodiment, the VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 45, and the VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 46.

In an embodiment, the antibody or antigen binding fragment thereof comprises an scFv.

In an embodiment, the scFv comprises a sequence at least 80%, 85%, 90%, 91%, 92%, 93%, 94%, 95%, 96%, 97%, 98%, 99%, or 100% identical to the sequence as set forth in SEQ ID NO: 44.

In one aspect, provided herein is a receptor comprising the antibody or antigen binding fragment as defined in any of the embodiments described herein.

In an embodiment, the receptor is a priming receptor comprising, from N-terminus to C-terminus,
a. the extracellular antigen-binding domain;
b. a transmembrane domain comprising one or more ligand-inducible proteolytic cleavage sites; and
c. an intracellular domain comprising a human or humanized transcriptional effector, wherein binding of PSCA by the extracellular antigen-binding domain results in cleavage at the one or more ligand-inducible proteolytic cleavage sites.

In an embodiment, the priming receptor further comprises a hinge domain positioned between the extracellular antigen-binding domain and the transmembrane domain.

In an embodiment, the hinge domain comprises a CD8a or truncated CD8a hinge domain.

In an embodiment, the hinge domain comprises the sequence as set forth in SEQ ID NO: 21.

In an embodiment, the transmembrane domain comprises a Notch1 transmembrane domain.

In an embodiment, the transmembrane domain comprises the sequence as set forth in SEQ ID NO: 22.

In an embodiment, the intracellular domain comprises an HNF1a/p65 domain.

In an embodiment, the intracellular domain comprises the sequence as set forth in SEQ ID NO: 24, 25 or 26.

In an embodiment, the priming receptor further comprises a stop-transfer-sequence or juxtamembrane domain between the transmembrane domain and the intracellular domain.

In an embodiment, the stop-transfer-sequence or juxtamembrane domain comprises the sequence as set forth in SEQ ID NO: 23.

In an embodiment, the priming receptor comprises a sequence at least 80%, 85%, 90%, 91%, 92%, 93%, 94%, 95%, 96%, 97%, 98%, 99%, or 100% identical to the sequence as set forth in SEQ ID NO: 54.

In one aspect, provided herein is a nucleic acid comprising a nucleotide sequence encoding the antibody as defined in any of the embodiments described herein.

In one aspect, provided herein is a nucleic acid comprising a nucleotide sequence encoding the antibody as defined in any of the embodiments described herein.

In one aspect, provided herein is a nucleic acid comprising a nucleotide sequence encoding the chimeric antigen receptor as defined in any of the embodiments described herein.

In one aspect, provided herein is a nucleic acid comprising a nucleotide sequence encoding the priming receptor as defined in any of the embodiments described herein.

In one aspect, provided herein is one or more nucleic acids comprising a nucleotide sequence encoding the system as defined in any of the embodiments described herein.

In an embodiment, the nucleic acid comprises a sequence with at least 80%, 85%, 90%, 91%, 92%, 93%, 94%, 95%, 96%, 97%, 98%, 99%, or 100% identity to a sequence selected from the group consisting of the sequences set forth in SEQ ID NO: 58.

In an embodiment, the nucleic acid comprises a sequence selected from the group consisting of the sequences set forth in SEQ ID NO: 58.

In one aspect, provided herein is one or more nucleic acids, wherein the one or more nucleic acids encode:
a. a first chimeric polypeptide comprising a priming receptor comprising a first extracellular antigen-binding domain that specifically binds Prostate Stem Cell Antigen (PSCA);
b. a second chimeric polypeptide comprising a chimeric antigen receptor (CAR) comprising a second extracellular antigen-binding domain that specifically binds to Prostate-Specific Membrane Antigen (PSMA);
c. an optional third chimeric polypeptide comprising a synthetic pathway activator (SPA); and
d. at least one nucleic acid sequence at least 15 nucleotides in length complementary to:
   i. a nucleic acid encoding human Fas Cell Surface Death Receptor (FAS) comprising the sequence set forth in SEQ ID NO: 3; and
   ii. a nucleic acid encoding human Transforming Growth factor (TGF)-B Receptor 2 (TGFBR2) comprising the sequence set forth in SEQ ID NO: 4.

In an embodiment, the first extracellular antigen-binding domain comprises a heavy chain comprising a first variable heavy (VH) chain sequence comprising three heavy chain CDR sequences, CDR-H1, CDR-H2, and CDR-H3, of the VH sequences set forth in SEQ ID NO: 45, and a light chain comprising a first variable light (VL) chain sequence comprising three light chain CDR sequences, CDR-L1, CDR-L2, and CDR-L3, of the VL sequences set forth in SEQ ID NO: 46, optionally wherein:
a. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52.

In an embodiment, the first VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 45, and the first VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 46.

In an embodiment, the first extracellular antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 44.

In an embodiment, the second extracellular antigen-binding domain comprises a heavy chain comprising a second variable heavy (VH) chain sequence comprising three heavy chain CDR sequences, CDR-H1, CDR-H2, and CDR-H3, of the VH sequences set forth in SEQ ID NO: 33, and a light chain comprising a second variable light (VL) chain sequence comprising three light chain CDR sequences, CDR-L1, CDR-L2, and CDR-L3, of the VL sequence set forth in SEQ ID NO: 34, optionally wherein:
a. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

In an embodiment, the second VH comprises a sequence at least 80%, 85%, 90%, 91%, 92%, 93%, 94%, 95%, 96%, 97%, 98%, 99%, or 100% identical to the sequence as set forth in SEQ ID NO: 33.

In an embodiment, the second VL comprises a sequence at least 80%, 85%, 90%, 91%, 92%, 93%, 94%, 95%, 96%, 97%, 98%, 99%, or 100% identical to the sequence as set forth in SEQ ID NO: 34.

In an embodiment, the second extracellular domain comprises a sequence at least 80%, 85%, 90%, 91%, 92%, 93%, 94%, 95%, 96%, 97%, 98%, 99%, or 100% identical to the sequence as set forth in SEQ ID NO: 32.

In an embodiment, the at least one nucleic acid sequences are at least 16, 17, 18, 19, 20, 21, or 22 nucleotides in length.

In an embodiment, the at least one nucleic acid sequences are a short hairpin RNA (shRNA), a small interfering RNA (siRNA), a double stranded RNA (dsRNA), or an antisense oligonucleotide.

In an embodiment, the at least one nucleic acid sequences are shRNA.

In an embodiment, the at least one or more nucleic acids comprises a sequence with at least 80%, 85%, 90%, 91%, 92%, 93%, 94%, 95%, 96%, 97%, 98%, 99%, or 100% identity to one or more of the sequences set forth in SEQ ID NOs: 16-19.

In an embodiment, the at least one or more nucleic acids comprises one or more of the sequences set forth in SEQ ID NOs: 16-19.

In an embodiment, the at least one or more nucleic acids comprises a sequence as set forth in SEQ ID NO: 16.

In an embodiment, the at least one or more nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In an embodiment, the at least one or more nucleic acid comprises one or more of the sequences set forth in SEQ ID NO: 17-19.

In an embodiment, the at least one or more nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In an embodiment, the at least one or more nucleic acid comprises a sequence with at least 80%, 85%, 90%, 91%, 92%, 93%, 94%, 95%, 96%, 97%, 98%, 99%, or 100% identity to SEQ ID NO: 20.

In an embodiment, the at least one or more nucleic acid sequence is encoded in at least one intron region of the nucleic acid.

In an embodiment, the nucleic acid comprises a sequence with at least 80%, 85%, 90%, 91%, 92%, 93%, 94%, 95%, 96%, 97%, 98%, 99%, or 100% identity to a sequence selected from the group consisting of the sequences set forth in SEQ ID NO: 58.

In an embodiment, the nucleic acid comprises a sequence selected from the group consisting of the sequences set forth in SEQ ID NO: 58.

In one aspect, provided herein is one or more nucleic acids comprising at least one nucleic acid fragment comprising a nucleotide sequence encoding a priming receptor comprising a first extracellular antigen-binding domain that specifically binds to PSCA, a nucleotide sequence encoding a chimeric antigen receptor comprising a second extracellular antigen-binding domain that specifically binds to PSMA, a synthetic pathway activator (SPA); and at least one nucleic acid sequence at least 15 nucleotides in length, wherein the at least one nucleic acid sequence comprises one or more of: (1) a first nucleic acid sequence complementary to a nucleic acid encoding human Fas Cell Surface Death Receptor (FAS) comprising the sequence set forth in SEQ ID NO: 3, and (2) a second, third, and/or fourth nucleic acid sequence each of which are complementary to a nucleic acid encoding human Transforming Growth factor (TGF)-ß Receptor 2 (TGFBR2) comprising the sequence set forth in SEQ ID NO: 4.

In one aspect, provided herein is one or more nucleic acids comprising at least one nucleic acid fragment comprising the nucleic acid(s) as defined in any of the embodiments described herein.

In an embodiment, the nucleic acid comprises two or more nucleic acid fragments.

In an embodiment, the nucleic acid further comprises an inducible promoter operably linked to the nucleotide sequence encoding the CAR.

In an embodiment, the nucleic acid further comprises a constitutive promoter operably linked to the nucleotide sequence encoding the priming receptor.

In an embodiment, the nucleic acid further comprises an inducible promoter operably linked to the nucleotide sequence encoding the chimeric antigen receptor and a constitutive promoter operably linked to the nucleotide sequence encoding the priming receptor.

In an embodiment, the constitutive promoter is an EF1a promoter.

In an embodiment, the EF1a promoter comprises a sequence as set forth in SEQ ID NO: 15.

In an embodiment, the inducible promoter comprises one or more Hepatocyte Nuclear Factor 1a (HNF1a) enhancer element(s).

In an embodiment, the inducible promoter further comprises a YB-TATA promoter sequence.

In an embodiment, the inducible promoter comprises a sequence as set forth in SEQ ID NO: 7.

In an embodiment, the nucleic acid comprises, in a 5′ to 3′ direction,
a. the constitutive promoter;
b. the nucleotide sequence encoding priming receptor;
c. the inducible promoter;
d. the nucleotide sequence encoding chimeric antigen receptor; and
e. the optional nucleotide sequence encoding the SPA.

In an embodiment, the nucleic acid comprises, in a 5′ to 3′ direction,
a. the inducible promoter;
b. the nucleotide sequence encoding chimeric antigen receptor;
c. the constitutive promoter; and
d. the nucleotide sequence encoding priming receptor; and
e. the optional nucleotide sequence encoding the SPA.

In an embodiment, the nucleic acid comprises, in a 5′ to 3′ direction,
a. the first constitutive promoter;
b. the nucleotide sequence encoding the priming receptor;
c. the second constitutive promoter;
d. the nucleotide sequence encoding the at least one nucleic acid complementary to human FAS or human TGFBR2;
e. the inducible promoter;
f. the optional nucleotide sequence encoding the chimeric antigen receptor; and
g. the nucleotide sequence encoding the SPA.

In an embodiment, the nucleic acid comprises, in a 5′ to 3′ direction,
a. the first constitutive promoter;
b. the nucleotide sequence encoding the priming receptor;
c. the second constitutive promoter;
d. the nucleotide sequence encoding the first nucleic acid complementary to human FAS or the nucleotide sequence encoding the second, third, and/or fourth nucleic acid each of which are complementary to human TGFBR2;
e. the nucleotide sequence encoding the first nucleic acid complementary to human FAS or the nucleotide sequence encoding the second, third, and/or fourth nucleic acid each of which are complementary to human TGFBR2;
f. the inducible promoter;
g. the nucleotide sequence encoding the chimeric antigen receptor; and
h. the optional nucleotide sequence encoding the SPA.

In an embodiment, the nucleic acid comprises, in a 5′ to 3′ direction,
a. the inducible promoter;
b. the nucleotide sequence encoding the chimeric antigen receptor;
c. the second constitutive promoter;
d. the nucleotide sequence encoding the first nucleic acid complementary to human FAS or the nucleotide sequence encoding the second, third, and/or fourth nucleic acid each of which are complementary to human TGFBR2;
e. the nucleotide sequence encoding the first nucleic acid complementary to human FAS or the nucleotide sequence encoding the second, third, and/or fourth nucleic acid each of which are complementary to human TGFBR2;
f. the first constitutive promoter; and
g. the nucleotide sequence encoding the priming receptor; and
h. the optional nucleotide sequence encoding the SPA.

In an embodiment, the nucleic acid further comprises a 5′ homology directed repair arm and a 3′ homology directed repair arm complementary to an insertion site in a host cell chromosome.

In an embodiment, the nucleic acid further comprises a woodchuck hepatitis virus post-translational regulatory element (WPRE).

In an embodiment, the WPRE is at the 3′ end of the nucleotide sequence encoding chimeric antigen receptor and at the 5′ end of the nucleotide sequence encoding priming receptor or wherein the WPRE is at the 3′ end of the nucleotide sequence encoding priming receptor and at the 5′ end of the nucleotide sequence encoding chimeric antigen receptor.

In an embodiment, the nucleic acid further comprises an SV40, a bovine growth hormone (BGH) or a human growth hormone (GH1) polyA element.

In an embodiment, the nucleic acid is incorporated into an expression cassette or an expression vector.

In an embodiment, the expression vector is a non-viral vector.

In one aspect, provided herein is a vector comprising the nucleic acid as defined in any of the embodiments described herein.

In an embodiment, the 5′ and 3′ ends of the nucleic acid comprise nucleotide sequences that are homologous to genomic sequences flanking an insertion site in a genome of a primary cell.

In an embodiment, the insertion site is located at a genomic safe harbor (GSH) locus or a T Cell Receptor Alpha Constant (TRAC) locus.

In an embodiment, the genomic safe harbor locus is a GS94 integration site, optionally wherein the GS94 integration site is at chr11:128340000-128350000.

In an embodiment, the 5′ and/or 3′ end of the nucleic acid comprises the sequence as set forth in SEQ ID NO: 5.

In one aspect, provided herein is a cell comprising:
a. the system as defined in any of the embodiments described herein;
b. at least one nucleic acid as defined in any of the embodiments described herein; and/or
c. the vector as defined in any of the embodiments described herein.

In an embodiment, the cell is an immune cell.

In one aspect, provided herein is an immune cell comprising:
a. the system as defined in any of the embodiments described herein;
b. at least one nucleic acid as defined in any of the embodiments described herein; and/or
c. the vector as defined in any of the embodiments described herein.

In an embodiment, the immune cell is a primary human immune cell.

In an embodiment, the immune cell is an allogeneic immune cell.

In an embodiment, the immune cell is an autologous immune cell.

In an embodiment, the primary immune cell is a natural killer (NK) cell, a T cell, a CD8+ T cell, a CD4+ T cell, a primary T cell, or a T cell progenitor.

In an embodiment, the primary immune cell is a primary T cell.

In an embodiment, the primary immune cell is a primary human T cell.

In an embodiment, the primary immune cell is virus-free.

In one aspect, provided herein is a primary immune cell comprising at least one nucleic acid comprising a priming receptor comprising a first extracellular antigen-binding domain that specifically binds to PSCA, a chimeric antigen receptor comprising a second extracellular antigen-binding domain that specifically binds to PSMA, and optionally a synthetic pathway activator inserted into a target region of the genome of the primary immune cell, and wherein the primary immune cell does not comprise a viral vector for introducing the nucleic acid into the primary immune cell.

In one aspect, provided herein is a viable, virus-free, primary cell comprising a ribonucleoprotein complex (RNP) and a nucleic acid, wherein the RNP comprises a nuclease domain and a guide RNA (gRNA), wherein the nucleic acid comprises a priming receptor comprising a first extracellular antigen-binding domain that specifically binds to PSCA and a chimeric antigen receptor comprising a second extracellular antigen-binding domain that specifically binds to PSMA, and optionally a synthetic pathway activator and wherein the 5′ and 3′ ends of the nucleic acid comprise nucleotide sequences that are homologous to genomic sequences flanking an insertion site in the genome of the primary cell.

In an embodiment, the primary cell further comprises at least one nucleic acid sequence at least 15 nucleotides in length, wherein the at least one nucleic acid sequence comprises one or more of a first nucleic acid sequence complementary to a nucleic acid encoding human Fas Cell Surface Death Receptor (FAS) comprising the sequence set forth in SEQ ID NO: 3, and a second nucleic acid sequence complementary to a nucleic acid encoding human Transforming Growth factor (TGF)-ß Receptor 2 (TGFBR2) comprising the sequence set forth in SEQ ID NO: 4.

In an embodiment, the nucleic acid comprises a sequence with at least 80%, 85%, 90%, 91%, 92%, 93%, 94%, 95%, 96%, 97%, 98%, 99%, or 100% identity to a sequence as set forth in SEQ ID NO: 59 or 58.

In an embodiment, the nucleic acid comprises a sequence with at least 80%, 85%, 90%, 91%, 92%, 93%, 94%, 95%, 96%, 97%, 98%, 99%, or 100% identity to a sequence as set forth in SEQ ID NO: 59.

In one aspect, provided herein is a population of cells comprising a plurality of cells or immune cells as defined in any of the embodiments described herein.

In one aspect, provided herein is a pharmaceutical composition comprising the cells or immune cell as defined in any of the embodiments described herein or the population of cells as defined in any of the embodiments described herein, and a pharmaceutically acceptable excipient.

In one aspect, provided herein is a pharmaceutical composition comprising the nucleic acid as defined in any of the embodiments described herein or the vector as defined in any of the embodiments described herein, and a pharmaceutically acceptable excipient.

In one aspect, provided herein is a method of editing a cell, comprising inserting the nucleic acid as defined in any of the embodiments described herein into the cell.

In an embodiment, the nucleic acid is introduced to the cell non-virally.

In an embodiment, the nucleic acid is inserted into an insertion site in the genome of the cell.

In one aspect, provided herein is a method of editing a cell, comprising:
a. providing the nucleic acid as defined in any of the embodiments described herein; and
b. editing the cell via insertion of the nucleic acid(s) into the cell.

In one aspect, provided herein is a method of editing a cell, comprising:
a. providing a nuclease domain and a guide RNA (gRNA), wherein the nucleic acid comprises the nucleic acid as defined in any of the embodiments described herein, and wherein the 5′ and 3′ ends of the nucleic acid comprise nucleotide sequences that are homologous to genomic sequences flanking an insertion site in the genome of the cell;
b. introducing the nuclease domain and nucleic acid into the cell, wherein the guide RNA specifically hybridizes to a target region of the genome of the cell, and wherein the nuclease domain cleaves the target region to create the insertion site in the genome of the cell; and
c. editing the cell via insertion of the nucleic acid into the insertion site in the genome of the cell.

In an embodiment, the nuclease domain and nucleic acid are introduced to the cell non-virally.

In one aspect, provided herein is a method of editing an immune cell, comprising:
a. providing a ribonucleoprotein complex (RNP) and a nucleic acid, wherein the RNP comprises a nuclease domain and a guide RNA (gRNA), wherein the nucleic acid comprises the nucleic acid as defined in any of the embodiments described herein, and wherein the 5′ and 3′ ends of the nucleic acid comprise nucleotide sequences that are homologous to genomic sequences flanking an insertion site in the genome of the immune cell;
b. non-virally introducing the RNP complex and nucleic acid into the immune cell, wherein the guide RNA specifically hybridizes to a target region of the genome of the primary immune cell, and wherein the nuclease domain cleaves the target region to create the insertion site in the genome of the immune cell; and
c. editing the immune cell via insertion of the nucleic acid as defined in any of the embodiments described herein into the insertion site in the genome of the immune cell.

In an embodiment non-virally introducing comprises electroporation.

In an embodiment, the nuclease domain comprises a CRISPR-associated endonuclease (Cas), optionally a Cas9 nuclease.

In an embodiment, the target region of the genome of the cell is a genomic safe harbor (GSH) locus or a T Cell Receptor Alpha Constant (TRAC) locus.

In an embodiment, the genomic safe harbor locus is a GS94 integration site, optionally wherein the GS94 integration site is at chr11:128340000-128350000.

In an embodiment, the gRNA comprises the sequence as set forth in SEQ ID NO: 5.

In an embodiment, the nucleic acid is a double-stranded nucleic acid or a single-stranded nucleic acid.

In an embodiment, the nucleic acid is a linear nucleic acid or a circular nucleic acid, optionally wherein the circular nucleic acid is a plasmid.

In an embodiment, the cell is an immune cell, optionally a primary human immune cell.

In an embodiment, the immune cell is an autologous immune cell.

In an embodiment, the immune cell is an allogeneic immune cell.

In an embodiment, the immune cell is a natural killer (NK) cell, a T cell, a CD8+ T cell, a CD4+ T cell, a primary T cell, or a T cell progenitor.

In an embodiment, the immune cell is a primary T cell.

In an embodiment, the immune cell is a primary human T cell.

In an embodiment, the cell is virus-free.

In an embodiment, the method further comprises obtaining the cell from a patient and introducing the nucleic acid in vitro.

In one aspect, provided herein is a method of inhibiting a target cell in a subject comprising administering the immune cell or population of immune cells as defined in any of the embodiments described herein to the subject, wherein the immune cell inhibits the target cell.

In an embodiment, the target cell expresses PSMA.

In an embodiment, the target cell is a cancer cell.

In one aspect, provided herein is a method of treating a disease in a subject comprising administering the cells or immune cell or population of cells or immune cells as defined in any of the embodiments described herein or the pharmaceutical composition as defined in any of the embodiments described herein to the subject.

In one aspect, provided herein is a method of treating a disease in a subject comprising:
a. determining or having determined the presence of PSCA-positive (PSCA+) cells from a cancer sample obtained from the subject; and/or
b. determining or having determined the presence of PSMA-positive (PSMA+) cells from a cancer sample obtained from the subject; and
c. administering the cell or immune cell as defined in any of the embodiments described herein or the pharmaceutical composition as defined in any of the embodiments described herein to the subject.

In an embodiment, the disease is cancer.

In an embodiment, the cancer is a solid tumor.

In an embodiment, the cancer is prostate cancer.

In an embodiment, the prostate cancer is metastatic.

In one aspect, provided herein is a method of inducing expression of a chimeric antigen receptor with a priming receptor in a cell comprising:
a. obtaining a cell or immune cell comprising
   i. the system as defined in any of the embodiments described herein;
   ii. the nucleic acid as defined in any of the embodiments described herein; and/or
   iii. the vector as defined in any of the embodiments described herein; and
b. contacting the cell or immune cell with a cell expressing PSCA, wherein binding of the priming receptor to PSCA on the cell induces activation of the priming receptor and expression of the chimeric antigen receptor.

In one aspect, provided herein is a method of modulating the activity of a cell or immune cell comprising:
a. obtaining a cell or immune cell comprising
   i. the system as defined in any of the embodiments described herein;
   ii. the nucleic acid as defined in any of the embodiments described herein; and/or
   iii. the vector as defined in any of the embodiments described herein; and
b. contacting the cell or immune cell with a cell expressing PSCA, wherein binding of the priming receptor to PSCA on the cell induces activation of the priming receptor and expression of the chimeric antigen receptor and wherein binding of the chimeric antigen receptor to PSMA on a target cell expressing PSMA modulates the activity of the immune cell.

In an embodiment, the administration of the immune cell enhances an immune response in the subject.

In an embodiment, the enhanced immune response is an adaptive immune response.

In an embodiment, the enhanced immune response is an innate immune response.

In an embodiment, the enhanced immune response is anti-tumor immune cell recruitment and activation.

In an embodiment, the method further comprises administering an immunotherapy to the subject concurrently with the immune cell or subsequently to the immune cell.

In an embodiment, the cell or immune cell proliferates in the subject after administration.

In an embodiment, the method further comprises determining the number of the cells or immune cells comprising the system as defined in any of the embodiments described herein, the nucleic acid as defined in any of the embodiments described herein, and/or the vector as defined in any of the embodiments described herein in the subject after administration, optionally wherein the number of the cells or immune cells is determined at least or about 5, 6, 7, 8, 9, or 10 days after administration.

### BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 shows an exemplary cassette encoding a logic gate comprising a priming receptor (primeR) and a chimeric antigen receptor (CAR), a quad Fas/3×TGFBR2 shRNA module and a synthetic pathway activator (SPA).

FIG. 2A shows flow plots of all ICT cells induce PSMA CAR expression when co-cultured with PSCA protein. Logic gate 1 (LG1) and Logic gate 5 (LG5) maintain high levels of PrimeR expression upon CAR induction. FIG. 2B shows flow plots of Logic gate 1 (LG 1) ICT cells reduced PSMA CAR expression over time upon removal from PSCA protein.

FIG. 3 shows cytotoxicity of indicated constructs against heterogeneous co-cultures of PC3 cells expressing tumor-matched levels of PSCA and PSMA mixed with non-PSCA expressing PC3 cells at the indicated percentage.

FIG. 4 shows cytotoxicity of indicated constructs against 786-O cells expressing PSCA at the indicated effector to target (E:T) ratios.

FIG. 5A shows cytotoxicity of LG 1 ICT cells against cells with medium or high PSCA and medium or high PSMA expression levels. FIG. 5B shows the expression levels of PSMA or PSCA in the cell lines used for the cytotoxicity analysis in FIG. 5A.

FIG. 6A shows GM-CSF, IFNγ and TNFα production from LG 1 ICT cells with and without a SPA after incubation with target PC3 tumor cells at a 1:1 ratio. FIG. 6B shows per cell cytokine levels after incubation of target PC3 tumor cells with LG 1 T cells with and without a SPA at a 1:1 ratio. FIG. 6C shows total cytokine levels after incubation of target PC3 tumor cells with LG 1 T cells with and without a SPA at a 1:1 ratio. In both, cytokine levels in LG 1 T cells with no target cell incubation is shown as a negative control.

FIG. 7A shows the tumor growth inhibition of 786-O-PSMA+ (left panel) and 786-O-PSCA+/PSMA+ (right panel) cells in a dual flank mouse model after treatment with 1×10⁶ ICTs expressing Logic Gates 1-5, PSMA CAR+ DNR, RNP or PBS generated from donor 1. FIG. 7B shows the tumor growth inhibition of 786-O-PSMA+ (left panel) and 786-O-PSCA+/PSMA+ (right panel) cells in a dual flank mouse model after treatment with ICTs expressing Logic Gates 1-5, PSMA CAR+ DNR, RNP or PBS generated from donor 2.

FIG. 8A shows the individual mouse tumor growth inhibition of 786-O-PSMA+ cells in a PSMA single flank mouse model after treatment with 1×10⁶ ICTs expressing LG 1-5, PSMA CAR+DNR, or RNP from donor 1. FIG. 8B shows the individual mouse tumor growth inhibition of 786-O-PSMA+ cells in a single flank mouse model after treatment with 0.3×10⁶ ICTs expressing LG 1-5, PSMA CAR+DNR, or RNP from donor 1. LG 2, LG 4 and LG 5 T cells showed killing of cells expressing the PSMA cytolytic antigen in the absence of PSCA priming, which was not observed in the LG 1 or LG 3 T cells.

FIG. 9A shows the individual mouse tumor growth of 786-O-PSCA+/PSMA-cells in a PSCA single flank mouse model after treatment with ICTs expressing LG 1-5, PSCA CAR, or RNP from donor 1. FIG. 9B shows the individual mouse tumor growth of 786-O-PSCA+/PSMA− cells in a single flank mouse model after treatment with ICTs expressing LG 1-5, PSCA CAR, or RNP from donor 2. None of the LG 1-5 T cells showed killing of cells expressing the PSCA priming antigen only.

FIG. 10A shows tumor growth inhibition of PC3-PSCA+/PSMA+ cells in a single flank mouse model after treatment with ICTs expressing LG 1-5, PSMA CAR+DNR, RNP or PBS from donor 1. FIG. 10B shows tumor growth inhibition of PC3-PSCA+/PSMA+ cells in a single flank mouse model after treatment with ICTs expressing LG 1-5, PSMA CAR+DNR, RNP or PBS from donor 2.

FIG. 11A shows the individual mouse tumor growth inhibition of PC3-PSCA+/PSMA+ cells in a single flank mouse model after treatment with 0.75×10⁵ ICTs expressing LG 1-5, PSMA CAR+DNR, or RNP from donor 1. FIG. 11B shows the individual mouse tumor growth inhibition of PC3-PSCA+/PSMA+ cells in a single flank mouse model after treatment with 0.75×10⁵ ICTs expressing LG 1-5, PSMA CAR+DNR, or RNP from donor 2.

FIG. 12A shows tumor growth inhibition of PC3-PSCA+/PSMA+ cells in a single flank mouse model after treatment with 0.75×10⁵ or 0.3×10⁶ ICTs expressing LG 1, PSMA CAR+DNR, RNP or PBS from donor 1. FIG. 12B shows tumor growth inhibition of PC3-PSCA+/PSMA+ cells in a single flank mouse model after treatment with 0.75×10⁵ or 0.3×10⁶ ICTs expressing LG 1, PSMA CAR+DNR, RNP or PBS from donor 2.

FIG. 13A shows the Kaplan Meier probability of survival curves for mice in the PC3-PSCA+/PSMA+ tumor model treated with LG 1 ICT cells, PSMA CAR+DNR, RNP or PBS from donor 3. FIG. 13B shows the Kaplan Meier probability of survival curves for mice in the PC3-PSCA+/PSMA+ tumor model treated with LG 1 ICT cells, PSMA CAR+DNR, RNP or PBS from donor 4. Mice treated with the LG 1 T cells showed the highest probability of survival.

FIG. 14 shows tumor growth inhibition of PC3-PSCA+/PSMA+ cells in an intratibial tumor bone mouse model after treatment with ICT cells expressing LG 1, RNP or PBS.

FIG. 15 shows ex-vivo flow cytometry quantification of PrimeR+ T cells per tibia in non-tumor and tumor tibia for mice after treatment with ICTs expressing LG 1 or RNP.

### DETAILED DESCRIPTION

### Definitions

Terms used in the claims and specification are defined as set forth below unless otherwise specified.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS). As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in International Patent Application WO2022093846A1, published on May 5, 2022, hereby incorporated by reference in its entirety.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chloroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 nuclease domain can be optimized for efficient activity or enhanced stability in the host cell.

As used herein, the term “Cas9” refers to an RNA-mediated nuclease (e.g., of bacterial or archeal origin, or derived therefrom). Exemplary RNA-mediated nucleases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+cell. T cells can be CD4⁺, CD8⁺, or CD4⁺ and CD8⁺. The T cell can be any type of T cell, CD4+/CD8+double positive T cells, CD4+helper T cells (e.g. Th1 and Th2 cells), CD8+T cells (e.g. cytotoxic T cells), peripheral Including but not limited to blood mononuclear cells (PBMC), peripheral blood leukocytes (PBL), tumor infiltrating lymphocytes (TIL), memory T cells, naïve T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+T cells” refers to a subset of T cells that express CD4 on their surface and are associated with a cellular immune response. CD4+T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy 1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, a hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. As used, transgene can refer to a polynucleotide that encodes a polypeptide.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is an RNA molecule. When present as an RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, Hepatocyte Nuclear Factor 1α (HNF1α), YB-TATA, etc.). The promoter can be a constitutive promoter (e.g., CMV promoter, UBC promoter, EF1a promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a “DNA template,” “DNA template insert,” “single-stranded DNA template,” a “single-stranded DNA template insert,” a “double-stranded DNA template,” or a “double-stranded DNA template insert” refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site. In some embodiments, the DNA template or DNA template insert comprises a cassette or expression cassette comprising one or more modules that encode for transgenes and/or RNAi molecules disclosed herein.

The terms “expression vector,” “vector,” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette or cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” or “cassette” is a polynucleotide construct, generated recombinantly or chemically synthesized, comprising regulatory sequences operably linked to a selected polynucleotide to facilitate expression of the selected polynucleotide in a host cell. Such cassettes may include one or more modules comprising the selected polynucleotide, such as the transgenes (e.g., a CAR, priming receptor, and/or synthetic pathway activator described herein) and/or RNAi molecules (e.g., an shRNA) disclosed herein. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector. In some embodiments, the cassette is part of a DNA template insert.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “in situ” refers to processes that occur in a living cell growing separate from a living organism, e.g., growing in tissue culture.

The term “in vivo” refers to processes that occur in a living organism.

As used herein, the term “ex vivo” generally includes experiments or measurements made in or on living tissue, preferably in an artificial environment outside the organism, preferably with minimal differences from natural conditions.

The term “mammal” as used herein includes both humans and non-humans and include but is not limited to humans, non-human primates, canines, felines, murines, bovines, equines, and porcines.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., compositions described herein, cells described herein) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). For example, an antibody that “selectively binds” or “specifically binds” an antigen is an antigen-binding moiety that binds the antigen with high affinity and does not significantly bind other unrelated antigens. Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including, but not limited to, surface plasmon resonance (SPR) technology (e.g., BIACORE®) or biolayer interferometry (e.g., FORTEBIO®).

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The term “CDR” denotes a complementarity determining region as defined by at least one manner of identification to one of skill in the art.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); Martin (Enhanced Chothia or AbM) Abhinandan and Martin, Mol Immunol. 2008 August; 45(14):3832-9; MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegger and Plückthun, J. Mol. Biol., 2001, 309:657-70 (“AHo” numbering scheme); each of which is incorporated by reference in its entirety.

Table A provides the positions of CDR-L1, CDR-L2, CDR-L3, CDR-H1, CDR-H2, and CDR-H3 as identified by the Kabat, Chothia, AbM, Contact, and IMGT schemes. For CDR-H1, residue numbering is provided using both the Kabat and Chothia numbering schemes.

CDRs may be assigned, for example, using antibody numbering software, such as Abnum, available at bioinf.org.uk/abs/abnum/ and described in Abhinandan and Martin, Immunology, 2008, 45:3832-3839, incorporated by reference in its entirety, and AbYsis, available at abysis.org/abysis/sequence_input/key_annotation/key_annotation.cgi.

TABLE A
Residues in CDRs according to Kabat, Chothia,
AbM, Contact, and IMGT numbering schemes.

| CDR | Kabat | Chothia | AbM | Contact | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 (Kabat | H31-H35B | H26-H32 or | H26-H35B | H30-H35B | H26-H35B |
| Numbering) | | H34* | | | |
| H1 | H31-H35 | H26-H32 | H26-H35 | H30-H35 | H26-H33 |
| (Chothia/Martin | | | | | |
| Numbering) | | | | | |
| H2 | H50-H65 | H52-H56 | H50-H58 | H47-H58 | H51-H56 |
| H3 | H95-H102 | H95-H102 | H95-H102 | H93-H101 | H93-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

As used herein, the term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)₂” fragments contain two Fab′ fragments joined, near the hinge region, by disulfide bonds. F(ab′)₂ fragments may be generated, for example, by recombinant or synthetic methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

The “Single-chain Fv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22). The terms “single domain antibody” and “sdAb” are used interchangeably herein to refer to an antibody comprising at least one monomeric domain, such as a VHH domain, or a VNAR domain (from a shark antibody) without a light chain, and an Fc region.

The term “VHH” or “VHH domain” or “VHH antigen-binding domain” as used herein refers to the antigen-binding portion of a single-domain antibody (sdAb), such as a camelid antibody. In some embodiments, a VHH comprises three CDRs and four framework regions, designated FR1, CDR1, FR2, CDR2, FR3, CDR3, and FR4.

It must be noted that, as used in the specification and the appended claims, the singular forms “a,” “an” and “the” include plural referents unless the context clearly dictates otherwise.

### Antibodies and Antigen Binding Fragments

### PSCA Antibodies, Antigen Binding Fragments, CDRs, VH, and VL Domains

In some aspects, provided herein are antibodies or antigen binding fragments thereof that bind to Prostate Stem Cell Antigen (PSCA) (SEQ ID NO: 1). PSCA is also known as LNCPSCA or PRO232 (HGNC: 9500, NCBI Entrez Gene: 8000, Ensembl: ENSG00000167653, UniProtKB/Swiss-Prot: 043653). An exemplary amino acid sequence of human PSCA is provided in SEQ ID NO: 1. In some aspects, the antibodies or antigen binding fragments thereof that bind to Prostate Stem Cell Antigen are isolated.

In some aspects, provided herein are antibodies or antigen binding fragments thereof that bind to PSCA. In some aspects, provided herein are means for binding to PSCA. In some embodiments, the means for binding to PSCA comprises an antibody or antigen-binding fragment provided herein. In some embodiments, a PSCA antibody or antigen-binding fragment or equivalent thereof comprises means for binding a PSCA protein, optionally binding a human PSCA protein in the region(s) of human PSCA bound by the PSCA binders (e.g., as described in the Examples below). In some embodiments, the means binds a PSCA protein. In some embodiments, the means binds a human PSCA protein. In some embodiments, the means is a PSCA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding a PSCA protein. In some embodiments, the means for binding PSCA includes the anti-PSCA antibodies and antigen-binding fragments or equivalents thereof described herein.

In some embodiments, a PSCA antibody or antigen-binding fragment or equivalent thereof provided herein binds to human PSCA. In some embodiments, a PSCA antibody or antigen-binding fragment or equivalent thereof provided herein does not bind to mouse PSCA.

In some aspects, the PSCA antibody or antigen-binding fragment or equivalent thereof comprises a variable heavy (VH) chain sequence comprising three heavy chain CDR sequences, CDR-H1, CDR-H2, and CDR-H3, of the VH sequences set forth in SEQ ID NO: 45, and a variable light (VL) chain sequence comprising three light chain CDR sequences, CDR-L1, CDR-L2, and CDR-L3, of the VL sequences set forth in SEQ ID NO: 46. In some embodiments, CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52.

In some embodiments, the VH chain sequence comprises the sequence set forth in SEQ ID NO: 45. In some embodiments, the VL comprises the sequence set forth in SEQ ID NO: 46. In some embodiments, the PSCA antibody or antigen-binding fragment or equivalent thereof comprises the sequence set forth in SEQ ID NO: 44.

In some embodiments, the PSCA antibody or antigen-binding fragment or equivalent thereof CDR-H3 has at least about 50%, 75%, 80%, 85%, 90%, or 95% identity with a CDR-H3 of SEQ ID NO: 49, the CDR-H2 has at least about 50%, 75%, 80%, 85%, 90%, or 95% identity with a CDR-H2 of SEQ ID NO: 48, the CDR-H1 has at least about 50%, 75%, 80%, 85%, 90%, or 95% identity with a CDR-H1 of SEQ ID NO: 47, the CDR-L3 has at least about 50%, 75%, 80%, 85%, 90%, or 95% identity with a CDR-L3 of SEQ ID NO: 52, the CDR-L2 has at least about 50%, 75%, 80%, 85%, 90%, or 95% identity with a CDR-L2 of SEQ ID NO: 51, and the CDR-L1 has at least about 50%, 75%, 80%, 85%, 90%, or 95% identity with a CDR-L1 of SEQ ID NO: 50. In some embodiments, the CDR-H3 is a CDR-H3 of SEQ ID NO: 49, with up to 1, 2, 3, 4, 5, 6, 7, or 8 amino acid substitutions; the CDR-H2 is a CDR-H2 of SEQ ID NO: 48, with up to 1, 2, 3, 4, 5, 6, 7, or 8 amino acid substitutions; the CDR-H1 is a CDR-H1 of SEQ ID NO: 47, with up to 1, 2, 3, 4, or 5 amino acid substitutions; the CDR-L3 is a CDR-L3 of SEQ ID NO: 52, with up to 1, 2, 3, 4, or 5 amino acid substitutions; the CDR-L2 is a CDR-L2 of SEQ ID NO: 51, with up to 1, 2, 3, or 4 amino acid substitutions; and the CDR-L1 is a CDR-L1 of SEQ ID NO: 50 with up to 1, 2, 3, 4, 5, or 6 amino acid substitutions.

In some embodiments, a PSCA antibody or antigen-binding fragment or equivalent thereof provided herein comprises one to three CDRs of a VH domain as set forth in SEQ ID NO: 45. In some embodiments, a PSCA antibody or antigen-binding fragment or equivalent thereof provided herein comprises two to three CDRs of a VH domain as set forth in SEQ ID No: 45. In some embodiments, a PSCA antibody or antigen-binding fragment or equivalent thereof provided herein comprises three CDRs of a VH domain as set forth in SEQ ID NO: 45. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a PSCA antibody or antigen-binding fragment or equivalent thereof provided herein comprises a VH sequence having at least about 50%, 60%, 70%, 80%, 90%, 95%, or 99% identity to an VH sequence set forth in SEQ ID NO: 45. In some embodiments, an antigen-binding domain provided herein comprises a VH sequence provided in SEQ ID NO: 45, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSCA antibody or antigen-binding fragment or equivalent thereof provided herein comprises one to three CDRs of a VL domain as set forth in SEQ ID NO: 46. In some embodiments, an antigen-binding domain provided herein comprises two to three CDRs of a VL domain as set forth in SEQ ID NO: 46. In some embodiments, an antigen-binding domain provided herein comprises three CDRs of a VL domain as set forth in SEQ ID NO: 46. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a PSCA antibody or antigen-binding fragment or equivalent thereof provided herein comprises a VL sequence having at least about 50%, 60%, 70%, 80%, 90%, 95%, or 99% identity to an VL sequence set forth in SEQ ID NO: 46. In some embodiments, a PSCA antibody or antigen-binding fragment or equivalent thereof provided herein comprises a VL sequence provided in SEQ ID NO: 46, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSCA antibody or antigen-binding fragment or equivalent thereof provided herein comprises a sequence having at least about 50%, 60%, 70%, 80%, 90%, 95%, 99%, or 100% identity to the sequence set forth in SEQ ID NO: 44. In some embodiments, a PSCA antibody or antigen-binding fragment or equivalent thereof antigen-binding domain provided herein comprises an scFv sequence provided in SEQ ID NO: 44, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains. In some embodiments, a PSCA antibody or antigen-binding fragment or equivalent thereof provided herein comprises a nucleic acid sequence having at least about 50%, 60%, 70%, 80%, 90%, 95%, 99%, or 100% identity to the sequence set forth in SEQ ID NO: 53. In some embodiments, a PSCA antibody or antigen-binding fragment or equivalent thereof provided herein is encoded by the sequence set forth in SEQ ID NO: 53.

In some embodiments, a PSCA antibody or antigen-binding fragment or equivalent thereof comprises means for binding a PSCA protein, optionally binding a human PSCA protein in the region(s) of human PSCA bound by the PSCA antibody (e.g., as described in the Examples below). In some embodiments, the means binds human PSCA protein. In some embodiments, the means is a PSCA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding human PSCA protein. In some embodiments, the means for binding PSCA includes the anti-PSCA antibodies and antigen-binding fragments or equivalents thereof described herein.

### PSMA Antibodies, Antigen Binding Fragments, CDRs, VH, and VL Domains

In some aspects, provided herein are antibodies or antigen binding fragments thereof that bind to Prostate-Specific Membrane Antigen (PSMA) (SEQ ID NO: 2). PSMA is also known as FOLH1 or Folate Hydrolase 1 (HGNC: 3788, NCBI Entrez Gene: 2346, Ensembl: ENSG00000086205, UniProtKB/Swiss-Prot: Q04609). An exemplary amino acid sequence of human PSMA is provided in SEQ ID NO: 2. In some aspects, the antibodies or antigen binding fragments thereof that bind to Prostate-Specific Membrane Antigen are isolated.

In some aspects, provided herein are antibodies or antigen binding fragments thereof that bind to PSMA. In some aspects, provided herein are means for binding to PSMA. In some embodiments, the means for binding to PSMA comprises an antibody or antigen-binding fragment provided herein. In some embodiments, a PSMA antibody or antigen-binding fragment or equivalent thereof comprises means for binding a PSMA protein, optionally binding a human PSMA protein in the region(s) of human PSMA bound by the PSMA binders (e.g., as described in the Examples below). In some embodiments, the means binds a PSMA protein. In some embodiments, the means binds a human PSMA protein. In some embodiments, the means is a PSMA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding a PSMA protein. In some embodiments, the means for binding PSMA includes the anti-PSMA antibodies and antigen-binding fragments or equivalents thereof described herein.

In some embodiments, a PSMA antibody or antigen-binding fragment or equivalent thereof provided herein binds to human PSMA. In some embodiments, a PSMA antibody or antigen-binding fragment or equivalent thereof provided herein does not bind to mouse PSMA. In some embodiments, a PSMA antibody or antigen-binding fragment or equivalent thereof provided herein binds to isoforms of human PSMA, including, but not limited to, a PSMA protein comprising a Y75H single nucleotide polymorphism (SNP) as compared to SEQ ID NO: 2.

In some aspects, the PSMA antibody or antigen-binding fragment or equivalent thereof comprises a variable heavy (VH) chain sequence comprising three heavy chain CDR sequences, CDR-H1, CDR-H2, and CDR-H3, of the VH sequences set forth in SEQ ID NO: 33. In some embodiments, the PSMA antibody or antigen-binding fragment or equivalent thereof comprises a variable light (VL) chain sequence comprising three light chain CDR sequences, CDR-L1, CDR-L2, and CDR-L3, of the VL sequence set forth in SEQ ID NO: 34. In some embodiments, CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

In some embodiments, the VH chain sequence comprises the sequence set forth in SEQ ID NO: 33. In some embodiments, the VL comprises the sequence set forth in SEQ ID NO: 34. In some embodiments, the PSMA antibody or antigen-binding fragment or equivalent thereof comprises the sequence set forth in SEQ ID NO: 32.

In some embodiments, the PSMA antibody or antigen-binding fragment or equivalent thereof CDR-H3 has at least about 50%, 75%, 80%, 85%, 90%, or 95% identity with a CDR-H3 of SEQ ID NO: 37, the CDR-H2 has at least about 50%, 75%, 80%, 85%, 90%, or 95% identity with a CDR-H2 of SEQ ID NO: 36, the CDR-H1 has at least about 50%, 75%, 80%, 85%, 90%, or 95% identity with a CDR-H1 of SEQ ID NO: 35, the CDR-L3 has at least about 50%, 75%, 80%, 85%, 90%, or 95% identity with a CDR-L3 of SEQ ID NO: 40, the CDR-L2 has at least about 50%, 75%, 80%, 85%, 90%, or 95% identity with a CDR-L2 of SEQ ID NO: 39, and the CDR-L1 has at least about 50%, 75%, 80%, 85%, 90%, or 95% identity with a CDR-L1 of SEQ ID NO: 38. In some embodiments, the CDR-H3 is a CDR-H3 of SEQ ID NO: 37, with up to 1, 2, 3, 4, 5, 6, 7, or 8 amino acid substitutions; the CDR-H2 is a CDR-H2 of SEQ ID NO: 36, with up to 1, 2, 3, 4, 5, 6, 7, or 8 amino acid substitutions; the CDR-H1 is a CDR-H1 of SEQ ID NO: 35, with up to 1, 2, 3, 4, or 5 amino acid substitutions; the CDR-L3 is a CDR-L3 of SEQ ID NO: 40, with up to 1, 2, 3, 4, or 5 amino acid substitutions; the CDR-L2 is a CDR-L2 of SEQ ID NO: 39, with up to 1, 2, 3, or 4 amino acid substitutions; and the CDR-L1 is a CDR-L1 of SEQ ID NO: 38 with up to 1, 2, 3, 4, 5, or 6 amino acid substitutions.

In some embodiments, a PSMA antibody or antigen-binding fragment or equivalent thereof provided herein comprises one to three CDRs of a VH domain as set forth in SEQ ID NO: 33. In some embodiments, a PSMA antibody or antigen-binding fragment or equivalent thereof provided herein comprises two to three CDRs of a VH domain as set forth in SEQ ID NO: 33. In some embodiments, a PSMA antibody or antigen-binding fragment or equivalent thereof provided herein comprises three CDRs of a VH domain as set forth in SEQ ID NO: 33. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a PSMA antibody or antigen-binding fragment or equivalent thereof provided herein comprises a VH sequence having at least about 50%, 60%, 70%, 80%, 90%, 95%, or 99% identity to an VH sequence set forth in SEQ ID NO: 33. In some embodiments, an antigen-binding domain provided herein comprises a VH sequence provided in SEQ ID NO: 33, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSMA antibody or antigen-binding fragment or equivalent thereof provided herein comprises one to three CDRs of a VL domain as set forth in SEQ ID NO: 34. In some embodiments, an antigen-binding domain provided herein comprises two to three CDRs of a VL domain as set forth in SEQ ID NO: 34. In some embodiments, an antigen-binding domain provided herein comprises three CDRs of a VL domain as set forth in SEQ ID NO: 34. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a PSMA antibody or antigen-binding fragment or equivalent thereof provided herein comprises a VL sequence having at least about 50%, 60%, 70%, 80%, 90%, 95%, or 99% identity to an VL sequence set forth in SEQ ID NO: 34. In some embodiments, a PSMA antibody or antigen-binding fragment or equivalent thereof provided herein comprises a VL sequence provided in SEQ ID NO: 34, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSMA antibody or antigen-binding fragment or equivalent thereof provided herein comprises a sequence having at least about 50%, 60%, 70%, 80%, 90%, 95%, 99%, or 100% identity to the sequence set forth in SEQ ID NO: 32. In some embodiments, a PSMA antibody or antigen-binding fragment or equivalent thereof provided herein comprises a nucleic acid sequence having at least about 50%, 60%, 70%, 80%, 90%, 95%, 99%, or 100% identity to the sequence set forth in SEQ ID NO: 41. In some embodiments, a PSMA antibody or antigen-binding fragment or equivalent thereof provided herein is encoded by the sequence set forth in SEQ ID NO: 41. In some embodiments, a PSMA antibody or antigen-binding fragment or equivalent thereof antigen-binding domain provided herein comprises an scFv sequence provided in SEQ ID NO: 32, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a PSMA antibody or antigen-binding fragment or equivalent thereof comprises means for binding a PSMA protein, optionally binding human PSMA protein in the region(s) of human PSMA protein bound by the PSMA antibody (e.g., as described in the Examples below). In some embodiments, the means binds human PSMA. In some embodiments, the means is a PSMA antibody or antigen-binding fragment or equivalent thereof (e.g., a full length antibody or a F(ab′)2 fragment, a Fab fragment, a single chain variable fragment (scFv), a VHH, and a single domain antibody (sdAb), or a functional fragment thereof) means for binding PSMA. In some embodiments, the means for binding PSMA includes the anti-PSMA antibodies and antigen-binding fragments or equivalents thereof described herein.

### Logic Gate Systems

As used herein, a “logic gate,” “circuit,” “circuit receptor,” “system” or “system receptor” refers to a two part protein expression system comprising a priming receptor and a chimeric antigen receptor. The system can be encoded on at least one nucleic acid inserted into a cell, where the priming receptor is expressed in the cell. The intracellular domain of the priming receptor is cleaved from the transmembrane domain upon binding of the priming receptor to its target antigen. The intracellular domain is then capable of translocating into a cell nucleus where it induces expression of the chimeric antigen receptor.

In one aspect, provided herein are systems comprising a priming receptor that binds to PSCA and a chimeric antigen receptor that binds to PSMA, wherein the transcription factor of the intracellular domain of the priming receptor is capable of inducing expression of the CAR. Such systems are alternatively termed “logic gates” or “circuits.” In some aspects, the system is encoded by nucleic acid transgenes inserted into an immune cell. The system can be encoded on a single nucleic acid insert or fragment that comprises both transgenes, or can be encoded on two nucleic acids that encode the system transgenes individually. The priming receptor and CAR of the system can be placed in any order on the single nucleic acid. For example, the priming receptor can be at the 5′ end and the CAR can be at the 3′ end, or the CAR can be at the 5′ end and the priming receptor can be at the 3′ end.

A constitutive promoter can be operably linked to the nucleotide sequence encoding the priming receptor. An inducible promoter can also be operably linked to the nucleotide sequence encoding the CAR. In some embodiments, when the system is encoded on a single nucleic acid insert or fragment that comprises both transgenes, the nucleic acid can comprise, in a 5′ to 3′ direction, the constitutive promoter; the nucleotide sequence encoding priming receptor; the inducible promoter; and the nucleotide sequence encoding chimeric antigen receptor. Alternatively, the nucleic acid can comprise, in a 5′ to 3′ direction, the inducible promoter; the nucleotide sequence encoding chimeric antigen receptor; the constitutive promoter; the nucleotide sequence encoding priming receptor.

In some embodiments, the constitutive promoter is an EF1a promoter. In some embodiments, the constitutive promoter comprises the sequence of SEQ ID NO: 15.

In some embodiments, the inducible promoter comprises one or more Hepatocyte Nuclear Factor 1α (HNF1α) enhancer element(s). For example, the inducible promoter can comprise 1, 2, 3, 4, 5, 6, 7, or more HNF1α enhancer element(s). In some embodiments, the inducible promoter further comprises a YB-TATA promoter sequence. In some embodiments, the inducible promoter comprises the sequence as set forth in SEQ ID NO: 7.

In some embodiments, the logic gate is encoded on a nucleic acid. In some embodiments, the nucleic acid comprises a sequence as set forth in SEQ ID NO: 58. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 58. In some embodiments, the nucleic acid comprises a sequence as set forth in SEQ ID NO: 59. In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 59.

A summary of the elements in an exemplary logic gate and their nucleotide position is provided in Table 1. In some embodiments, the system comprises one or more elements provided in Table 1.

TABLE 1
Nucleotide Positions of Elements in an Exemplary Logic Gate System with
Homology Regions

| Element | Logic Gate 1 SEQ ID NO: 58 |
|---|---|
| sgRNA_94 | 1-20 |
| SEQ ID NO: 5 | |
| PAM sequence GGG | 21-23 |
| GS94 5′ homology | 24-473 |
| BGH poly A | 481-708 |
| SEQ ID NO: 6 | |
| 6X HNF1α response elements | 736-845 |
| YB-TATA promoter | 851-875 |
| CD8a signal peptide | 923-985 |
| SEQ ID NO: 8 | |
| CAR scFv | 986-1717 |
| SEQ ID NO: 32 | |
| AAA linker (amino acids AAA) | 1718-1726 |
| CD8a hinge | 1727-1861 |
| SEQ ID NO: 9 | |
| CD8a TM | 1862-1933 |
| SEQ ID NO: 10 | |
| 4-1BB | 1934-2059 |
| SEQ ID NO: 11 | |
| CD3zeta | 2060-2395 |
| SEQ ID NO: 12 | |
| WPRE | 2399-2990 |
| SEQ ID NO: 13 | |
| SV40 poly A | 3009-3140 |
| SEQ ID NO: 14 | |
| EF1a promoter | 3215-3588 |
| SEQ ID NO: 15 | |
| FAS & TGFBR2 shRNA module | 3589-4579 |
| intron | |
| SEQ ID NO: 20 | |
| CD8a signal peptide | 4628-4690 |
| SEQ ID NO: 8 | |
| Priming receptor scFv | 4691-5443 |
| SEQ ID NO: 44 | |
| CD8a hinge | 5447-5527 |
| SEQ ID NO: 21 | |
| Notch_TMD | 5528-5593 |
| SEQ ID NO: 22 | |
| Notch1_JMD | 5594-5722 |
| SEQ ID NO: 23 | |
| HNF1a DBD | 5723-6571 |
| SEQ ID NO: 24 | |
| TCR Linker | 6572-6580 |
| p65 | 6581-7153 |
| SEQ ID NO: 25 | |
| CD8a signal peptide (SPA) | 7232-7294 |
| SEQ ID NO: 8 | |
| CD34 epitope (SPA) | 7295-7342 |
| SEQ ID NO: 27 | |
| Cys linker (SPA) | 7343-7357 |
| SEQ ID NO: 28 | |
| gp130_TMD (SPA) | 7358-7423 |
| SEQ ID NO: 29 | |
| gp130_ICD (SPA) | 7424-8254 |
| SEQ ID NO: 30 | |
| human GH1 poly A | 8262-8740 |
| SEQ ID NO: 31 | |
| GS94 3′ homology | 8741-9190 |
| PAM sequence CCC | 9191-9193 |
| sgRNA 94 reverse complement | 9194-9213 |
| sequence | |

### Synthetic Receptors

In one aspect, synthetic receptors disclosed herein comprises an antigen binding domain that specifically binds Prostate Stem Cell Antigen (PSCA). In one aspect, synthetic receptors disclosed herein comprises an antigen binding domain that specifically binds (PSMA). In some embodiments, the receptors are isolated. In some embodiments, the domain is an extracellular domain. In some embodiments, the domain includes the ligand-binding portion of a receptor. In some embodiments, the domain includes an antigen-binding moiety that binds to one or more target antigens. In some embodiments, the antigen-binding moiety includes one or more antigen-binding determinants of an antibody or a functional antigen-binding fragment or equivalent thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), a VHH, and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the synthetic receptor is a chimeric antigen receptor (CAR) or a priming receptor.

### PSCA Receptors

In some embodiments, the antigen-binding domain specifically binds to Prostate Stem Cell Antigen (PSCA). In some embodiments, the antigen-binding domain includes an antigen-binding moiety that binds to Prostate Stem Cell Antigen (PSCA).

In some embodiments, provided herein are synthetic receptors comprising an antigen-binding domain that binds to Prostate Stem Cell Antigen (PSCA) (SEQ ID NO: 1).

In some embodiments, the synthetic receptor comprises any PSCA antibody or antigen binding fragments disclosed herein. In some embodiments, the isolated synthetic receptor comprises the sequence set forth in SEQ ID NO: 44. In some embodiments, the isolated synthetic receptor comprises an amino acid sequence encoded by the nucleic acid sequence as set forth in SEQ ID NO: 53.

In some embodiments, the isolated receptor's extracellular antigen-binding domains comprises the variable heavy (VH) chain sequence comprising three heavy chain CDR sequences, CDR-H1, CDR-H2, and CDR-H3, and the variable light (VL) chain sequence comprising three light chain CDR sequences, CDR-L1, CDR-L2, and CDR-L3 of any PSCA antibody or antigen binding fragments disclosed herein.

### PSMA Receptors

In some embodiments, the antigen-binding domain specifically binds to Prostate-Specific Membrane Antigen (PSMA). In some embodiments, the domain includes an antigen-binding moiety that binds to Prostate-Specific Membrane Antigen (PSMA).

In some embodiments, provided herein are synthetic receptors comprising an antigen-binding domain that binds to Prostate-Specific Membrane Antigen (PSMA) (SEQ ID NO: 2).

In some embodiments, the synthetic receptor comprises any PSMA antibody or antigen binding fragments disclosed herein. In some embodiments, the isolated synthetic receptor comprises the sequence as set forth in SEQ ID NO: 32. In some embodiments, the isolated synthetic receptor comprises an amino acid sequence encoded by the nucleic acid sequence as set forth in SEQ ID NO: 41.

In some embodiments, the isolated synthetic receptor's extracellular antigen-binding domains comprises the variable heavy (VH) chain sequence comprising three heavy chain CDR sequences, CDR-H1, CDR-H2, and CDR-H3, of any PSMA antibody or antigen binding fragments disclosed herein. In some embodiments, the isolated receptor's extracellular antigen-binding domains comprises the variable light (VL) chain sequence comprising three light chain CDR sequences, CDR-L1, CDR-L2, and CDR-L3 of any PSMA antibody or antigen binding fragments disclosed herein.

### Priming Receptors

Provided herein are priming receptors comprising an extracellular antigen-binding domain that specifically binds Prostate Stem Cell Antigen (PSCA). In some embodiments, the priming receptor comprises an extracellular antigen-binding domain that specifically binds Prostate Stem Cell Antigen (PSCA).

In certain aspects of the present disclosure, the priming receptor is a synthetic receptor based on the Notch protein. Binding of a natural Notch receptor to a cognate ligand, such as those from the Delta family of proteins, causes intramembrane proteolysis that cleaves an intracellular fragment of the Notch protein. This intracellular fragment is a transcriptional regulator that only functions when cleaved from Notch. Cleavage may occur by sequential proteolysis by ADAM metalloprotease and the gamma-secretase complex. This intracellular fragment enters the nucleus of a cell and activates cell-cell signaling genes. In contrast to a natural Notch protein, a synthetic notch priming receptor replaces the natural Notch intracellular fragment with one that causes a gene encoding a protein of choice, such as a CAR, to be transcribed upon release of the intracellular fragment from the priming receptor.

Notch receptors have a modular domain organization. The ectodomains of Notch receptors consist of a series of N-terminal epidermal growth factor (EGF)-like repeats that are responsible for ligand binding. In synthetic Notch receptors or priming receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of priming receptors can be used in the methods, cells, and nucleic acids as described herein. One type of priming receptor contemplated for use in the methods and cells herein comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Linker Notch” receptors comprise a heterologous extracellular ligand binding domain, a linking polypeptide lacking substantial sequence identity with a Notch receptor (e.g., a synthetic (GGS)n polypeptide sequence), a TMD, and an ICD. “Hinge Notch” receptors comprise a heterologous extracellular ligand binding domain, a hinge sequence comprising an oligomerization domain (i.e., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the priming receptor does not include a LIN-12-Notch repeat (LNR) and/or a heterodimerization domain (HD) of a Notch receptor.

### Priming Receptor Extracellular Domain

The priming receptor disclosed herein comprises an extracellular domain that specifically binds Prostate Stem Cell Antigen (PSCA). In some embodiments, provided herein are priming receptors comprising an extracellular antigen-binding domain that binds to Prostate Stem Cell Antigen (PSCA). In some embodiments, the priming receptor extracellular antigen-binding domains comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3 of any PSCA antibody or antigen binding fragments disclosed herein.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment or equivalent thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), a VHH, and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the extracellular antigen-binding domain specifically binds to Prostate Stem Cell Antigen. In some embodiments, the extracellular domain includes an antigen-binding moiety that binds to Prostate Stem Cell Antigen.

In some embodiments, the priming receptor comprises a PSCA antibody or antigen binding fragments disclosed herein. In some embodiments, the priming receptor binds to PSCA. In some embodiments, the priming receptor comprises a sequence at least 80%, 85%, 90%, 91%, 92%, 93%, 94%, 95%, 96%, 97%, 98%, 99%, or 100% identical to the sequence as set forth in SEQ ID NO: 44. In some embodiments, the priming receptor comprises the sequence set forth in SEQ ID NO: 44. In some embodiments, the priming receptor comprises a sequence with at least about 50%, 60%, 70%, 80%, 90%, 95%, 99%, or 100% sequence identity to the sequence as set forth in SEQ ID NO: 54. In some embodiments, the priming receptor is encoded by a nucleic acid sequence with at least about 50%, 60%, 70%, 80%, 90%, 95%, 99%, or 100% sequence identity to the sequence as set forth in SEQ ID NO: 55.

In various embodiments, a priming receptor comprises means for binding a PSCA protein, optionally binding a human PSCA protein in the region(s) of human PSCA bound by the PSCA binders (e.g., as described in the Examples below). In some embodiments, the means binds a PSCA protein. In some embodiments, the means binds a human PSCA protein. In some embodiments, the means is a PSCA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof). In some embodiments, the means for binding PSCA includes the anti-PSCA antibodies and antigen-binding fragments or equivalents thereof described herein.

The extracellular domain of the priming receptor can further comprise a signal sequence, such as a CD8α signal sequence. In some embodiments, the priming receptor comprises a CD8α signal sequence as set forth in SEQ ID NO: 8.

### Transmembrane Domain

In some embodiments, the priming receptor comprises a hinge domain. In some embodiments, the hinge domain is a CD8 or a CD8α hinge. In some embodiments, the priming receptor hinge domain comprises the sequence as set forth in SEQ ID NO: 21.

As described above, the priming receptor comprises a transmembrane domain (TMD) comprising one or more ligand-inducible proteolytic cleavage sites.

In some embodiments, the TMD comprises a Notch1 transmembrane domain.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014). Non limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one gamma secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, YASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the priming receptor comprises a Notch cleavage site, such as S2 or S3. Additional proteolytic cleavage sites suitable for the compositions and methods disclosed herein include, but are not limited to, ADAM10, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Yal-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO: 60), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 61), where cleavage occurs after the lysine residue. Another example of a protease cleavage site that can be included in a proteolytically cleavable linker is a thrombin cleavage site, e.g., Leu-Val-Pro-Arg (SEQ ID NO: 62). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

In some embodiments, the TMD domain comprises the sequence as set forth in SEQ ID NO: 22.

### Intracellular Domain

In some embodiments, the priming receptor comprises one or more intracellular domains from or derived from a transcriptional regulator and/or a DNA-binding domain. In some embodiments, the intracellular domain comprises means for modulating transcription of one or more genes. In some embodiments, the means for modulating transcription of one or more genes comprises a transcriptional regulator, e.g., a transcriptional regulator provided herein or an equivalent thereof. In some embodiments, the priming receptor comprises one or more intracellular domains from or derived from a transcriptional regulator and/or a DNA-binding domain. In some embodiments, the intracellular domain comprises an HNF1α/p65 domain or a Gal4/VP64 domain. In some embodiments, the priming receptor comprises one or more human or humanized intracellular domains.

Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” or “TAD” refers to the domain of a transcription factor that interacts with transcriptional control elements and/or transcriptional regulatory proteins (i.e., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (CAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. In some embodiments, the transcriptional regulator is p65. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

In some embodiments, the priming receptor comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNF1α, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cas6e, Casof, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu196, and TALENS. In some embodiments the DNA binding domain (DBD) is HNF1α.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

In some embodiments, the intracellular domain comprises the sequence as set forth in SEQ ID NOs: 24, 25, or 26. In some embodiments, the intracellular domain comprises an HNF1α DNA binding domain (DBD) sequence as set forth in SEQ ID NO: 24. In some embodiments, the intracellular domain comprises a p65 transcriptional activation domain (TAD) sequence as set forth in SEQ ID NO: 25. In some embodiments, the intracellular domain comprises an HNF1a/p65 DBD-TAD domain sequence as set forth in SEQ ID NO: 26.

### Juxtamembrane Domain

The ECD and the TMD, or the TMD and the ICD, can be linked to each other with a linking polypeptide, such as a juxtamembrane domain. “SynNotch” or synthetic notch receptors comprise a heterologous extracellular ligand-binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand-binding domain, a linking polypeptide lacking substantial sequence identity with a Notch receptor (for example, without limitation, having a synthetic (GGS) n polypeptide sequence), a TMD, and an ICD. “Hinge Notch” receptors comprise a heterologous extracellular ligand-binding domain, a hinge sequence comprising an oligomerization domain (i.e., a domain that promotes dimerization, trimerization, or higher order multimerization with a synthetic receptor and/or an existing host receptor), a TMD, and an ICD.

In some embodiments, the priming receptor comprises a juxtamembrane domain (JMD) peptide in between the extracellular domain and the transmembrane domain. In some embodiments, the priming receptor comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligomerization domain which promotes formation of dimers, trimers, or higher order assemblages of the receptor. Such JMD peptides are described in WO2021061872, hereby incorporated by reference in its entirety.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. The Minimal Linker sequence can be designed to include or omit a protease cleavage site, and can include or omit a glycosylation site or sites for other types of post-translational modification. In some embodiments, the Minimal Linker does not comprise a protease cleavage site or a glycosylation site.

In some embodiments, the priming receptor further comprises a hinge. Hinge linkers that can be used in the priming receptor can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs (SEQ ID NO: 63).

Hinge polypeptide sequences can also be derived from a CD8α hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8α hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Fn Notch linking polypeptide is derived from the Robo1 JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues. In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure.

### Stop-Transfer Sequence

In some embodiments, the priming receptor further comprises a stop-transfer sequence (STS) in between the transmembrane domain and the intracellular domains. The STS comprises a charged, lipophobic sequence. Without being bound by any theory, the STS serves as a membrane anchor, and is believed to prevent passage of the intracellular domain into the plasma membrane. The use of STS domains in priming receptors is described in WO2021061872, hereby incorporated by reference in its entirety. Non-limiting exemplary STS sequences include APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, LRP18, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKTFD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4 STS sequences. In some embodiments, the STS is heterologous to the transmembrane domain. In some embodiments, the STS is homologous to the transmembrane domain. STS sequences are described in WO2021061872, hereby incorporated by reference in its entirety.

In some embodiments, the STS domain comprises the sequence as set forth in SEQ ID NO: 23.

### Chimeric Antigen Receptors

In another aspect, provided herein are chimeric antigen receptors comprising an extracellular antigen-binding domain that specifically binds to Prostate-Specific Membrane Antigen (PSMA). The CAR can be a human CAR, comprising fully human sequences, e.g., natural human sequences.

In some embodiments, the chimeric antigen receptor includes an extracellular portion comprising an antigen binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some aspects, the chimeric antigen receptor includes an extracellular portion comprising an antigen binding domain described herein and an intracellular signaling domain. In some embodiments, an antibody or fragment includes an scFv, a VH, or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD8a or CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

### Chimeric Antigen Receptor Extracellular Domain

The chimeric antigen receptors disclosed herein comprises an extracellular domain that specifically binds Prostate-Specific Membrane Antigen (PSMA). In some embodiments, provided herein are chimeric antigen receptors comprising an extracellular antigen-binding domain that binds to Prostate-Specific Membrane Antigen (PSMA) (SEQ ID NO: 2). In some embodiments, the chimeric antigen receptors extracellular antigen-binding domains comprises a variable heavy (VH) chain sequence comprising three heavy chain CDR sequences, CDR-H1, CDR-H2, and CDR-H3. In some embodiments, the chimeric antigen receptors extracellular antigen-binding domains comprises a variable light (VL) chain sequence comprising three light chain CDR sequences, CDR-L1, CDR-L2, and CDR-L3 of any PSMA antibody or antigen binding fragments disclosed herein.

In some embodiments, the chimeric antigen receptor binds to PSMA. In some embodiments, the chimeric antigen receptor comprises a sequence at least 80%, 85%, 90%, 91%, 92%, 93%, 94%, 95%, 96%, 97%, 98%, 99%, or 100% identical to the sequence as set forth in SEQ ID NO: 32. In some embodiments, the chimeric antigen receptor comprises the sequence as set forth in SEQ ID NO: 32. In some embodiments, the CAR comprises a sequence with at least about 50%, 60%, 70%, 80%, 90%, 95%, 99%, or 100% sequence identity to the sequence as set forth in SEQ ID NO: 42. In some embodiments, the CAR is encoded by a nucleic acid sequence with at least about 50%, 60%, 70%, 80%, 90%, 95%, 99%, or 100% sequence identity to the sequence as set forth in SEQ ID NO: 43.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment or equivalent thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), a VHH, and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the extracellular antigen-binding domain specifically binds to Prostate-Specific Membrane Antigen (PSMA). In some embodiments, the extracellular domain includes an antigen-binding moiety that binds to Prostate-Specific Membrane Antigen (PSMA).

In various embodiments, a CAR comprises means for binding a PSMA protein, optionally binding a human PSMA protein in the region(s) of human PSMA bound by the PSMA binders (e.g., as described in the Examples below). In some embodiments, the means binds a PSMA protein. In some embodiments, the means binds a human PSMA protein. In some embodiments, the means is a PSMA antibody or antigen-binding fragment or equivalent thereof (e.g., a full length antibody or a F(ab′)2 fragment, a Fab fragment, a single chain variable fragment (scFv), and a single domain antibody (sdAb), or a functional fragment thereof) means for binding a PSMA protein. In some embodiments, the means for binding PSMA includes the anti-PSMA antibodies and antigen-binding fragments or equivalents thereof described herein.

The extracellular domain of the CAR can further comprise a signal sequence, such as a CD8α signal sequence. In some embodiments, the CAR comprises a CD8α signal sequence as set forth in SEQ ID NO: 8.

### CAR Transmembrane Domain

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain (TMD) of the receptor, e.g., the CAR, is a transmembrane domain of human CD28 or variant thereof, e.g., a 27-amino acid transmembrane domain of a human CD28 (UniProt Accession No.: P10747).

In some embodiments, the transmembrane domain (TMD) of the receptor, e.g., the CAR, is a transmembrane domain of human CD8α or variant thereof, e.g., a 24-amino acid transmembrane domain of a human CD8α (UniProt Accession No.: P01732).

In some embodiments, the CAR comprises a CD8α or CD28 TMD. In some embodiments, the CD8α TMD comprises the sequence set forth in SEQ ID NO: 10.

### CAR Hinge

In some embodiments, the CAR further includes a spacer, which may be or include at least a portion of an immunoglobulin constant region or variant or modified version thereof, such as a hinge region, e.g., a CD8a hinge, a CD28 hinge, an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include CD8a hinge, CD28 hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the CAR hinge comprises a CD8α, truncated CD8α, or CD28 hinge domain. In some embodiments, the hinge domain comprises a CD8α hinge comprising the sequence as set forth in SEQ ID NO: 9.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

### CAR Intracellular Domain

In some embodiments, upon ligation of the CAR, the cytoplasmic domain or intracellular signaling domain of the receptor activates at least one of the normal effector functions or responses of the immune cell, e.g., T cell engineered to express the receptor. In some embodiments, the CAR comprises means for activating at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the means for at least one of the normal effector functions or responses of the immune cell comprises a CAR intracellular activation domain, e.g., an intracellular activation domain provided herein or an equivalent thereof. In some embodiments, the means for at least one of the normal effector functions or responses of the immune cell comprises a CAR intracellular activation domain and a CAR co-stimulatory domain, e.g., a co-stimulatory domain provided herein or an equivalent thereof.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16.

In some embodiments, the intracellular signaling domain comprises a human CD3 zeta stimulatory signaling domain or functional variant thereof, such as a 112 AA cytoplasmic domain of isoform 3 of human CD3 zeta. (UniProt Accession No.: P20963.2) or a CD3 zeta signaling domain as described in U.S. Pat. No. 7,446,190 or U.S. Pat. No. 8,911,993.

In some embodiments, cytoplasmic signaling molecule(s) in the CAR contain(s) a cytoplasmic signaling domain, portion thereof, or sequence derived from CD3 zeta. In some embodiments, the intracellular activation domain comprises a CD32 domain. In some embodiments, the intracellular activation domain comprises a CD33 domain set forth in SEQ ID NO: 12.

In some embodiments, the intracellular domain comprises an intracellular costimulatory signaling domain of 41BB or functional variant or portion thereof, such as a 42-amino acid cytoplasmic domain of a human 4-1BB (UniProt Accession No. Q07011.1) or functional variant or portion thereof.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB. In some embodiments, the chimeric antigen receptor contains an intracellular domain of a T cell costimulatory molecule. In some aspects, the T cell costimulatory molecule is 4-1BB.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD8a transmembrane and signaling domain linked to a CD3 (e.g., CD3-zeta) intracellular domain. In some embodiments, the intracellular signaling domain comprises a 4-1BB (CD137, TNFRSF9) co-stimulatory domains, linked to a CD3 zeta intracellular domain. In some embodiments, the CAR comprises a 4-1BB co-stimulatory domain. In some embodiments, the 4-1BB co-stimulatory domain comprises the sequence as set forth in SEQ ID NO: 11.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,γ-diaminopropionic acid, homophenylalanine, and α-tertbutylglycine.

For example, in some embodiments, the CAR includes an antibody or fragment thereof, including single chain antibodies (sdAbs, e.g. containing only the VH region, also called a VHH), VH domains, and scFvs, described herein, a spacer such as a CD8a hinge, a CD8a transmembrane domain, a 4-1BB intracellular signaling domain, and a CD3 zeta signaling domain. In some embodiments, the CAR includes an antibody or fragment, including sdAbs and scFvs described herein, a spacer such as a CD8a hinge, a CD8a transmembrane domain, a 4-1BB intracellular signaling domain, and a CD3 zeta signaling domain.

Transgenes expressing the priming receptor and CAR system may be introduced into cells, such as a T cell, using, for example, a site-specific technique. With site specific integration of the transgenes (e.g. priming receptor and CAR), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered cells have applications to immune-oncology. The priming receptor and CAR, for example, can be selected to target different specific tumor antigens. Examples of cancers that can be effectively targeted using such cells are blood cancers or solid cancers. In some embodiments, immune cell therapy can be used to treat solid tumors.

### Synthetic Pathway Activators

In various aspects, systems disclosed herein employ one or more “synthetic pathway activators” (SPAs). CAR-expressing immune cells can be limited by the necessity for in vivo expansion following infusion. To achieve robust expansion, T cells require three signals: antigen-stimulation, co-stimulation, and cytokine-induced stimulation. Activation of CARs is sufficient to induce the first two signals, but cannot recapitulate cytokine signaling. Furthermore, the tumor microenvironment is often immunosuppressive and devoid of pro-inflammatory cytokines. SPAs can thus be used to stimulate robust in vivo expansion and enhance desirable properties (i.e., increased survival, persistence, and potency) of T cells such as those expressing priming receptors and/or CARs as described herein.

### SPA Structure

SPAs mimic activation of interleukin signaling. Interleukin receptors are cytokine receptors that signal through Signal Transducer and Activator of Transcription (STAT) transcription factors (e.g., STAT1, STAT3 and STAT5). Interleukin receptors typically function by dimerization in response to ligand binding. Once dimerized, receptors can bind janus-associated kinases (JAKs) to induce JAK cross-phosphorylation and downstream “JAK/STAT” signaling. Accordingly, induced receptor agonism or ligand-independent dimerization of receptors can be utilized to induce constitutive receptor activity and thus, constitutive cytokine signaling.

In some embodiments, the SPA comprises a leucine zipper-gp130 (referred to herein as “L-gp130”) or an L-gp130 intracellular signaling domain. L-gp130 comprises a homodimer, with each monomer comprising (a) an extracellular domain comprising an inserted cysteine residue that forms a disulfide linkage with another monomer and a c-Jun leucine zipper; and (b) a gp130 transmembrane domain and intracellular signaling domain. The cysteine residue and the leucine zipper on each polypeptide can induce the formation of stable homodimers that mimic constitutive IL-6R activation. Additional details on the construction of L-gp130 are described in Stuhlmann-Laeisz et al. Mol Biol Cell. 2006 July; 17(7):2986-95 and in WO2020200325, which are hereby incorporated by reference in their entirety.

Additional exemplary SPAs are provided in International Patent Application No: WO2024192100 A1, published on Sep. 19, 2024, and U.S. Patent Application 20250270288 A1, both of which are hereby incorporated by reference in their entirety.

In various embodiments, SPAs comprise interleukin receptors or functional fragments thereof. In some embodiments, SPAs comprise or are derived from interleukin receptor intracellular signaling domains or functional fragments thereof. In some embodiments, SPAs comprise or are derived from glycoprotein 130 (gp130) (interleukin-6 signal transducer (IL6ST)) polypeptides or functional fragments thereof. Interleukin-6 signal transducer (IL6ST) is also known as glycoprotein 130 (gp130) and Interleukin-6 receptor subunit beta (UNIPROT P40189).

In various embodiments, one or more structural alterations can be made to confer constitutive activity to a SPA or functional fragment thereof. In some embodiments, structures or mutations can be added to induce SPA multimerization. In some embodiments, one or more amino acids can be mutated to a cysteine to allow formation of one or more disulfide bond(s), e.g., between two receptor monomers. In some embodiments, one or more amino acids can be inserted into a wild-type receptor polypeptide to promote dimerization, e.g., through formation of one or more disulfide bond(s).

In some embodiments, an exogenous polypeptide is operatively linked to a cytokine receptor or functional fragment thereof to cause their multimerization. In some embodiments, a leucine zipper polypeptide is operatively linked to a cytokine receptor or functional fragment thereof. In some embodiments, the leucine zipper polypeptide is a c-Jun leucine zipper. In some embodiments, an exogenous scaffold is operatively linked to a cytokine receptor or functional fragment thereof (e.g., gp130).

In various embodiments, SPAs are anchored to the cellular membrane. In some embodiments SPAs comprise an extracellular domain, a transmembrane domain, and an intracellular signaling domain. In some embodiments, SPAs comprise a transmembrane domain of an interleukin receptor.

In some embodiments, the SPA comprises an extracellular domain comprising a CD34 epitope or CD34 extracellular domain or fragment thereof. In some embodiments, the SPA comprises an extracellular domain comprising a CD34 epitope, an unpaired cysteine residue for multimerization, a gp130 transmembrane domain, and a gp130 intracellular domain. In some embodiments, the SPA comprises a sequence as set forth in SEQ ID NO: 56. The gp130 transmembrane domain nucleic acid sequence is provided in SEQ ID NO: 29. In some embodiments, the SPA comprises a gp130 transmembrane domain sequence as set forth in SEQ ID NO: 29. The gp130 intracellular signaling domain sequence is provided in SEQ ID NO: 30. In some embodiments, the SPA comprises a gp130 intracellular signaling domain sequence as set forth in SEQ ID NO: 30. The CD34 epitope nucleic acid sequence is set forth in SEQ ID NO: 27. In some embodiments, the SPA comprises an extracellular domain comprising the nucleic acid sequence as set forth in SEQ ID NO: 27. In some embodiments, the SPA comprises a nucleic acid as set forth in SEQ ID NOs: 57.

### Nucleic Acids and Vectors

In another aspect, provided herein are one or more nucleic acids, wherein the one or more nucleic acids encode one or more polypeptides described herein. In certain embodiments, the encoded polypeptide is one or more of an antibody or antigen binding fragment thereof, priming receptor (primeR), chimeric antigen receptor (CAR), or synthetic pathway activator (SPA) described herein. In another aspect, provided herein are nucleic acids encoding a system. In certain aspects the system comprises: a first chimeric polypeptide comprises a priming receptor comprising a first extracellular antigen-binding domain that specifically binds Prostate Stem Cell Antigen (PSCA); a second chimeric polypeptide comprising a chimeric antigen receptor (CAR) comprising a second extracellular antigen-binding domain that specifically binds to Prostate-Specific Membrane Antigen (PSMA); an optional third chimeric polypeptide comprising a synthetic pathway activator (SPA); and at least one or more nucleic acids comprising a nucleic acid sequence at least 15 nucleotides in length complementary to: a nucleic acid encoding human Fas Cell Surface Death Receptor (FAS) comprising the sequence set forth in SEQ ID NO: 3; and a nucleic acid encoding human Transforming Growth factor (TGF)-β Receptor 2 (TGFBR2) comprising the sequence set forth in SEQ ID NO: 4. In some embodiments, the nucleic acid sequence at least 15 nucleotides in length is complementary to nucleotides 1126 to 1364 of a nucleic acid encoding human FAS comprising the sequence set forth in SEQ ID NO: 3.

### RNA Interference Molecules

Transforming Growth Factor Beta Receptor 2 (TGF-βR2 or TGFBR2; HGNC: 11773, NCBI Entrez Gene: 7048, UniProtKB/Swiss-Prot: P37173) is a transmembrane serine/threonine protein kinase and forms a heterodimeric complex with TGF-beta receptor type-1 (TGFBR1) when bound to TGF-beta, resulting in transduction of the TGF-beta signal from the cell surface to the cytoplasm.

Fas Cell Surface Death Receptor (or Fas Receptor, FAS, CD95, or TNFRSF6; HGNC: 11920, NCBI Entrez Gene: 355; UniProtKB/Swiss-Prot: P25445) is an apoptosis-inducing TNF receptor superfamily member.

As used herein, “target gene” refers to a nucleic acid sequence in a cell, wherein the expression of the sequence may be specifically and effectively modulated using the nucleic acid molecules and methods described herein. In certain embodiments, the target gene may be implicated in the growth (proliferation), maintenance (survival), and/or immune behavior of an individual's immune cells. In some embodiments, the target gene is FAS. In some embodiments, one or more nucleic acid molecules target the FAS gene. In some embodiments, the target gene is Transforming Growth Factor Beta Receptor 2 (TGFBR2). In some embodiments, two or more nucleic acid molecules target the TFGBR2 gene. In some embodiments, three or more nucleic acid molecules target the TFGBR2 gene. In some embodiments, more than one target gene is modulated using a nucleic acid molecule and methods described herein. In some embodiments, at least two target gene are modulated using the nucleic acid molecules and methods described herein. In some embodiments, the nucleic acid molecule(s) is an shRNA. In some embodiments, the target genes are at least FAS and TGFBR2.

In one aspect, provided herein are nucleic acid comprising a nucleic acid sequence at least 15 nucleotides in length complementary to a nucleic acid encoding human Transforming Growth Factor Beta Receptor 2 (TGFBR2) (SEQ ID NO: 4). In some embodiments, the nucleic acid comprises a nucleic acid sequence at least 15 nucleotides in length complementary to nucleotides 2215-2236, 4430-4451, or 3761-3782 of a nucleic encoding human Transforming Growth Factor Beta Receptor 2 (TGFBR2) comprising the sequence set forth in SEQ ID NO: 4.

Additional exemplary nucleic acid sequence at least 15 nucleotides in length complementary to 15-40 nucleotides of a nucleic acid encoding human Transforming Growth Factor Beta Receptor 2 (TGFBR2) and/or human Fas Cell Surface Death Receptor (FAS) are provided in International Patent Applications WO2024059618A1 and WO2023064928A1, both of which are hereby incorporated by reference in their entirety.

In one aspect, provided herein are nucleic acid comprising a nucleic acid sequence at least 15 nucleotides in length complementary a nucleic acid encoding human Transforming Growth Factor Beta Receptor 2 (TGFBR2) (SEQ ID NO: 4), wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human Transforming Growth Factor Beta Receptor 2 (TGFBR2) (SEQ ID NO: 4). In some embodiments, the nucleic acid sequence at least 15 nucleotides in length is complementary to 15-50 nucleotides of TGFBR2. In some embodiments, the nucleic acid comprises at least 80%, 85%, 90%, 91%, 92%, 93%, 94%, 95%, 96%, 97%, 98%, 99%, or 100% identity to a sequence selected from the group consisting of the sequences set forth in SEQ ID NOs: 17-19. In some embodiments, the nucleic acid comprises at least two sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 17-19. In some embodiments, the nucleic acid comprises at least three sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 17-19. In some embodiments, the nucleic acid comprises a sequence with at least 80%, 85%, 90%, 91%, 92%, 93%, 94%, 95%, 96%, 97%, 98%, 99%, or 100% identity to the sequence set forth in SEQ ID NO: 17. In some embodiments, the nucleic acid comprises a sequence with at least 80%, 85%, 90%, 91%, 92%, 93%, 94%, 95%, 96%, 97%, 98%, 99%, or 100% identity to the sequence set forth in SEQ ID NO: 18. In some embodiments, the nucleic acid comprises a sequence with at least 80%, 85%, 90%, 91%, 92%, 93%, 94%, 95%, 96%, 97%, 98%, 99%, or 100% identity to the sequence set forth in SEQ ID NO: 19.

In one aspect, provided herein are nucleic acids comprising a nucleic acid sequence at least 15 nucleotides in length complementary to a nucleic acid encoding human FAS (SEQ ID NO: 3) comprising the sequence set forth in SEQ ID NO: 3, wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human FAS comprising the sequence set forth in SEQ ID NO: 3. In some embodiments, the nucleic acid comprises a nucleic acid sequence at least 15 nucleotides in length complementary to a nucleic acid encoding human Fas Cell Surface Death Receptor (FAS) comprising the sequence set forth in SEQ ID NO: 3. In some embodiments, the nucleic acid sequence at least 15 nucleotides in length is complementary to 15-50 nucleotides of FAS. In some embodiments, the nucleic acid comprises a sequence as set forth in SEQ ID NO: 16. In some embodiments, the nucleic acid sequence is complementary to nucleotides 1126 to 1364 of a nucleic acid encoding human FAS comprising the sequence set forth in SEQ ID NO: 3.

In some embodiments, the nucleic acid comprises at least 80%, 85%, 90%, 91%, 92%, 93%, 94%, 95%, 96%, 97%, 98%, 99%, or 100% identity to a sequence as set forth in SEQ ID NO: 16.

In some embodiments, the nucleic acid comprises a sequence as set forth in SEQ ID NO: 16 In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 16.

In some embodiments, the nucleic acid comprises a sequence as set forth in SEQ ID NO: 20 In some embodiments, the nucleic acid comprises a sequence at least 80%, 85%, 90%, 91%, 92%, 93%, 94%, 95%, 96%, 97%, 98%, 99%, or 100% identical to a sequence as set forth in SEQ ID NO: 20.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 16 and a passenger sequence complementary to SEQ ID NO: 16.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 17 and a passenger sequence complementary to SEQ ID NO: 17.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 18 and a passenger sequence complementary to SEQ ID NO: 18.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 19 and a passenger sequence complementary to SEQ ID NO: 19.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 17 and a passenger sequence complementary to SEQ ID NO: 17, a sequence at least 80%, 85%, 90%, 91%, 92%, 93%, 94%, 95%, 96%, 97%, 98%, 99%, or 100% identical to the nucleic acid sequence of SEQ ID NO: 18 and a passenger sequence complementary to SEQ ID NO: 18; and a sequence at least 80%, 85%, 90%, 91%, 92%, 93%, 94%, 95%, 96%, 97%, 98%, 99%, or 100% identical to the nucleic acid sequence of SEQ ID NO: 19 and a passenger sequence complementary to SEQ ID NO: 19.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 17 and a passenger sequence complementary to SEQ ID NO: 17, and a sequence at least 80%, 85%, 90%, 91%, 92%, 93%, 94%, 95%, 96%, 97%, 98%, 99%, or 100% identical to the nucleic acid sequence of SEQ ID NO: 18 and a passenger sequence complementary to SEQ ID NO: 18. In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 17 and a passenger sequence complementary to SEQ ID NO: 17, and a sequence at least 80%, 85%, 90%, 91%, 92%, 93%, 94%, 95%, 96%, 97%, 98%, 99%, or 100% identical to the nucleic acid sequence of SEQ ID NO: 19 and a passenger sequence complementary to SEQ ID NO: 19. In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 18 and a passenger sequence complementary to SEQ ID NO: 18, and a sequence at least 80%, 85%, 90%, 91%, 92%, 93%, 94%, 95%, 96%, 97%, 98%, 99%, or 100% identical to the nucleic acid sequence of SEQ ID NO: 19 and a passenger sequence complementary to SEQ ID NO: 19.

In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 16 and a passenger sequence complementary to SEQ ID NO: 16, and a sequence at least 80%, 85%, 90%, 91%, 92%, 93%, 94%, 95%, 96%, 97%, 98%, 99%, or 100% identical to the nucleic acid sequence of SEQ ID NO: 17 and a passenger sequence complementary to SEQ ID NO: 17. In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 16 and a passenger sequence complementary to SEQ ID NO: 16, and a sequence at least 80%, 85%, 90%, 91%, 92%, 93%, 94%, 95%, 96%, 97%, 98%, 99%, or 100% identical to the nucleic acid sequence of SEQ ID NO: 18 and a passenger sequence complementary to SEQ ID NO: 18. In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 16 and a passenger sequence complementary to SEQ ID NO: 16, and a sequence at least 80%, 85%, 90%, 91%, 92%, 93%, 94%, 95%, 96%, 97%, 98%, 99%, or 100% identical to the nucleic acid sequence of SEQ ID NO: 19 and a passenger sequence complementary to SEQ ID NO: 19. In some embodiments, the nucleic acid sequence comprises a sequence at least 80%, 85%, 90%, 91%, 92%, 93%, 94%, 95%, 96%, 97%, 98%, 99%, or 100% identical to the nucleic acid sequence of SEQ ID NO: 16 and a passenger sequence complementary to SEQ ID NO: 16; a sequence at least 80%, 85%, 90%, 91%, 92%, 93%, 94%, 95%, 96%, 97%, 98%, 99%, or 100% identical to the nucleic acid sequence of SEQ ID NO: 17 and a passenger sequence complementary to SEQ ID NO: 17; a sequence at least 80%, 85%, 90%, 91%, 92%, 93%, 94%, 95%, 96%, 97%, 98%, 99%, or 100% identical to the nucleic acid sequence of SEQ ID NO: 18 and a passenger sequence complementary to SEQ ID NO: 18; and a sequence at least 80%, 85%, 90%, 91%, 92%, 93%, 94%, 95%, 96%, 97%, 98%, 99%, or 100% identical to the nucleic acid sequence of SEQ ID NO: 19 and a passenger sequence complementary to SEQ ID NO: 19.

In some embodiments, the nucleic acid is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid is capable of reducing expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid is capable of reducing expression of TGFBR2 and/or FAS in the immune cell by between about 50-100%, 50-99%, 50-95%, 50-90%, 50-85%, 50-80%, 50-75%, 50-70%, 50-65%, 50-60%, 50-55%, or as compared to a control cell that does not comprise the nucleic acid molecule(s).

In some embodiments, the nucleic acid sequence complementary to FAS and/or TGFBR2 is at least 15, 16, 17, 18, 19, 20, 21, or 22 nucleotides in length. In some embodiments, the nucleic acid sequence complementary to FAS and/or TGFBR2 is no more than or up to 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the nucleic acid sequence is complementary to 15-50 nucleotides of TGFBR2 or FAS. In some embodiments, the nucleic acid sequence is complementary to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of TGFBR2. In some embodiments, the nucleic acid sequence comprises at least one, two, and/or three nucleic acid sequences that are complementary to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of TGFBR2. In some embodiments, the nucleic acid sequence is complementary to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of FAS. In some embodiments, the at least one, two, and/or three nucleic acid sequences that are at least 15 nucleotides in length complementary to 15-50 nucleotides of TGFBR2 are 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the nucleic acid sequence at least 15 nucleotides in length complementary to 15-50 nucleotides of FAS is 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the nucleic acid sequence comprises a first nucleic acid sequence complementary to 15-50 nucleotides of FAS, and a second, third, and/or fourth nucleic acid sequence complementary to 15-50 nucleotides of TGFBR2. In some embodiments, the first nucleic acid sequence is complementary to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of FAS. In some embodiments, the second, third, and/or fourth nucleic acid sequence is complementary to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of TGFBR2. In some embodiments, the nucleic acid sequence at least 15 nucleotides in length complementary to 15-50 nucleotides of FAS is 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the nucleic acid sequence at least 15 nucleotides in length complementary to 15-50 nucleotides of TGFBR2 is 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the nucleic acid is an RNA interference (RNAi) molecule. Exemplary RNAi molecules include short hairpin RNA (shRNA), a small interfering RNA (siRNA), a double stranded RNA (dsRNA), or an antisense oligonucleotide. In some embodiments, the nucleic acid is a short hairpin RNA (shRNA), a small interfering RNA (siRNA), a double stranded RNA (dsRNA), or an antisense oligonucleotide. In some embodiments, the nucleic acid is an shRNA.

In some embodiments, the nucleic acid sequence is the guide sequence of an shRNA. In some embodiments, the nucleic acid sequence further comprises a nucleic acid sequence complementary to the guide sequence comprising the passenger sequence of an RNAi molecule such as an siRNA or shRNA. In some embodiments, the nucleic acid sequence further comprises a loop nucleic acid sequence between the guide sequence and the complementary passenger sequence. In some embodiments, the nucleic acid sequence comprises the guide sequence of an shRNA, a loop sequence, and the complementary passenger sequence. In some embodiments, the nucleic acid sequence comprises, in a 5′ to 3′ direction, the guide sequence, the loop sequence, and the complementary passenger sequence. In some embodiments, the nucleic acid sequence comprises, in a 5′ to 3′ direction, the complementary passenger sequence, the loop sequence, and the guide sequence. In some embodiments, the guide sequence comprises any one of the sequences as set forth in SEQ ID NO: 16, 17, 18, and/or 19. In some embodiments, the nucleic acid sequence comprises each of the guide sequences as set forth in SEQ ID NO: 16, 17, 18, and/or 19.

Single-stranded hairpin ribonucleic acids (shRNAs) are short duplexes where the sense and antisense strands are linked by a hairpin loop. They consist of a stem-loop structure that can be transcribed in cells from an RNA polymerase II or RNA polymerase III promoter on a plasmid construct. Once expressed, shRNAs are processed into RNAi species. Expression of shRNA from a plasmid is known to be relatively stable, thereby providing strong advantages over, for example, the use of synthetic siRNAs. shRNA expression units may be incorporated into a variety of plasmids, liposomes, viral vectors, and other vehicles for delivery and integration into a target cell. Expression of shRNA from a plasmid can be stably integrated for constitutive expression. shRNAs are synthesized in the nucleus of cells, further processed and transported to the cytoplasm, and then incorporated into the RNA-induced silencing complex (RISC) for activity. The shRNAs are converted into active siRNA molecules (which are capable of binding to, sequestering, and/or preventing the translation of mRNA transcripts encoded by target genes).

The Argonaute family of proteins is the major component of RISC. Within the Argonaute family of proteins, only Ago2 contains endonuclease activity that is capable of cleaving and releasing the passenger strand from the stem portion of the shRNA molecule. The remaining three members of Argonaute family, Ago1, Ago3 and Ago4, which do not have identifiable endonuclease activity, are also assembled into RISC and are believed to function through a cleavage-independent manner. Thus, RISC can be characterized as having cleavage-dependent and cleavage-independent pathways.

RNAi (e.g., antisense RNA, siRNA, microRNA, shRNA, etc.) are described in International Publication Nos. WO2018232356A1, WO2019084552A1, WO2019226998A1, WO2020014235A1, WO2020123871A1, and WO2020186219A1, each of which is herein incorporated by reference for all purposes.

Antisense oligonucleotide structure and chemical modifications are described in International PCT Publication No. WO20/132521, which is hereby incorporated by reference.

dsRNA and shRNA molecules and methods of use and production are described in U.S. Pat. Nos. 8,829,264; 9,556,431; and 8,252,526, each of which are hereby incorporated by reference

siRNA molecules and methods of use and production are described in U.S. Pat. No. 7,361,752 and US Patent Application No. US20050048647, both of which are hereby incorporated by reference.

Additional methods and compositions for RNA interference such as shRNA, siRNA, dsRNA, and antisense oligonucleotides are generally known in the art, and are further described in U.S. Pat. Nos. 7,361,752; 8,829,264; 9,556,431; 8,252,526, International PCT Publication No. WO00/44895; International PCT Publication No. WO01/36646; International PCT Publication No. WO99/32619; International PCT Publication No. WO00/01846; International PCT Publication No. WO01/29058; and International PCT Publication No. WO00/44914; International PCT Publication No. WO04/030634; each of which are hereby incorporated by reference.

The nucleic acid sequences (or constructs) that may be used to encode the RNAi molecules, such as an shRNA described herein, may comprise a promoter, which is operably linked (or connected), directly or indirectly, to a sequence encoding the RNAi molecules. Such promoters may be selected based on the host cell and the effect sought. Non-limiting examples of suitable promoters include constitutive and inducible promoters, such as EF1α or inducible Hepatocyte Nuclear Factor 1α(HNF1α)-YB TATA or RNA polymerase II (pol II)-based promoters. In some embodiments, the constitutive promoter is EF1a. In some embodiments, the EF1a promoter comprises as sequence as set forth in SEQ ID NO: 15. Non-limiting examples of suitable promoters further include the tetracycline inducible or repressible promoter, RNA polymerase I or III-based promoters, the pol II dependent viral promoters, such as the CMV-IE promoter, and the pol III U6 and H1 promoters, as well as Hepatocyte Nuclear Factor 1α (HNF1α)-YB TATA promotor provided in SEQ ID NO: 7. The bacteriophage T7 promoter may also be used (in which case it will be appreciated that the T7 polymerase must also be present). The nucleic acid sequences need not be restricted to the use of any single promoter, especially since the nucleic acid sequences may comprise two or more shRNAs (i.e., a combination of effectors), including but not limited to incorporated shRNA molecules. Each incorporated promoter may control one, or any combination of, the shRNA molecule components.

In certain embodiments, the promoter may be preferentially active in the targeted cells, e.g., it may be desirable to preferentially express at least one nucleic acid in immune cells using an immune cell-specific promoter. Introduction of such constructs into host cells may be effected under conditions whereby the two or more nucleic acids that are contained within the nucleic acid precursor transcript initially reside within a single primary transcript, such that the separate RNA molecules (for example, shRNA each comprising its own stem-loop structure) are subsequently excised from such precursor transcript by an endogenous ribonuclease. The resulting mature nucleic acids (e.g., shRNAs) may then induce degradation, and/or translation repression, of target gene mRNA transcripts produced in the cell. Alternatively, each of the precursor stem-loop structures may be produced as part of a separate transcript, in which case each nucleic acid sequence will preferably include its own promoter and transcription terminator sequences. Additionally, the multiple nucleic acid precursor transcripts may reside within a single primary transcript.

The stem-loop structures of the shRNA nucleic acids described herein may be about 40 to 100 nucleotides long or, preferably, about 50 to 75 nucleotides long. The stem region may be about 15-45 nucleotides in length (or more), or about 20-30 nucleotides in length. In some embodiments, the stem region is 22 nucleotides in length. In some embodiments, the stem region is no more than, or up to, 15, 16, 17, 18, 19, 20, 21, 22, 23, 24, 25, 26, 27 28 29, 30, 31, 32, 33, 34, 35, 36, 37, 38, 39, 40, 41, 42, 43, 44, or 45 nucleotides in length.

The stem may comprise a perfectly complementary duplex (but for any 3′ tail), however, bulges or interior loops may be present on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. The loop portion will preferably be 6-15 nucleotides in size.

As described herein, the stem regions of the shRNAs comprise passenger strands and guide strands, whereby the guide strands contain sequences complementary to the target mRNA transcript encoded by the target gene(s). Preferably, the G-C content and matching of guide strand and passenger strand is carefully designed for thermodynamically-favorable strand unwind activity with or without endonuclease cleavage. Furthermore, the specificity of the guide strand is preferably confirmed via a BLAST search (www.ncbi.nim.nih.gov/BLAST).

The disclosure herein provides that the expression level of multiple target genes may be modulated using the methods and nucleic acids described herein. For example, the disclosure herein provides that a first set of nucleic acids may be designed to include a sequence (a guide strand) that is designed to reduce the expression level of a first target gene, whereas a second set of nucleic acids may be designed to include a sequence (a guide strand) that is designed to reduce the expression level of a second target gene. The different sets of nucleic acids may be expressed and reside within the same, or separate, preliminary transcripts. In certain embodiments, such multiplex approach, i.e., the use of the nucleic acids described herein to modulate the expression level of two or more target genes, may have an enhanced therapeutic effect on a patient. For example, if a patient is provided with cells expressing the nucleic acid molecules described herein to treat, prevent, or ameliorate the effects of cancer, it may be desirable to provide the patient with two or more types of nucleic acid molecules, which are designed to reduce the expression level of multiple genes that are implicated in activation or repression of immune cells.

The nucleic acid molecule(s) may be chemically synthesized, or in vitro transcribed, and may further include one or more modifications to phosphate-sugar backbone or nucleosides residues.

Other methods known in the art for introducing nucleic acids to cells may be used, such as lipid-mediated carrier transport, chemical mediated transport, such as calcium phosphate, and the like. Thus, the nucleic acid molecule(s) construct may be introduced along with components that perform one or more of the following activities: enhance RNA uptake by the cell, promote annealing of the duplex strands for shRNA, stabilize the annealed shRNA strands, or otherwise increase inhibition of the target gene.

### Additional Elements

In some embodiments, the one or more nucleic acid(s) further comprises a 5′ homology directed repair arm and/or a 3′ homology directed repair arm complementary to an insertion site in a host cell chromosome. In some embodiments, the one or more nucleic acid(s) comprises the 5′ homology directed repair arm and the 3′ homology directed repair arm. In some embodiments, the one or more nucleic acid(s) is incorporated into a vector. In certain embodiments, the vector is a nanoplasmid. In certain embodiments, the vector is an expression cassette or an expression vector. In some embodiments, the expression cassette or the expression vector further comprises a constitutive promoter upstream of the one or more nucleic acid(s).

In some embodiments, the priming receptor, CAR, SPA, and/or the RNA interference nucleic acid are incorporated into a single expression cassette or a single expression vector. In some embodiments, the priming receptor, CAR, SPA, and/or the RNA interference nucleic acid are incorporated into two or more expression cassettes or expression vectors. In some embodiments, the expression vector(s) is a non-viral vector.

In some embodiments, the expression cassette or expression vector comprises a sequence as set forth in SEQ ID NO: 58. In some embodiments, the expression cassette comprises a sequence at least 80%, 85%, 90%, 91%, 92%, 93%, 94%, 95%, 96%, 97%, 98%, 99%, or 100% identical to a sequence as set forth in SEQ ID NO: 58. In some embodiments, the expression cassette or expression vector comprises a sequence as set forth in SEQ ID NO: 59. In some embodiments, the expression cassette comprises a sequence at least 80%, 85%, 90%, 91%, 92%, 93%, 94%, 95%, 96%, 97%, 98%, 99%, or 100% identical to a sequence as set forth in SEQ ID NO: 59.

The one or more interfering nucleic acid sequences (e.g., one or more shRNA) can be encoded in the intron regions of the nucleic acid insert, DNA template, module, cassette, single expression cassette, or a single expression vector that also encodes the priming receptor, CAR, SPA, and/or the RNA interference nucleic acid. For example, if the DNA template includes promoters, such as EF1a, or inducible promoters such as the HNF1α-YB TATA promoter, described herein, to drive expression of the CAR or priming receptor, the one or more nucleic acid sequences (e.g., shRNA sequences) can be encoded in the promoter intronic region. In some embodiments, the one or more nucleic acid sequences is encoded in at least one intron region of the nucleic acid insert, module, cassette, or DNA template. In some embodiments, the one or more nucleic acid sequences is encoded in at least one EF1a intron region of the nucleic acid insert, module, cassette, or DNA template.

In some embodiments, the present disclosure contemplates nucleic acid(s), modules, cassettes, or DNA template inserts that comprise one or more transgenes encoding the priming receptors, CARs, SPAs, and/or the RNA interference nucleic acid as described herein. In some embodiments, the DNA template insert or cassette encodes a priming receptor transgene. In some embodiments, the DNA template insert or cassette encodes a chimeric antigen receptor transgene. In some embodiments, the DNA template insert or cassette encodes a synthetic pathway activator transgene. In some embodiments, the DNA template insert or cassette encodes an RNA interference molecule (e.g., a nucleic acid). In some embodiments, the DNA template insert or cassette encodes a first nucleic acid complementary to at least 15 nucleotides of a human FAS mRNA sequence, and a second, third, and/or fourth nucleic acid complementary to at least 15 nucleotides of a human TGBFR2 mRNA sequence. In some embodiments, the DNA template insert or cassette comprises a priming receptor transgene and a chimeric antigen receptor transgene. In some embodiments, the DNA template insert comprises a priming receptor transgene, a chimeric antigen receptor transgene, synthetic pathway activator transgene, a first nucleic acid complementary to at least 15 nucleotides of a human FAS mRNA sequence, and a second, third, and/or fourth nucleic acid complementary to at least 15 nucleotides of a human TGBFR2 mRNA sequence.

In some embodiments, the one or more nucleic acid(s) are encoded on a single DNA template insert or cassette. In some embodiments, the one or more nucleic acid(s) are encoded on multiple DNA template inserts or cassettes. For example, the one or more nucleic acid(s) can be encoded on two, three, or four DNA template inserts.

The DNA template insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7(2): 403-412 (2015)).

The DNA template insert can also comprise a WPRE element. WPRE elements are generally described in Higashimoto, T., et al. Gene Ther 14, 1298-1304 (2007); and Zufferey, R., et al. J Virol. 1999 April; 73(4):2886-92, both of which are hereby incorporated by reference.

The DNA template insert can also comprise an SV40 or a human growth hormone (GH1) polyA tail.

### Cells

Also provided herein are cells or immune cells comprising at least one DNA template non-virally inserted into a target region of the genome of the cell, wherein DNA template encodes the priming receptor and CAR system as described herein. Also provided herein are immune cells comprising the priming receptor that specifically binds Prostate Stem Cell Antigen (PSCA) and the chimeric antigen receptor that specifically binds PSMA.

A cell comprising a DNA template insert at a target locus or safe harbor site as described in the present disclosure can be referred to as an engineered cell. In some embodiments, the cell or immune cell is any cell that can give rise to a pluripotent immune cell. In some embodiments, the immune cell is a primary immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, a hematopoietic cell, a hematopoietic stem cell, an adaptive immune cell, an innate immune cell, a T cell or a T cell progenitor. Non-limiting examples of immune cells that are contemplated in the present disclosure include T cell, B cell, natural killer (NK) cell, NKT/INKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cell is a T cell, NK cells, iPSC, and HSPC. In some embodiments, the engineered cells used in the present disclosure are human cell lines grown in vitro (e.g. deliberately immortalized cell lines, cancer cell lines, etc.).

Also provided herein are populations of cells comprising a plurality of the cells or immune cell. In some embodiments, the genome of at least 20%, 30%, 40%, 50%, 60%, 70%, 80%, 90%, 95%, 99% or greater of the cells comprises the priming receptor and CAR system as described herein.

### Method of Treating Immune-Related Condition of Disease

In another aspect, the disclosure herein provides methods of treating an immune-related condition (e.g., cancer) in an individual comprising administering to the individual an effective amount of a composition comprising a system comprising a priming receptor that specifically binds to PSCA and a chimeric antigen receptor that specifically binds to PSMA, such as a cell composition comprising a system comprising a priming receptor that specifically binds to PSCA and a chimeric antigen receptor that specifically binds to PSMA. In another aspect, the disclosure herein provides methods of enhancing an immune response in an individual comprising administering to the individual an effective amount of a composition comprising a system comprising a priming receptor that specifically binds to PSCA and a chimeric antigen receptor that specifically binds to PSMA, such as a cell composition comprising a system comprising a priming receptor that specifically binds to PSCA and a chimeric antigen receptor that specifically binds to PSMA.

In some embodiments, the target cell is a cell that expresses both PSCA and PSMA. In some embodiments, the target cell is a cell that expresses only PSMA. In some embodiments, the target cell is a cancer cell that expresses both PSCA and PSMA. In some embodiments, the target cell is a cancer cell that expresses only PSMA. In some embodiments, the cancerous or diseased cell expresses both PSCA and PSMA. In some embodiments, the cancerous or diseased cell expresses only PSMA.

In some embodiments, the methods provided herein are useful for the treatment of an immune-related condition in an individual. In one embodiment, the individual is a human. In one embodiment, the immune-related condition is cancer.

In some embodiments, the methods provided herein (such as methods of enhancing an immune response) are useful for the treatment of cancer and as such an individual receiving the system described herein has cancer. In some embodiments, the cancer is a solid tumor. In some embodiments, the cancer is immunoevasive. In some embodiments, the cancer is immunoresponsive. In some embodiments, the cancer is prostate cancer or metastatic Castration-Resistant Prostate Cancer (mCRPC). In some embodiments the prostate cancer is metastatic.

In some embodiments, the treatment results in a decrease in the cancer volume or size. In some embodiments, the treatment results in a decrease in a tumor or metastatic lesion volume or size. In some embodiments, the treatment is effective at reducing a tumor or metastatic lesion volume as compared to the tumor or metastatic lesion volume prior to administration of the antibody. In some embodiments, the treatment results in a decrease in the cancer, tumor or metastatic lesion growth rate. In some embodiments, the treatment is effective at reducing a cancer, tumor or metastatic lesion growth rate as compared to the cancer, tumor or metastatic lesion growth rate prior to administration of the antibody. In some embodiments, the treatment is effective at eliminating a tumor or metastatic lesion.

In some embodiments, PSMA and/or PSCA are expressed at a higher level in the cancer as compared to a non-cancer cell. Levels of PSMA and/or PSCA can be assessed by any technique known in the field, including, but not limited to, protein assays or nucleic assays such as FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

In some aspects, provided herein are methods of inhibiting a target cell in a subject comprising administering the immune cell or population of immune cells disclosed herein to the subject, wherein the immune cell inhibits the target cell. Inhibition of a target cell includes killing of the cell (e.g., the cancer cell), or prevention or reduction in target cell growth, proliferation, or metastasis (e.g., cancer cell growth, proliferation, or metastasis).

### Method of Immune Modulation and Induced Cytolysis

Methods of administration of a cell comprising a system comprising a priming receptor that specifically binds to PSCA and a chimeric antigen receptor that specifically binds to PSMA as described herein can result in modulation of an immune response. Modulation can be an increase or decrease in an immune response. In some embodiments, modulation is an increase in an immune response. In some embodiments, the immune response is secretion of pro-inflammatory cytokines or chemokines, or T cell-mediated cytotoxicity. In some embodiments, the immune response is inducing a cytolytic response (e.g., T cell-mediated cytotoxicity) in a target cell by a cell expressing the system. In some embodiments, the immune response is killing a target cell by a cell expressing the system.

In one aspect, provided herein are methods of inducing cytolysis (e.g., via T cell-mediated cytotoxicity) by or killing a target cell by contacting the target cell with a cell comprising a system comprising a priming receptor that specifically binds to PSCA and a chimeric antigen receptor that specifically binds to PSMA, wherein the target cell expresses PSCA and/or PSMA. In some embodiments, the target cell is a cancer cell. In some embodiments, the contacting happens in vivo in a subject.

In one aspect, administration of a cell comprising a system comprising a priming receptor that specifically binds to PSCA and a chimeric antigen receptor that specifically binds to PSMA as described herein can result in induction of pro-inflammatory molecules, such as cytokines or chemokines. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, M1-like macrophage activation, and NK cell activation. Thus, the administration of a system comprising a priming receptor that specifically binds to PSCA and a chimeric antigen receptor that specifically binds to PSMA can induce multiple anti-tumor immune mechanisms that lead to tumor destruction or cytolysis of tumor cells.

In some embodiments, the target cell is a cell that expresses both PSCA and PSMA. In some embodiments, the disease cell expresses both PSCA and PSMA. In some embodiments, the target cell is a cell that expresses only PSMA. In some embodiments, the disease cell expresses only PSMA.

In one aspect, administration of a cell comprising a synthetic immune receptor that specifically binds to PSCA and/or PSMA or a cell comprising a system comprising a priming receptor that specifically binds to PSCA and a chimeric antigen receptor that specifically binds to PSMA as described herein can result in T cell-mediated cytotoxicity. Generally, cytotoxic T cells kill target cells bearing specific antigen(s), (e.g., such as PSCA and/or PSMA) and do not kill neighboring cells that do not express the specific antigen(s).

In another aspect, provided herein are methods of increasing an immune response (e.g., inducing a pro-inflammatory response or T cell-mediated cytotoxicity) in an individual comprising administering to the individual an effective amount of a cell comprising a system comprising a priming receptor that specifically binds to PSCA and a chimeric antigen receptor that specifically binds to PSMA. In some embodiments, the method of increasing an immune response in a subject comprises administering to the subject a cell comprising a system comprising a priming receptor that specifically binds to PSCA and a chimeric antigen receptor that specifically binds to PSMA.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a composition comprising a system comprising a priming receptor that specifically binds to PSCA and a chimeric antigen receptor that specifically binds to PSMA.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell comprising a system comprising a priming receptor that specifically binds to PSCA and a chimeric antigen receptor that specifically binds to PSMA. In some embodiments, the immune response is enhanced by administration of cell comprising a system comprising a priming receptor that specifically binds to PSCA and a chimeric antigen receptor that specifically binds to PSMA.

In another aspect, the present application provides methods of genetically editing a cell with a system comprising a priming receptor that specifically binds to PSCA and a chimeric antigen receptor that specifically binds to PSMA, which results in the modulation of the immune function of the cell. The modulation can be increasing an immune response. In some embodiments, the modulation is an increase in immune function. In some embodiments, the modulation of function leads to the expression of an PSMA CAR. In some embodiments, the modulation of function leads to the activation of a cell comprising the system.

In some embodiments, the cell is a natural killer (NK) cell, a T cell, a CD8+ T cell, a CD4+ T cell, a primary T cell, or a T cell progenitor.

In some embodiments, the modulation of function of the cells comprising the priming receptor and CAR system as described herein leads to an increase in the cells' abilities to stimulate both native and activated T-cells, for example, by increasing cytokine or chemokine secretion by the cells expressing the priming receptor and CAR system. In some embodiments, the modulation of function enhances or increases the cells' ability to produce cytokines, chemokines, CARs, or costimulatory or activating receptors. In some embodiments, the modulation increases the T-cell stimulatory function of the cells expressing the priming receptor and CAR system, including, for example, the cells' abilities to trigger T-cell receptor (TCR) signaling, T-cell proliferation, or T-cell cytokine production.

In some embodiments, the increased immune response is secretion of cytokines and chemokines. In some embodiments, the priming receptor and CAR system induces increased expression of at least one cytokine or chemokine in a cell as compared to an isotype control cell. In some embodiments, the at least one cytokine or chemokine is selected from the group consisting of: IL-10, IL-2, IL-6, TNFα, GM-CSF, CXCL10, CCL3, CCL4 and IFNγ. In some embodiments, the cytokine or chemokine is IL-10. In some embodiments, the cytokine or chemokine is IL-2. In some embodiments, the cytokine or chemokine is IL-6. In some embodiments, the cytokine or chemokine is TNFα. In some embodiments, the cytokine or chemokine is GM-CSF. In some embodiments, the cytokine or chemokine is CXCL10. In some embodiments, the cytokine or chemokine is CCL3. In some embodiments, the cytokine or chemokine is CCL4. In some embodiments, the cytokine or chemokine is IFNγ. In some embodiments, the cytokine or chemokine secretion is increased a between bout 1-100-fold 1, 5, 10, 20, 30, 40, 50, 60, 70, 80, 90, 100, 1-10, 10-20, 20-30, 30-40, 40-50, 50-60, 60-70, 70-80, 80-90, or 90-100 fold as compared to an untreated cell or a cell treated with an isotype control antibody. In some embodiments, the chemokine is IL-2 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the chemokine is IL-6 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the chemokine is TNFα and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the chemokine is GM-CSF and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the chemokine is CXCL10 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the chemokine is CCL3 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the chemokine is CCL4 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the cytokine is IFNγ and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody.

In some embodiments, the enhanced immune response is anti-tumor immune cell recruitment and activation. In some embodiments, the method further comprises administering an immunotherapy disclosed herein to the subject concurrently with the immune cell. In some embodiments, the method further comprises administering an immunotherapy disclosed herein to the subject subsequently to the immune cell. In some embodiments, the cell or immune cell proliferates in the subject after administration. In some embodiments, the cell or immune cell population expands in the subject after administration.

In some embodiments, the method further comprises determining the number of cells or immune cells disclosed herein in the subject after administration. In some embodiments, the number of cells or immune cells is determined at least or about 5, 6, 7, 8, 9, or 10 days after administration of the cell or immune cell to the subject. In some embodiments, the number of cells or immune cells increases as compared to the number of cells or immune cells administered to the subject. The number of cells or immune cells can be determined by any cell based assays or gene copy assays known to one of skill in the art that assess the expression of the CAR, priming receptor, SPA, and/or shRNA transgenes inserted into the genome of the cells or immune cells disclosed herein. For example, the number of cells or immune cells can be quantified by any cell counting technique known in the field, including, but not limited to image analysis, fluorescence-activated cell sorting (FACS), flow cytometry, Coulter counters, or CASY cell counting technology and combinations thereof. Gene expression can be quantified by any appropriate technique known in the field, including, but not limited to polymerase chain reaction (PCR), quantitative PCR (qPCR), RT-qPCR, microarray, gene array, RNAseq, and combinations thereof.

In some embodiments, the cell expressing the priming receptor and CAR system induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Editing Cells

In some aspects, provide herein are methods of editing a cell, comprising: providing a nuclease domain and a guide RNA, wherein the nucleic acid comprises a nucleic acid disclosed herein, and wherein the 5′ and 3′ ends of the nucleic acid comprise nucleotide sequences that are homologous to genomic sequences flanking an insertion site in the genome of the cell; introducing the nuclease domain and nucleic acid into the cell, wherein the guide RNA specifically hybridizes to a target region of the genome of the cell, and wherein the nuclease domain cleaves the target region to create the insertion site in the genome of the cell; and editing the cell via insertion of the nucleic acid into the insertion site in the genome of the cell.

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (e.g. the adeno-associated virus integration site 1 (AAVS1) safe harbor locus). The DICE (dual integrase cassette exchange) system utilizing phiC31 integrase and Bxb1 integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Also provided herein are methods of making an immune cell comprising introducing a nucleic acid comprising the priming receptor and CAR system as described herein into a primary immune cell. In some embodiments, the immune cell is an isolated cell. In some embodiments, the immune cell is a mammalian cell (e.g., a human cell).

Provided herein are methods of inserting nucleotide sequences greater than about 5 kilobases in length into the genome of a cell, in the absence of a viral vector. In some embodiments, the nucleotide sequence greater than about 5 kilobase in length can be inserted into the genome of a primary immune cell, in the absence of a viral vector

Integration of large nucleic acids, for example nucleic acids greater than 5 kilobase in size, into cells, can be limited by low efficiency of integration, off-target effects and/or loss of cell viability. Described herein are methods and compositions for achieving integration of a nucleotide sequence, for example, a nucleotide sequence greater than about 5 kilobases in size, into the genome of a cell. In some methods the efficiency of integration is increased, off-target effects are reduced and/or loss of cell viability is reduced.

The plasmid can be introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the priming receptor. However, as explained, the design of the transgene cassette ensures that non-virally delivered circuit system receptors do not express CAR until the priming receptor binds to its cognate ligand and releases the cleavable transcription factor.

Initially, a T cell is isolated and optionally activated. The T cell may be obtained from a patient. Thus, the present disclosure provides methods in which immune cells, such as T cells, are harvested from a patient. Then, the plasmid that encodes the CAR and priming receptor are introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The T cells are then expanded and co-cultured to create a sufficient quantity of engineered immune cells to be used as a therapeutic treatment.

Methods for editing the genome of a cell can include a) providing a Cas9 ribonucleoprotein complex (RNP) and a nucleic acid, comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded a nucleic acid, such as a DNA template, wherein the size of the nucleic acid (e.g., DNA template) is greater than about 200, 300, 500, 1000, 1500, 2000, 2500, 3000, 3500, 4000, 4500, or 5000 nucleotides, wherein the 5′ and 3′ ends of the nucleic acid (e.g., DNA template) comprise nucleotide sequences that are homologous to genomic sequences flanking the insertion site, and wherein the molar ratio of RNP to nucleic acid (e.g., DNA template) in the complex is from about 3:1 to about 100:1; and b) introducing the RNP complex and nucleic acid (e.g., DNA template) into the cell.

In some embodiments, the methods described herein provide an efficiency of delivery of the RNP complex and the nucleic acid (e.g., DNA template) of at least about 10%, 15%, 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP complex and the nucleic acid (e.g., DNA template) into the cell. In some cases, the efficiency is determined with respect to the total number of cells (viable or non-viable) in which the RNP complex and the nucleic acid (e.g., DNA template) is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP and nucleic acid (e.g., DNA template) has been introduced. In some cases, the viability of the cells to which the RNP and nucleic acid (e.g., DNA template) has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP and nucleic acid (e.g., DNA template) has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to nucleic acid (e.g., DNA template) can be from about 3:1 to about 100:1. For example, the molar ratio can be from about 3:1 to 10:1, from about 3:1 to about 15:1, 3:1 to about 20:1; 3:1 to about 25:1; from about 3:1 to 50:1, from about 3:1 to 75:1, from about 3:1 to 100:1; from about 5:1 to 10:1, from about 5:1 to about 15:1, 5:1 to about 20:1; 5:1 to about 25:1; from about 5:1 to 50:1, from about 5:1 to 75:1, from about 5:1 to 100:1; from about 8:1 to about 12:1; from about 8:1 to about 15:1, from about 8:1 to about 20:1, from about 8:1 to about 25:1, from about 8:1 to 50:1, from about 8:1 to 75:1, from about 8:1 to 100:1; from about 10:1 to about 15:1, 10:1 to about 20:1, 10:1 to about 25:1; from about 10:1 to 50:1, from about 10:1 to 75:1, or from about 10:1 to 100:1.

In some embodiments, the nucleic acid (e.g., DNA template) is at a concentration of about 2.5 pM to about 25 pM. For example, the concentration of nucleic acid (e.g., DNA template) can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the size or length of the nucleic acid (e.g., DNA template) is greater than about 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10 kb, 11 kb, 12 kb, 13 kb, 14 kb, 15 kb, or 16 kb or any size of nucleic acid (e.g., DNA template) in between these sizes. For example, the size of the DNA template can be about 4.5 kb to about 15 kb, about 4.5 kb to about 14 kb, about 4.5 kb to about 10 kb, about 5 kb to about 15 kb, about 5 kb to about 14 kb, about 5 kb to about 10 kb, about 5 kb to about 9 kb, about 5 kb to about 8 kb, about 5 kb to about 7 kb, about 5 kb to about 6 kb, about kb 6 to about 15 kb, about kb 6 to about 14 kb, about kb 6 to about 10 kb, about 6 kb to about 9 kb, about 6 kb to about 8 kb, about 6 kb to about 7 kb, about 7 kb to about 15 kb, about 7 kb to about 14 kb, about 7 kb to about 10 kb, about 7 kb to about 9 kb, about 7 kb to about 8 kb, about 8 kb to about 15 kb, about 8 kb to about 14 kb, about 8 kb to about 10 kb, about 8 kb to about 9 kb, about 9 kb to about 15 kb, about 9 kb to about 14 kb, about 9 kb to about 13 kb, about 9 kb to about 12 kb, about 9 kb to about 11 kb, about 9 kb to about 10 kb, about 10 kb to about 15 kb, about 10 kb to about 14 kb, about 10 kb to about 13 kb, about 10 kb to about 12 kb, or about 10 kb to about 11 kb.

In some embodiments, the amount of nucleic acid (e.g., DNA template) is about 1 μg to about 10 μg. For example, the amount of nucleic acid (e.g., DNA template) can be about 1 μg to about 2 μg, about 1 μg to about 3 μg, about 1 μg to about 4 μg, about 1 μg to about 5 ug, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of nucleic acid (e.g., DNA template) is about 3 μg to about 4 μg, about 3 μg to about 5 μg, about 3 μg to about 6 ug, about 3 μg to about 7 μg, about 3 μg to about 8 μg, about 3 μg to about 9 μg, or about 3 μg to about 10 μg. In some embodiments, the amount of nucleic acid (e.g., DNA template) is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of nucleic acid (e.g., DNA template) is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some cases, the size of the nucleic acid (e.g., DNA template) is large enough and in sufficient quantity to be lethal as naked DNA. In some embodiments, the DNA template encodes a heterologous protein or a fragment thereof. In some embodiments, the nucleic acid (e.g., DNA template) encodes at least one gene. In some embodiments, the DNA template encodes at least two genes. In some embodiments, the nucleic acid (e.g., DNA template) encodes one, two, three, four, five, six, seven, eight, nine, ten, or more genes.

In some embodiments, the nucleic acid (e.g., DNA template) includes regulatory sequences, for example, a promoter sequence and/or an enhancer sequence to regulate expression of the heterologous protein or fragment thereof after insertion into the genome of a cell. In some embodiments, the promoter is an inducible promoter. In some embodiments, the inducible promoter comprises one or more HNF1α enhancer elements. In some embodiments, the inducible promoter comprises a YB-TATA promoter element. In some embodiments, the inducible promoter comprises a sequence as set forth in SEQ ID NO: 7. In some embodiments, the promoter is a constitutive promoter. In some embodiments, the constitutive promoter is an EF1α promoter. In some embodiments, the constitutive promoter comprises the sequence of SEQ ID NO: 15.

In some cases, the nucleic acid (e.g., DNA template) is a linear DNA template. In some cases, the nucleic acid (e.g., DNA template) is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, an RNP and nucleic acid (e.g., DNA template) complex is formed by incubating the RNP with the nucleic acid (e.g., DNA template) for less than about one minute to about thirty minutes, at a temperature of about 20 °C to about 25 °C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20 °C, 21 °C, 22 °C, 23 °C, 24 °C, or 25 °C. In another example, the RNP can be incubated with the nucleic acid (e.g., DNA template) for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20 °C to about 25 °C. In some embodiments, the RNP-DNA template complex and the cell are mixed prior to introducing the RNP-DNA template complex into the cell. In some embodiments, the RNP and nucleic acid (e.g., DNA template) and the cell are mixed prior to introducing the RNP and nucleic acid (e.g., DNA template) into the cell.

In some embodiments introducing the RNP complex and nucleic acid (e.g., DNA template) comprises electroporation. Methods, compositions, and devices for electroporating cells to introduce a RNP and nucleic acid (e.g., DNA template) can include those described in the examples herein. Additional or alternative methods, compositions, and devices for electroporating cells to introduce a RNP and nucleic acid (e.g., DNA template) can include those described in WO/2006/001614 or Kim, J. A. et al. Biosens. Bioelectron. 23, 1353-1360 (2008). Additional or alternative methods, compositions, and devices for electroporating cells to introduce a RNP and nucleic acid (e.g., DNA template) can include those described in U.S. Patent Appl. Pub. Nos. 2006/0094095; 2005/0064596; or 2006/0087522. Additional or alternative methods, compositions, and devices for electroporating cells to introduce an RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP and nucleic acid (e.g., DNA template) can include those described in Geng, T. et al., J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a nucleic acid (e.g., DNA template), a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP and nucleic acids (e.g., DNA templates) comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a nucleic acid (e.g., DNA template) at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4-CD8-T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP and nucleic acid (e.g., DNA template) is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of engineered (e.g., recombinant) T cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

### Insertion Sites

Methods for editing the genome of a T cell, specifically, include a method of editing the genome of a human T cell comprise inserting a nucleic acid sequence or construct into a target region in exon 1 of the TCR-α subunit (TRAC) gene in the human T cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of a T cell also include a method of editing the genome of a human T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of a T cell, specifically, include a method of editing the genome of a human T cell comprise inserting a nucleic acid sequence or construct into a target region of a genomic safe harbor (GSH).

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

Examples of safe harbor sites are provided in International Patent Application WO2022093846A1, published on May 5, 2022, hereby incorporated by reference in its entirety. In some embodiments, the safe harbor site is selected from Table 4 of WO2022093846A1. In some embodiments, the safe harbor site is the GS94 safe harbor site. In some embodiments, the GS94 integration site is a safe harbor site at chr11:128340000-128350000. In some embodiments, the GS94 integration site is at chr11:128342576. In some embodiments, the GS94 integration site is targeted by the sgRNA (sgRNA_94) provided in SEQ ID NO: 5. In some embodiments, the safe harbor site is the GS102 safe harbor site. In some embodiments, the GS102 integration site is a safe harbor site at chr15:92830000-92840000. In some embodiments, the GS102 integration site is at chr15:92831131. In some embodiments, the GS102 integration site is targeted by the sgRNA (sgRNA_102) comprising the sequence ATACACGCACACTTGCAGAA (SEQ ID NO: 64).

Additional safe harbor sites are provided in Sadelain, M., et al. (2012). Safe harbours for the integration of new DNA in the human genome. Nature reviews Cancer, 12(1), 51-58; Irion et al. 2007 (“Identification and targeting of the ROSA26 locus in human embryonic stem cells.” Nature biotechnology 25.12 (2007): 1477-1482; and Pellenz 3 et al. “New Human Chromosomal Sites with “Safe Harbor” Potential for Targeted Transgene Insertion.” Human gene therapy vol. 30, 7 (2019): 814-828; the relevant disclosures of which are herein incorporated by reference in their entirety.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kB, about 125 kB, about 150 kB, about 175 kB, about 200 kB, about 225 kB, about 250 kB, about 275 kB, about 300 kB, about 325 kB, about 350 kB, about 375 kB, about 400 kB, about 425 kB, about 450 kB, about 475 kB, about 500 kB, about 525 kB, about 550 kB away from the safe harbor locus (integration site).

In some embodiments, the present disclosure contemplates inserts that comprise one or more transgenes. The transgene can encode a therapeutic protein, an antibody, a peptide, or any other gene of interest. The transgene integration can result in, for example, enhanced therapeutic properties. These enhanced therapeutic properties, as used herein, refer to an enhanced therapeutic property of a cell when compared to a typical immune cell of the same normal cell type. For example, a T cell having “enhanced therapeutic properties” has an enhanced, improved, and/or increased treatment outcome when compared to a typical, unmodified and/or naturally occurring T cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site. In some embodiments, the insert size comprises at least about 4.5 kilobasepairs (kb) to about 10 kilobasepairs (kb). In some embodiments, the insert size comprises about 5000 nucleotides or more basepairs. In some embodiments, the insert size comprises up to 4.5, 4.8, 5, 6, 7, 8, 9, 10, 11, 12, 13, 14, 15, 16, 17, 18, 19, 20 kbp (kilo basepairs) or the sizes in between. In some embodiments, the insert size is greater than 4.5, 4.8, 5, 6, 7, 8, 9, 10, 11, 12, 13, 14, 15, 16, 17, 18, 19, 20 kbp or the sizes in between. In some embodiments, the insert size is within the range of 4.5-15 kbp or is any number in that range. In some embodiments, the insert size is within the range of 4.8-8.3 kbp or is any number in that range. In some embodiments, the insert size is within the range of 5-8.3 kbp or is any number in that range. In some embodiments, the insert size is within the range of 5-15 kbp or is any number in that range. In some embodiments, the insert size is within the range of 4.5-20 kbp or is any number in that range. In some embodiments, the insert size is 5-10 kbp. In some embodiments, the insert size is 4.5-10, 5-10, 6-10, 7-10, 8-10, 9-10 kbp. In some embodiments, the insert size is 4.5-11, 6-11, 7-11, 8-11, 9-11, or 10-11 kbp. In some embodiments, the insert size is 4.5-12, 6-12, 7-12, 8-12, 9-12, 10-12, or 11-12 kbp. In some embodiments, the insert size is 4.5-13, 6-13, 7-13, 8-13, 9-13, 10-13, 11-13, or 12-13 kbp. In some embodiments, the insert size is 4.5-14, 6-14, 7-14, 8-14, 9-14, 10-14, 11-14, 12-14 or 13-14 kbp. In some embodiments, the insert size is 4.5-15, 6-15, 7-15, 8-15, 9-15, 10-15, 11-15, 12-15, 13-15, or 14-15 kbp. In some embodiments, the insert size is 4.5-16, 6-16, 7-16, 8-16, 9-16, 10-16, 11-16, 12-16, 13-16, 14-16 or 15-16 kbp. In some embodiments, the insert size is 4.5-17, 6-17, 7-17, 8-17, 9-17, 10-17, 11-17, 12-17, 13-17, or 14-17, 15-17 or 16-17 kbp. In some embodiments, the insert size is 4.5-18, 6-18, 7-18, 8-18, 9-18, 10-18, 11-18, 12-18, 13-18, 14-18, 15-18, 16-18 or 17-18 kbp. In some embodiments, the insert size is 4.5-19, 6-19, 7-19, 8-19, 9-19, 10-19, 11-19, 12-19, 13-19, 14-19, 15-19, 16-19, 17-19, or 18-19 kbp. In some embodiments, the insert size is 4.5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprise a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), priming receptors, CARs, mAbs, RNAi molecules, etc. In some embodiments, the insert is a human sequence. In some embodiments, the insert is chimeric. In some embodiments, the insert is a multi-gene/multi-module therapeutic cassette. A multi-gene/multi-module therapeutic cassette refers to an insert or cassette having one or more than one receptor (e.g., synthetic receptors such as a CAR or a priming receptor), other exogenous protein coding sequences, non-coding RNAs, transcriptional regulatory elements, and/or insulator sequences, etc.

In some embodiments, the nucleic acid sequence is inserted into the genome of the cell such as an immune cell or T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. Pat. Nos. 11,033,584B2 and 11,814,624B2, the relevant disclosures of which are herein incorporated by reference in their entirety.

### CRISPR-Cas Editing

One effective example of gene editing is the CRISPR-Cas approach (e.g. CRISPR-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a nuclease such as a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540(7631), 144-149, the relevant disclosures of which are herein incorporated in their entirety).

Methods for conducing gene editing using CRISPR-Cas approaches are known to those of ordinary skill in the art. (See, for example, U.S. Ser. Nos. 16/312,676, 15/303,722, and 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount. The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of engineered (e.g., recombinant) cell lines expressing a protein of interest.

The engineered cells of the present disclosure can be any cell, generally a mammalian cell, generally a human cell that has been modified by integrating a transgene at a safe harbor locus described herein. Exemplary cells are provided in the Cells section.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective number of the immune cells comprising the system may be administered for the treatment of cancer. The appropriate dosage of immune cells comprising the system may be determined based on the type of cancer to be treated, the type of the immune cell comprising the system, the severity and course of the cancer, the clinical condition of the individual, the individual's clinical history and response to the treatment, and the discretion of the attending physician.

Determining Expression of PSCA and/or PSMA

Also provided herein are methods of treating a cancer in a subject in need thereof comprising: determining or having determined the expression of PSCA in the subject; and/or determining or having determined the expression of PSMA in the subject; and administering or having administered to the subject a system comprising a priming receptor comprising a first extracellular antigen-binding domain that specifically binds to PSCA, a chimeric antigen receptor comprising a second extracellular antigen-binding domain that specifically binds to PSMA.

Also provided herein are methods of treating a disease in a subject comprising: determining or having determined the presence of PSCA-positive (PSCA+) cells from a cancer sample obtained from the subject and determining or having determined the presence of PSMA-positive (PSMA+) cells from a cancer sample obtained from the subject; and administering the cell or immune cell as disclosed herein or the pharmaceutical composition as disclosed herein to the subject.

Also provided herein are methods of treating a disease in a subject comprising: determining or having determined the presence of PSCA-positive (PSCA+) cells from a cancer sample obtained from the subject and administering the cell or immune cell as disclosed herein or the pharmaceutical composition as disclosed herein to the subject.

Also provided herein are methods of treating a disease in a subject comprising: determining or having determined the presence of PSMA-positive (PSMA+) cells from a cancer sample obtained from the subject; and administering the cell or immune cell as disclosed herein or the pharmaceutical composition as disclosed herein to the subject.

Also provided herein are methods of treating a cancer in a subject in need thereof comprising: determining or having determined the expression of PSCA in the subject; and/or determining or having determined the expression of PSMA in the subject; and administering or having administered to the subject a cell comprising a priming receptor comprising a first extracellular antigen-binding domain that specifically binds to PSCA, a chimeric antigen receptor comprising a second extracellular antigen-binding domain that specifically binds to PSMA, and optionally a synthetic pathway activator inserted into a target region of the genome of the cell. In some embodiments, the cell is an immune cell. In some embodiments, the immune cell is a primary immune cell.

In some embodiments, the method further comprises determining or having determined the expression level of PSCA and/or PSMA in a biological sample from the individual. In some embodiments the biological sample includes, but is not limited to a body fluid, a tissue sample, an organ sample, urine, feces, blood, saliva, CSF and any combination thereof. In some embodiments the biological sample is derived from a tumor tissue. In some embodiments, the expression level of PSCA and/or PSMA comprises the mRNA expression level of PSCA and/or PSMA. In some embodiments, the expression level of PSCA and/or PSMA comprises the protein expression level of PSCA and/or PSMA. In some embodiments the expression level of PSCA and/or PSMA is detected in the sample using a method selected from the group consisting of FACS, Western blot, ELISA, immunoprecipitation, immunohistochemistry, monoplex immunohistochemistry, multiplex immunohistochemistry, immunofluorescence, radioimmunoassay, dot blotting, immunodetection methods, HPLC, surface plasmon resonance, optical spectroscopy, mass spectrometry, qPCR, RT-qPCR, multiplex qPCR or RT-qPCR, RNA-seq, microarray analysis, SAGE, MassARRAY technique, Luminex, MSD, and FISH, and combinations thereof.

In some aspects, provided herein are methods of determining an expression level of PSCA and/or PSMA protein in a sample from a subject comprising contacting the sample with an anti-PSCA and/or PSMA antibody and performing an immunohistochemistry assay. In some embodiments, the anti-PSCA antibody comprises the PSCA antibody as set forth in SEQ ID NOs: 44-52. In some embodiments, the anti-PSMA antibody comprises the PSMA antibody as set forth in SEQ ID NOs: 32-40.

In another aspect, the present invention provides methods for identifying an individual who may respond to immunotherapy (e.g. with the PSCA and/or PSMA protein system or a cell comprising a PSCA and/or PSMA protein system described herein) for the treatment of an immune-related condition (e.g. cancer) comprising: detecting the expression level of PSCA in a biological sample from the individual; detecting the expression level of PSMA in a biological sample from the individual; and determining based on the expression level of PSCA and/or PSMA, whether the individual may respond immunotherapy, wherein co-expression of PSCA and PSMA in a tumor sample from the individual indicates that the individual may respond to immunotherapy. In some embodiments, the PSCA and/or PSMA expression in the individual has already been determined. In some embodiments, the expression level of PSCA and/or PSMA comprises the mRNA expression level of PSCA and/or PSMA. In other embodiments, the expression level of PSCA and/or PSMA comprises the protein expression level of PSCA and/or PSMA.

In some embodiments the expression level of PSCA and/or PSMA is detected in the sample using a nucleic acid or protein assay. Exemplary a nucleic acid or protein assays include, but are not limited to, FACS, Western blot, ELISA, immunoprecipitation, immunohistochemistry, monoplex immunohistochemistry, multiplex immunohistochemistry, immunofluorescence, radioimmunoassay, dot blotting, immunodetection methods, HPLC, surface plasmon resonance, optical spectroscopy, mass spectrometry, qPCR, RT-qPCR, multiplex qPCR or RT-qPCR, RNA-seq, microarray analysis, SAGE, MassARRAY technique, Luminex, MSD, and FISH, and combinations thereof. In these embodiments, the anti-PSCA and/or PSMA antibody binds to the PSCA and/or PSMA protein, but does not necessarily have to affect a biological response, such as ADCC. In some embodiments the biological sample is derived from a tumor tissue. In some embodiments the biological sample includes, but is not limited to a body fluid, a tissue sample, an organ sample, urine, feces, blood, saliva, CSF and any combination thereof.

In some embodiments, the assay is an immunohistochemistry assay and the anti-PSCA antibody comprises the antibody as set forth in SEQ ID NOs: 44-52. In some embodiments, the assay is an immunohistochemistry assay and the anti-PSMA antibody comprises the antibody as set forth in SEQ ID NOs: 32-40.

### Pharmaceutical Compositions

The engineered (e.g., recombinant) cells provided herein can be administered as part of a pharmaceutical compositions. These compositions can comprise, in addition to one or more of the engineered (e.g., recombinant) cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the nucleic acid, system, or cell compositions described herein along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the therapeutic compositions described herein (e.g., any one or more of the antibodies, receptors, priming receptors, CARs, SPAs, nucleic acids, or cells described herein), with one or more pharmaceutically acceptable excipients.

The present application also provides articles of manufacture comprising any one of the therapeutic compositions or kits described herein (e.g., any one or more of the antibodies, receptors, priming receptors, CARs, SPAs, nucleic acids, or cells described herein). Examples of an article of manufacture include vials (including sealed vials) and bags.

### ADDITIONAL EMBODIMENTS

Embodiment 1. One or more nucleic acid(s), wherein the one or more nucleic acids encode at least one of:
a. a first antibody or antigen-binding domain that specifically binds to Prostate Stem Cell Antigen (PSCA), comprising a first variable heavy (VH) chain sequence comprising three heavy chain CDR sequences, CDR-H1, CDR-H2, and CDR-H3, of the VH sequence set forth in SEQ ID NO: 45, and a variable light (VL) chain sequence comprising three light chain CDR sequences, CDR-L1, CDR-L2, and CDR-L3, of the VL sequence set forth in SEQ ID NO: 46; and/or
b. a second antibody or antigen-binding domain that specifically binds Prostate-Specific Membrane Antigen (PSMA), comprising a second variable heavy (VH) chain sequence comprising three heavy chain CDR sequences, CDR-H1, CDR-H2, and CDR-H3, of the VH sequence set forth in SEQ ID NO: 33, and a variable light (VL) chain sequence comprising three light chain CDR sequences, CDR-L1, CDR-L2, and CDR-L3, of the VL sequence set forth in SEQ ID NO: 34.

Embodiment 2. The one or more nucleic acid(s) of embodiment 1, wherein:
a. the first variable heavy (VH) chain sequence comprises a CDR-H1 comprising the sequence set forth in SEQ ID NO: 47, a CDR-H2 comprising the sequence set forth in SEQ ID NO: 48, a CDR-H3 comprising the sequence set forth in SEQ ID NO: 49, a CDR-L1 comprising the sequence set forth in SEQ ID NO: 50, a CDR-L2 comprising the sequence set forth in SEQ ID NO: 51, and a CDR-L3 comprising the sequence set forth in SEQ ID NO: 52; and/or
b. the second variable heavy (VH) chain sequence comprises a CDR-H1 comprising the sequence set forth in SEQ ID NO: 35, a CDR-H2 comprising the sequence set forth in SEQ ID NO: 36, a CDR-H3 comprising the sequence set forth in SEQ ID NO: 37, a CDR-L1 comprising the sequence set forth in SEQ ID NO: 38, a CDR-L2 comprising the sequence set forth in SEQ ID NO: 39, and a CDR-L3 comprising the sequence set forth in SEQ ID NO: 40.

Embodiment 3. The one or more nucleic acid(s) of embodiment 1, wherein the first VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 45 and/or the first VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 46.

Embodiment 4. The nucleic acid(s) of embodiment 1, wherein the first antibody or antigen-binding domain comprises an scFv, optionally wherein the scFv comprises a sequence at least 80%, 85%, 90%, 91%, 92%, 93%, 94%, 95%, 96%, 97%, 98%, 99%, or 100% identical to the sequence as set forth in SEQ ID NO: 44.

Embodiment 5. The one or more nucleic acid(s) of embodiment 1, wherein the second VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 33 and/or the second VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 34.

Embodiment 6. The one or more nucleic acid(s) of embodiment 1, wherein the second antibody or antigen-binding domain comprises an scFv, optionally wherein the scFv comprises a sequence at least 80%, 85%, 90%, 91%, 92%, 93%, 94%, 95%, 96%, 97%, 98%, 99%, or 100% identical to the sequence as set forth in SEQ ID NO: 32.

Embodiment 7. One or more nucleic acid(s), wherein the one or more nucleic acids encode a system comprising:
a. a first chimeric polypeptide comprising a priming receptor comprising a first extracellular antigen-binding domain that specifically binds Prostate Stem Cell Antigen (PSCA), wherein the first extracellular antigen-binding domain comprises a first variable heavy (VH) chain sequence comprising three heavy chain CDR sequences, CDR-H1, CDR-H2, and CDR-H3, of the VH sequences set forth in SEQ ID NO: 45, and a light chain comprising a first variable light (VL) chain sequence comprising three light chain CDR sequences, CDR-L1, CDR-L2, and CDR-L3, of the VL sequences set forth in SEQ ID NO: 46, optionally wherein:
   i. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52; and
b. a second chimeric polypeptide comprising a chimeric antigen receptor (CAR) comprising a second extracellular antigen-binding domain that specifically binds to Prostate-Specific Membrane Antigen (PSMA).

Embodiment 8. The nucleic acid(s) of embodiment 7, wherein the first VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 45 and/or the first VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 46.

Embodiment 9. The nucleic acid(s) of embodiment 7, wherein the first extracellular antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 44.

Embodiment 10. The nucleic acid(s) of embodiment 7, wherein the second extracellular antigen-binding domain comprises a heavy chain comprising a second variable heavy (VH) chain sequence comprising three heavy chain CDR sequences, CDR-H1, CDR-H2, and CDR-H3, of the VH sequences set forth in SEQ ID NO: 33, and a light chain comprising a second variable light (VL) chain sequence comprising three light chain CDR sequences, CDR-L1, CDR-L2, and CDR-L3, of the VL sequence set forth in SEQ ID NO: 34, optionally wherein
a. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

Embodiment 11. The nucleic acid(s) of embodiment 7, wherein the second VH comprises a sequence at least 80%, 85%, 90%, 91%, 92%, 93%, 94%, 95%, 96%, 97%, 98%, 99%, or 100% identical to the sequence as set forth in SEQ ID NO: 33 and/or the second VL comprises a sequence at least 80%, 85%, 90%, 91%, 92%, 93%, 94%, 95%, 96%, 97%, 98%, 99%, or 100% identical to the sequence as set forth in SEQ ID NO: 34.

Embodiment 12. The nucleic acid(s) of embodiment 7, wherein the second extracellular domain comprises a sequence at least 80%, 85%, 90%, 91%, 92%, 93%, 94%, 95%, 96%, 97%, 98%, 99%, or 100% identical to the sequence as set forth in SEQ ID NO: 32. Embodiment 13. The nucleic acid(s) embodiment 7, wherein
a. the priming receptor comprises, from N-terminus to C-terminus,
   i. the first antigen-binding domain;
   ii. a first transmembrane domain comprising one or more ligand-inducible proteolytic cleavage sites; and
   iii. an intracellular domain comprising a human or humanized transcriptional effector, wherein binding of the first antigen-binding domain to human PSCA results in cleavage at the one or more ligand-inducible proteolytic cleavage sites; and/or
b. the CAR comprises, from N-terminus to C-terminus,
   i. a second antigen-binding domain;
   ii. a second transmembrane domain;
   iii. an intracellular co-stimulatory domain; and
   iv. an intracellular activation domain.

Embodiment 14. The nucleic acid(s) embodiment 7, wherein the priming receptor comprises a sequence at least 80%, 85%, 90%, 91%, 92%, 93%, 94%, 95%, 96%, 97%, 98%, 99%, or 100% identical to the sequence as set forth in SEQ ID NO: 54 and/or the CAR comprises a sequence at least 80%, 85%, 90%, 91%, 92%, 93%, 94%, 95%, 96%, 97%, 98%, 99%, or 100% identical to the sequence as set forth in SEQ ID NO: 42.

Embodiment 15. The nucleic acid(s) of embodiment 7, further comprising
a. an optional third chimeric polypeptide comprising a synthetic pathway activator (SPA); and
b. at least one nucleic acid sequence complementary to at least 15 nucleotides of each of:
   i. a nucleic acid encoding human Fas Cell Surface Death Receptor (FAS) comprising the sequence set forth in SEQ ID NO: 3; and
   ii. a nucleic acid encoding human Transforming Growth factor (TGF)-β Receptor 2 (TGFBR2) comprising the sequence set forth in SEQ ID NO: 4.

Embodiment 16. One or more nucleic acid(s), wherein the one or more nucleic acids encode a system comprising:
a. a first chimeric polypeptide comprising a priming receptor comprising a first extracellular antigen-binding domain that specifically binds Prostate Stem Cell Antigen (PSCA); and
b. a second chimeric polypeptide comprising a chimeric antigen receptor (CAR) comprising a second extracellular antigen-binding domain that specifically binds to Prostate-Specific Membrane Antigen (PSMA), wherein the second extracellular antigen-binding domain comprises a heavy chain comprising a second variable heavy (VH) chain sequence comprising three heavy chain CDR sequences, CDR-H1, CDR-H2, and CDR-H3, of the VH sequences set forth in SEQ ID NO: 33, and a light chain comprising a second variable light (VL) chain sequence comprising three light chain CDR sequences, CDR-L1, CDR-L2, and CDR-L3, of the VL sequence set forth in SEQ ID NO: 34, optionally wherein
   i. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

Embodiment 17. One or more expression vector(s) comprising the one or more nucleic acid(s) of embodiment 1.

Embodiment 18. One or more expression vector(s) comprising the one or more nucleic acid(s) of embodiment 7.

Embodiment 19. An antibody or antigen-binding domain encoded by the one or more nucleic acid(s) of embodiment 1.

Embodiment 20. A system encoded by the one or more nucleic acids of embodiment 7.

Embodiment 21. A cell or population of cells comprising the one or more nucleic acid(s) of embodiment 1.

Embodiment 22. A cell or population of cells comprising the one or more nucleic acid(s) of embodiment 7.

Embodiment 23. A pharmaceutical composition comprising the nucleic acid(s) of embodiment 1, and a pharmaceutically acceptable excipient.

Embodiment 24. A pharmaceutical composition comprising the cell or population of cells of embodiment 21, and a pharmaceutically acceptable excipient.

Embodiment 25. A pharmaceutical composition comprising the cell or population of cells of embodiment 22, and a pharmaceutically acceptable excipient.

Embodiment 26. A method of editing a cell, comprising inserting the nucleic acid of embodiment 1 into the cell.

Embodiment 27. A method of editing a cell, comprising inserting the nucleic acid of embodiment 7 into the cell.

Embodiment 28. A method of modulating the activity of a cell or immune cell comprising:
a. obtaining a cell or immune cell comprising one or more nucleic acids of embodiment 1; and
b. contacting the cell or immune cell with a cell expressing PSCA, wherein binding of the priming receptor to PSCA on the cell induces activation of the priming receptor and expression of the chimeric antigen receptor and wherein binding of the chimeric antigen receptor to PSMA on a target cell expressing PSMA modulates the activity of the immune cell.

Embodiment 29. A method of treating a disease or increasing an immune response in a subject comprising administering to the subject a cell comprising the one or more nucleic acids of embodiment 1.

Embodiment 30. A method of treating a disease or increasing an immune response in a subject comprising administering to the subject a cell comprising the one or more nucleic acids encoding the system of embodiment 7.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: In Vitro Synthesis and Characterization of PSCA and PSMA Logic Gates

### Materials and Methods

### PSCA Antigen Binding Protein Generation and Selection

PSCA antigen binding proteins (e.g., antibody or antigen binding fragment thereof) were generated following an immunization and screening campaign. Briefly, PSCA antibodies were discovered from a humanized transgenic mouse immunization campaign with recombinant C-terminally His-tagged PSCA protein, which resulted in identification of 38 unique antibody sequences. All PSCA scFv antigen binding proteins were evaluated in a priming receptor screen where T cells expressing the PSCA antigen binding proteins in a priming receptor (primeR) architecture were assessed in vitro in an exemplary integrated circuit (IC) for basal CAR expression, CAR induction and cytotoxicity (data not shown). The top 5 PSCA primeRs were selected for integrated circuit (IC, or logic gate) screening.

### PSMA Antigen Binding Protein Generation and Selection

PSMA antigen binding proteins (e.g., antibody or antigen binding fragment thereof) were generated following an immunization and screening campaign. Briefly, PSMA antibodies were discovered from a humanized transgenic mouse immunization campaign with recombinant N-terminally His-tagged PSMA protein which resulted in 184 unique antibody sequences. A subset of 50 scFv antigen binding proteins were selected for use as a CAR based on expression in an exemplary integrated circuit (IC). The top 10 PSMA antigen binding proteins were then selected for additional integrated circuit (IC) screening based on basal CAR expression and cytotoxicity (data not shown).

### Logic Gate Screening

From these screens, a total of 165 logic gated circuits comprising receptors containing PSCA and PSMA antigen binding proteins were generated and tested in a screen. Assays used for assessing circuits included short and long-term cytotoxicity experiments, as well as CAR induction basal expression phenotype (data not shown).

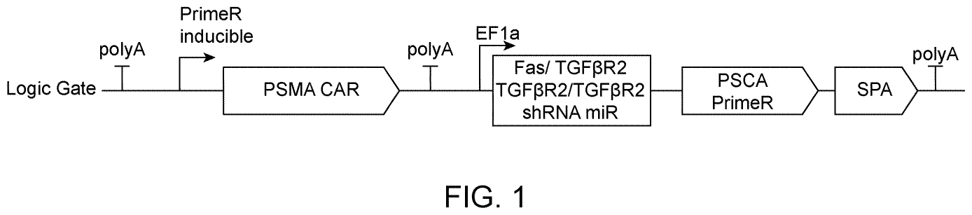

A diagram of an exemplary logic gate circuit (also called an integrated circuit or “IC”) transgene cassette is provided in FIG. 1. The exemplary IC construct comprises a constitutively expressed PSCA priming receptor, an inducible PSMA CAR, a constitutively expressed shRNA cassette targeting FAS and TGFBR2, and a synthetic pathway activator (SPA).

Selected circuits from the above were further optimized resulting in the generation of 418 additional circuits for characterization. Cytotoxicity results were used to narrow down to 20 top candidates. Additional assays including CAR induction, CAR off kinetics, and cytokine secretion were utilized to select the top five circuits, termed Logic Gate 1 (LG 1), Logic Gate 2 (LG 2), Logic Gate 3 (LG 3), Logic Gate 4 (LG 4), and Logic Gate 5 (LG 5) (data not shown).

Table 1 provides the nucleotide positions of the elements included in the expression vector for Logic Gate 1 (LG 1). LG 1 comprises a PSCA priming receptor (SEQ ID NO: 54) comprising a VH shown in SEQ ID NO: 45 and a VL as shown in SEQ ID NO: 46, a PSMA CAR (SEQ ID NO: 42) comprising a VH shown in SEQ ID NO: 33 and a VL as shown in SEQ ID NO: 34, a quad Fas/3×TGFBR2 shRNA cassette as shown in SEQ ID NO: 20 and a synthetic pathway activator (SPA) as shown in SEQ ID NO: 56. The full transgene cassette comprising the selected priming receptor and CAR with the shRNA cassette and SPA is provided in SEQ ID NO: 58. Additionally, a T cell comprising LG 1 but without the inclusion of the SPA component was generated for downstream head to head analysis.

### Integrated Circuit Construct Expression in T Cells (ICT Cells)

Integrated circuit T (ICT) cells were generated through site directed CRISPR mediated knock in (KI). Primary human T cells were activated for two days using CD3-CD28 beads. At day 2, beads were removed followed by the delivery of the ICT transgene to the GS94 site in the genome of the T cells. Transgene integration was performed using a CRISPR-based process and electroporation step that combined activated T cells, CRISPR/Cas9 RNP targeting the GS94 non-coding autosomal integration site, and plasmid DNA constituting a repair template to effect insertion of the transgene cassette via cellular DNA repair machinery.

The GS94 CRISPR/Cas9 RNP used was generated by complexing single guide RNA (sgRNA, SEQ ID NO: 5) with recombinant Streptococcus pyogenes Cas9 (SpCas9). The sgRNA contained a protospacer sequence directing the CRISPR/Cas9 RNP to the GS94-transgene integration site. The plasmid DNA repair template contained the ICT transgene cassette, flanked by 450 base pair (bp) sequences homologous to the regions flanking the integration site to effect repair-mediated insertion.

Following electroporation, cells were recovered and expanded in T cell media for 7 days. When indicated, negative control T cells were generated using a mock electroporation process that edited T cells with ribonucleoprotein (RNP) in the absence of donor plasmid and are referred to as “RNP control.”

ICT cells were assessed for transgene KI and the expression of the priming receptor and CAR using flow based staining. ICT cells at day 7 post activation were stained with anti-Whitlow linker and anti-G4S linker antibodies for 30 min at 4 °C. Following activation, ICT cells were washed in FACs buffer and analyzed by flow cytometry for priming receptor and CAR expression.

### ICT Cell Quantification and Basal CAR Expression

T cells expressing the ICs (ICTs or ICT cells) were generated as described above from the T cells of 2 donors. On day 9 post activation, ICTs were measured for CAR and priming receptor expression by Whitlow (GSTSGSGKPGSGEGSTKG (SEQ ID NO: 66)) and G4S linker (SEQ ID NO: 65) staining. % KI was quantified by summing the % of T cells in a sample that were priming receptor positive (PrimeR+) or chimeric antigen positive (CAR+). Before co-culture setup, ICTs were normalized to the same % KI using the addition of donor matched RNP only cell. Basal CAR expression was measured during assay set up by measuring the fraction of KI+ cells that were CAR+.

CAR on/Off Kinetics

The kinetics of the Logic Gate 1-5 (LG 1-5) cells were assessed by induction of the circuits on plates coated with or without recombinant PSCA protein. Surface expression levels of primeR and CAR were measured by flow cytometry after 96 hours. The induced cells were then removed from antigen exposure and loss of CAR surface expression was monitored by flow every 1-2 days for a week.

### Mixed Co-Culture Cytotoxicity

ICT cells expressing Logic Gates 1-5 (LG 1-5) were co-cultured with PSMA+/PSCA− and PSMA+/PSCA+ luciferase expressing cells at 1:3 E:T ratio for 72 hours at 37 °C. In this assay, PC3 cells containing knockout of endogenous PSMA and PSCA were transformed to express PSCA and/or PSMA recombinantly at tumor-matched levels. Target PSCA cell percentage was varied based on a mixture of heterogeneous cell populations. Following incubation, cytotoxicity was measured using a luciferase reporter assay. Data are presented as the mean±standard error mean of 3 donors. ICT-mediated PSCA+ target cell killing was normalized to 100% PSCA, except for the RNP-electroporated negative control using a luciferase reporter assay.

### Cytotoxicity in 786-O-PSCA Cells

ICT cells expressing LG 1-5 ICs were co-cultured with 786-O-PSCA cells (786-O-TKO (triple knockout of beta-2 microglobulin)-PSCA-H cells engineered to express PSCA) at varying E: T ratios for 72 hours at 37 °C. Following incubation, cytotoxicity was measured using a luciferase reporter assay. Data are presented as the mean±standard error mean of 3 donors.

### LG 1 Sensitivity Cytotoxicity

LG 1 ICT cells were co-cultured with cell lines expressing below average PSMA and PSCA levels at a 3:1 E:T ratio for 72 hours at 37 °C. Data are presented as the mean±standard error mean of 2 donors.

### Cytokine Secretion

To further assess the specificity and function of ICT cells expressing Logic Gate 1 with and without SPAs, supernatants were collected from target PC3 tumor cells co-cultured with the ICT cells in a cytotoxicity assay (Effector: Target ratio of 1:1, two 48 hour co-culture stimulations). Following incubation, supernatants were collected at endpoint and cytokine release levels were measured using a Luminex assay. Data from 2 donors are shown.

### Results

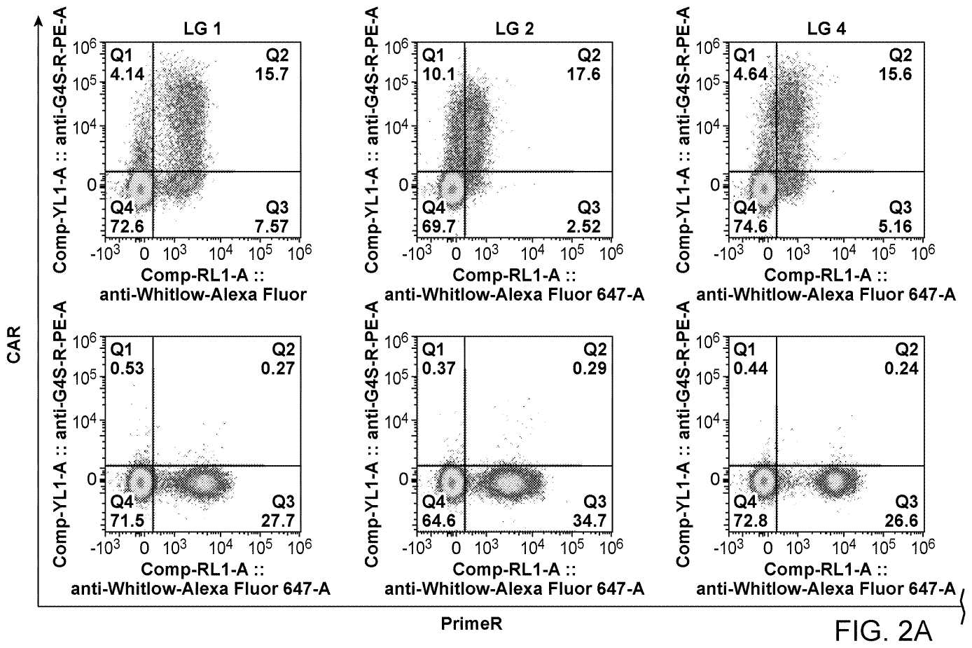

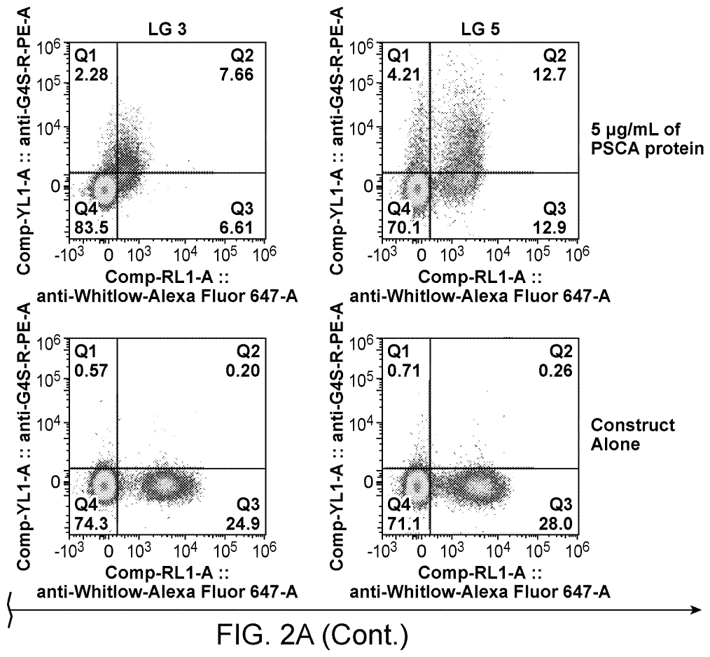

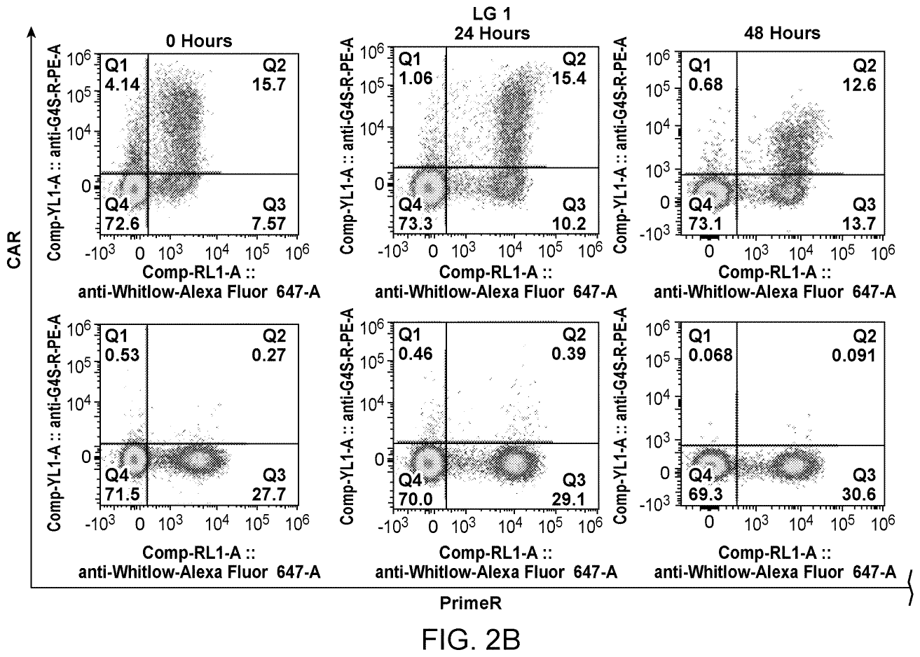

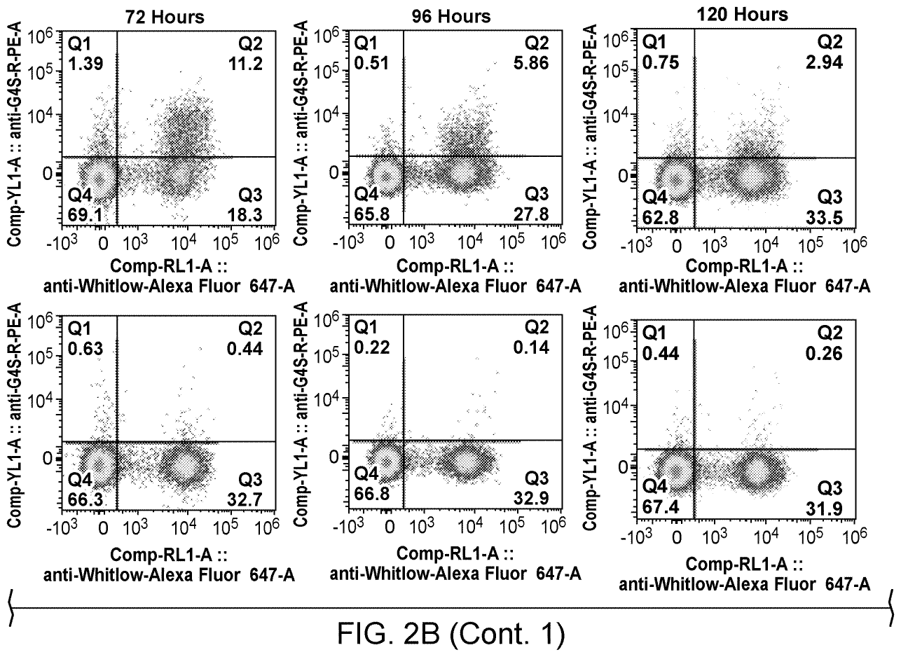

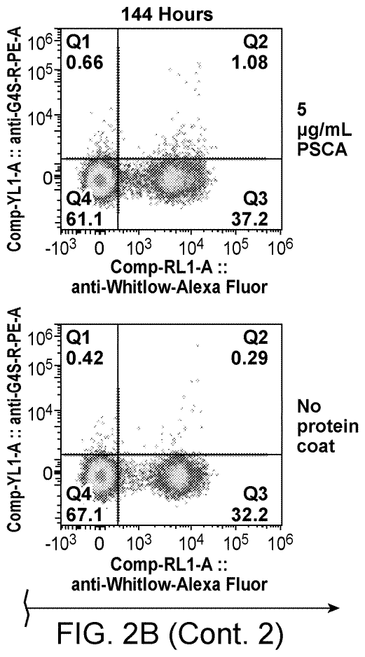

The top five LG (LG 1-5) T cell candidates were characterized using in vitro experiments. CAR induction upon incubation with prime antigen for all 5 LG ICs was assessed and all LG 1-5 T cells induced CAR expression after prime antigen incubation (FIG. 2A). Flow plots of CAR expression in LG 1 T cells over time following removal of prime-antigen are provided in FIG. 2B, showing that CAR expression decreased after removal of the priming antigen. Thus, LG 1 demonstrated robust CAR expression in response to appropriate stimuli. LG 1 also demonstrated a unique profile where high levels of CAR were induced while still maintaining priming receptor (primeR) expression (FIGS. 2A and 2B).

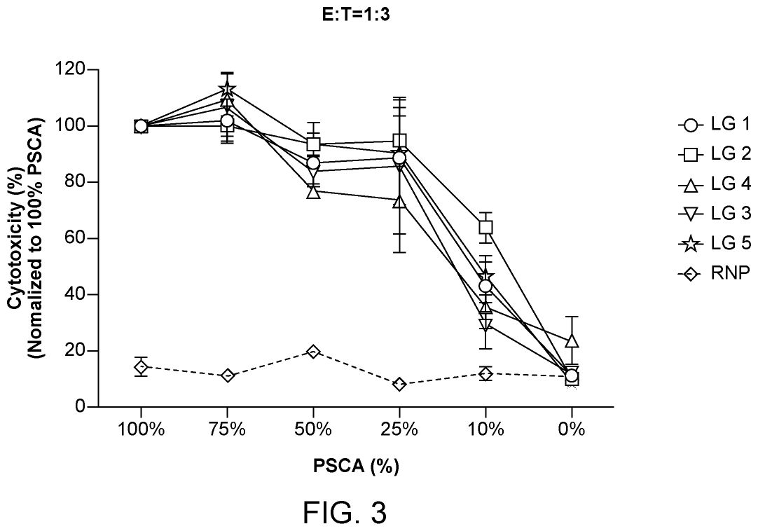

Characterization of LG ICs in a mixed co-culture assay showed all LG candidates had activity even when only 10% of tumor cells expressed PSCA (FIG. 3), indicating a strong cytotoxic profile with minimal prime antigen stimulation.

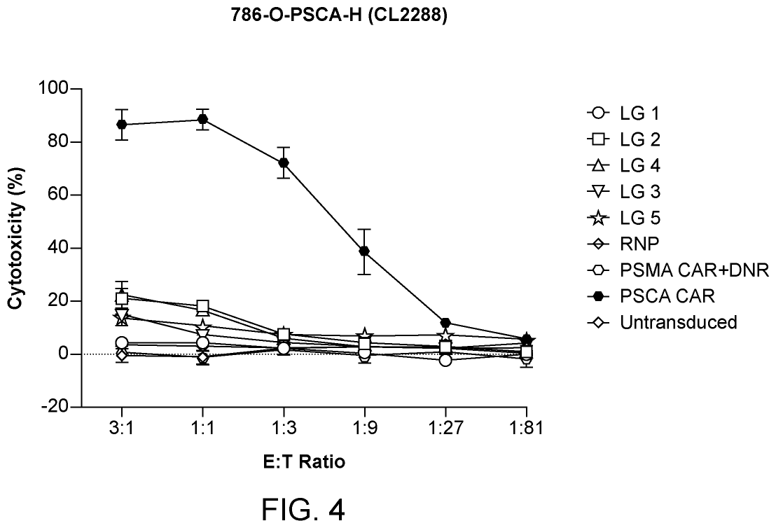

To assess prime-dependent activity, cytotoxicity was measured after incubation of the T cells with PSCA-only expressing cells (786-O-PSCA-H) at various E: T ratios (FIG. 4). Minimal cytotoxic activity was observed across all constructs, with LG 1 exhibiting the least prime antigen-only induced activity.

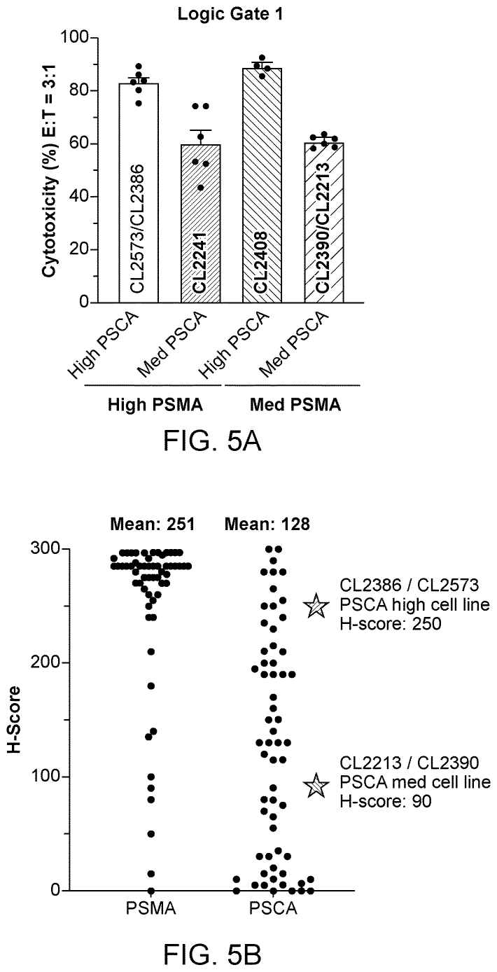

The sensitivity of LG 1 to cells expressing PSCA below the average patient expression was also assessed using cell lines that exhibit below-average expression levels of PSMA and PSCA (medium PSCA and/or medium PSMA). FIG. 5B shows the expression levels of PSMA or PSCA in the cell lines used for the cytotoxicity sensitivity analysis. As shown in FIG. 5A, LG 1 retained killing activity against cells with medium PSCA and medium PSMA expression levels.

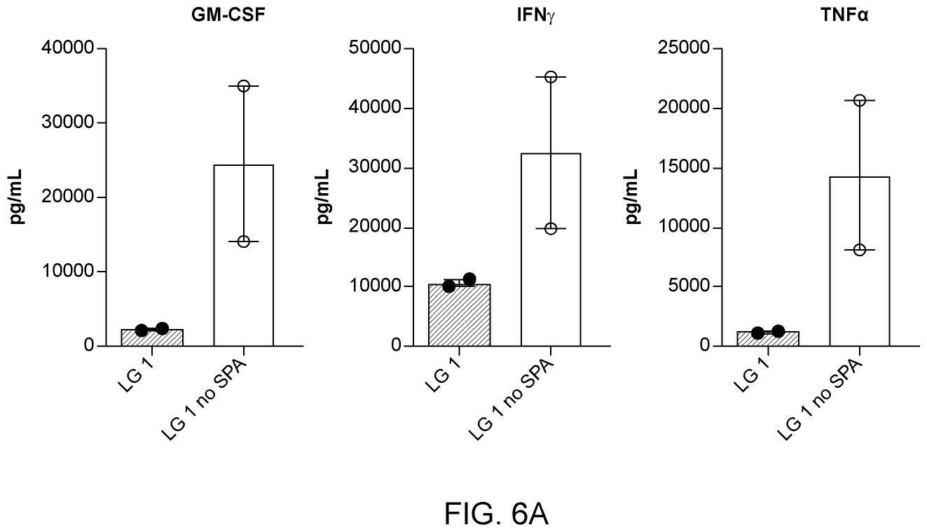

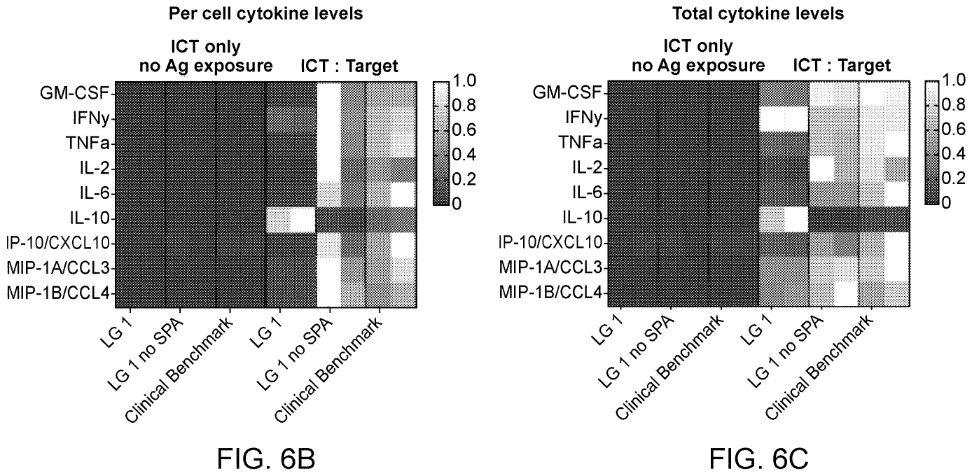

Cytokine secretion in T cells expressing LG 1 with and without expression of the SPA was also assessed after incubation with target PC3 tumor cells at a 1:1 ratio. FIG. 6A shows that T cells with LG 1 produced less pro-inflammatory cytokines associated with hyper-immune syndromes such as cytokine release syndrome (CRS) compared to T cells lacking the SPA portion of LG 1. Additionally, FIGS. 6B and 6C show the per cell and absolute concentrations for most pro-inflammatory cytokines. Lower expression of most pro-inflammatory cytokines (GM-CSF, IFNγ, TNFα, IL-2, IL-6, CXCL10, CCL3 and CCL4) was observed in the T cells expressing the LG 1 comprising the SPA as compared to T cells expressing LG 1 without the SPA. Higher expression of IL-10 was observed in the T cells expressing the LG 1 comprising the SPA as compared to T cells expressing LG 1 without the SPA. This reduction in inflammatory cytokines and increase in IL-10 expression per cell was unexpected.

### Example 2: In Vivo Efficacy of PSMA and PSCA Logic Gate T Cells

### Materials and Methods

### Dual Flank Model

Human 786-O-TKO cells were engineered to express either PSCA and PSMA or PSMA only. 786-O-PSMA+ and 786-O-PSCA+/PSMA+ cells were inoculated into the left and right dorsal flank respectively of six-eight weeks old, female NSG MHC I/II DKO mice. Day 45 post tumor inoculation, mean tumor volume of 150-200 mm³ was reached on each flank and tumor-bearing animals were randomized into various treatment groups such that mean tumor volume per group on the right flank was within 10% of the overall mean. Seven mice/group were injected intravenously with a single dose of 1×10⁶ of PrimeR+ ICT cells expressing one of LG 1-5, a PSMA constitutive CAR with a dominant negative TGFβ receptor (PSMA CAR+DNR), RNP or PBS control. Tumor volumes and body weight were recorded bi-weekly. Tumor volume was calculated as per formula ½*L*W2, where L is tumor length and W is tumor width. Data represents a single donor study with 7 mice per group, mean and SEM plotted.

### Single Flank PSMA Only Model (Prime Antigen-Independent Killing Assay)

Human 786-O-TKO cells were engineered to express PSMA only. 786-O-PSMA+ cells were inoculated into six-eight weeks old, female NSG MHC I/II DKO mice. Day 45 post tumor inoculation, mean tumor volume of 150 mm³ was reached and tumor-bearing animals were randomized into various treatment groups such that mean tumor volume per group was within 10% of the overall mean. Seven mice/group were injected intravenously with a single dose of 0.3×10⁶ or 1×10⁶ of PrimeR+ICT cells expressing LG 1-5, PSMA constitutive CAR with a dominant negative TGFβ receptor (PSMA CAR+DNR), RNP or PBS control. Tumor volumes and body weight were recorded bi-weekly. Tumor volume was calculated as per formula ½*L*W2, where L is tumor length and W is tumor width. Data represents a single donor study with 7 mice per group, mean and SEM plotted.

### Single Flank PSCA Only Model (Prime Antigen-Only Killing Assay)

Human 786-O-TKO cells were engineered to express PSCA only. 786-O-PSCA+/PSMA− cells were inoculated into six-eight weeks old, female NSG MHC I/II DKO mice. Day 45 post tumor inoculation, mean tumor volume of 150 mm³ was reached and tumor-bearing animals were randomized into various treatment groups such that mean tumor volume per group was within 10% of the overall mean. Seven mice/group were injected intravenously with a single dose of 1×10⁶ of PrimeR+ ICT cells expressing LG 1-5, PSCA constitutive CAR (PSMA CAR), RNP or PBS control. Tumor volumes and body weight were recorded bi-weekly. Tumor volume was calculated as per formula ½*L*W2, where L is tumor length and W is tumor width. Data represents a single donor study with 7 mice per group, mean and SEM plotted.

### Single Flank PC3 Efficacy Model

Human PC3-TKO (triple knockout of beta-2 microglobulin, PSCA, and PSMA) cells were engineered to express PSCA and PSMA. PC3-PSCA+/PSMA+ cells were inoculated into six-eight weeks old, male NSG MHC I/II DKO mice. Day 23 post tumor inoculation, mean tumor volume of 150 mm³ was reached and tumor-bearing animals were randomized into various treatment groups such that mean tumor volume per group was within 10% of the overall mean. Eight mice/group were injected intravenously with a single dose of 0.75×10⁵ or 0.3×10⁶ of PrimeR+ICT cells, PSMA targeting chimeric antigen receptor (CAR) with a dominant negative TGFb receptor (PSMA CAR+DNR), RNP or PBS control. Tumor volumes and body weight were recorded bi-weekly. Tumor volume was calculated as per formula ½*L*W2, where L is tumor length and W is tumor width. Data represents a single donor study with 8 mice per group, mean and SEM plotted.

### Results

### Dual Flank Model

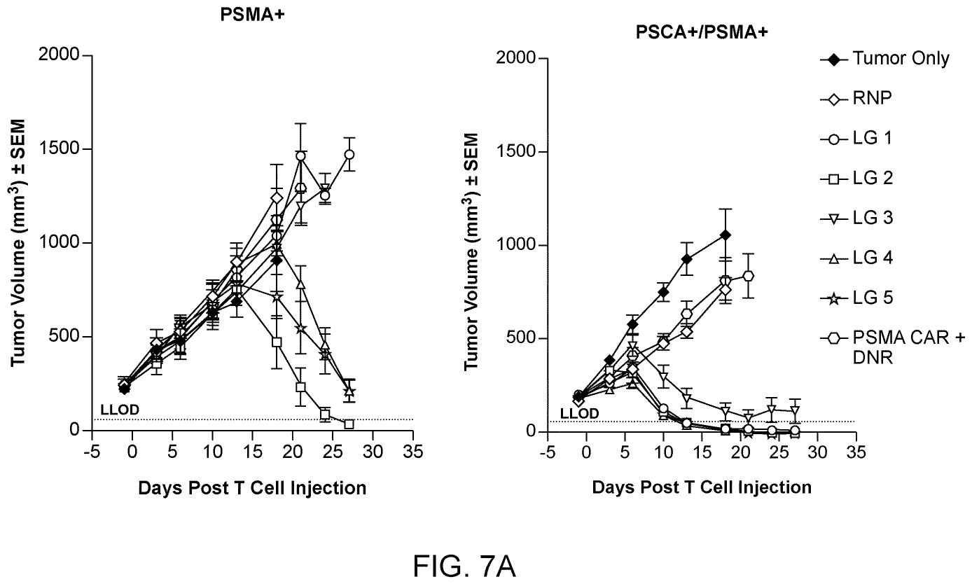

The ICTs expressing LG 1-5 ICs showed on-target, on-tumor and on-target, off-tumor specificity in a dual flank model for two donors. FIGS. 7A and 7B show the tumor volume post-tumor implant in mice treated with ICTs expressing Logic Gates 1-5, a PSMA targeting chimeric antigen receptor (CAR) with a dominant negative TGFb receptor (CAR+DNR), RNP (ribonucleoprotein/Cas protein only) as a negative control or PBS generated from T cells from either donor 1 (FIG. 7A) or donor 2 (FIG. 7B). Greater tumor growth inhibition (TGI) was observed for LG 1 and LG 3 ICs in the dual positive PSMA+/PSCA+ flank (FIG. 7A and FIG. 7B, right panel) than the single positive PSMA-only flank (FIG. 7A and FIG. 7B, left panel). Thus, the dual flank xenograft model shows that logic gate 1 (LG 1) and logic gate 3 (LG 3) ICT cells more selectively killed tumors that express both PSCA and PSMA, and not tumors that express PSMA alone.

### Single Flank PSMA Only Model (Prime Antigen-Independent Killing Assay)

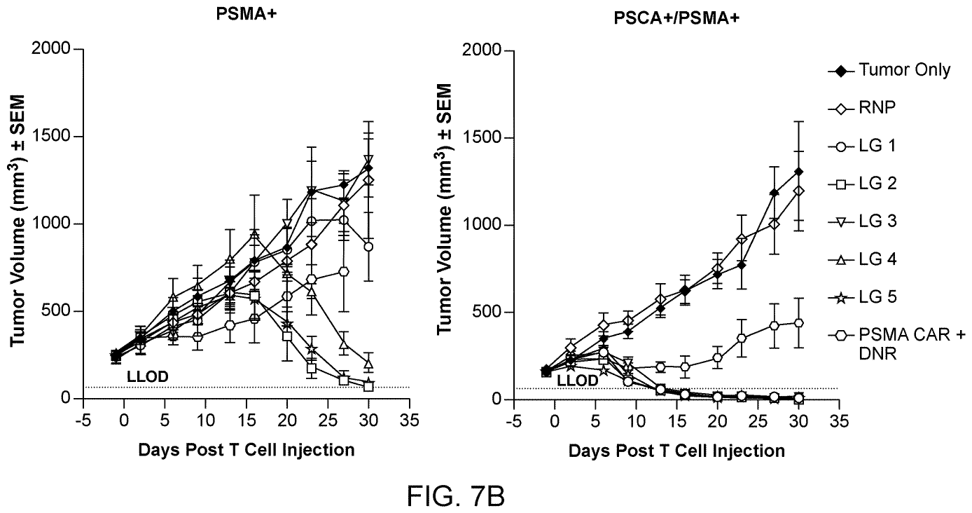

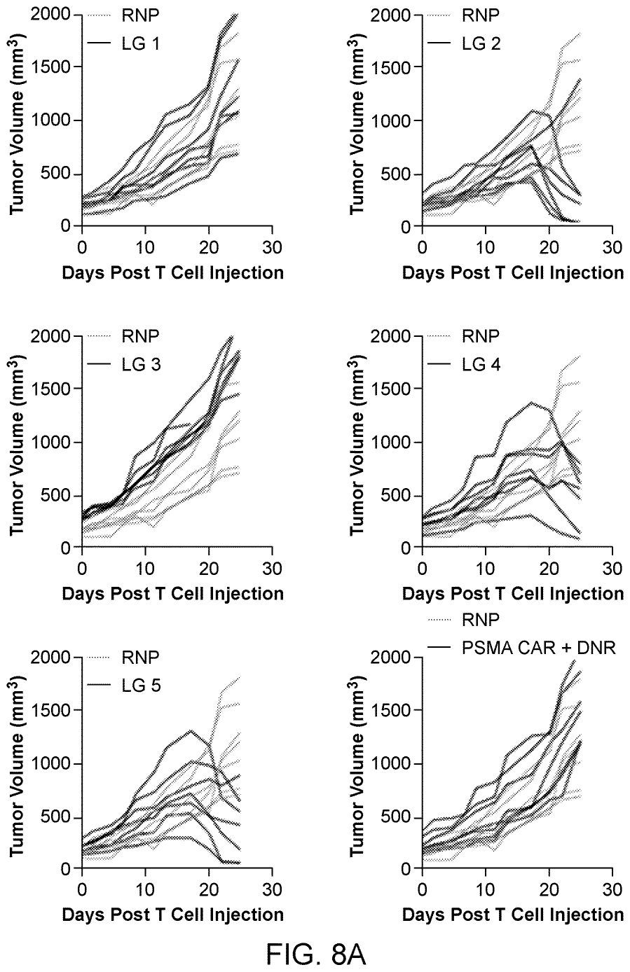

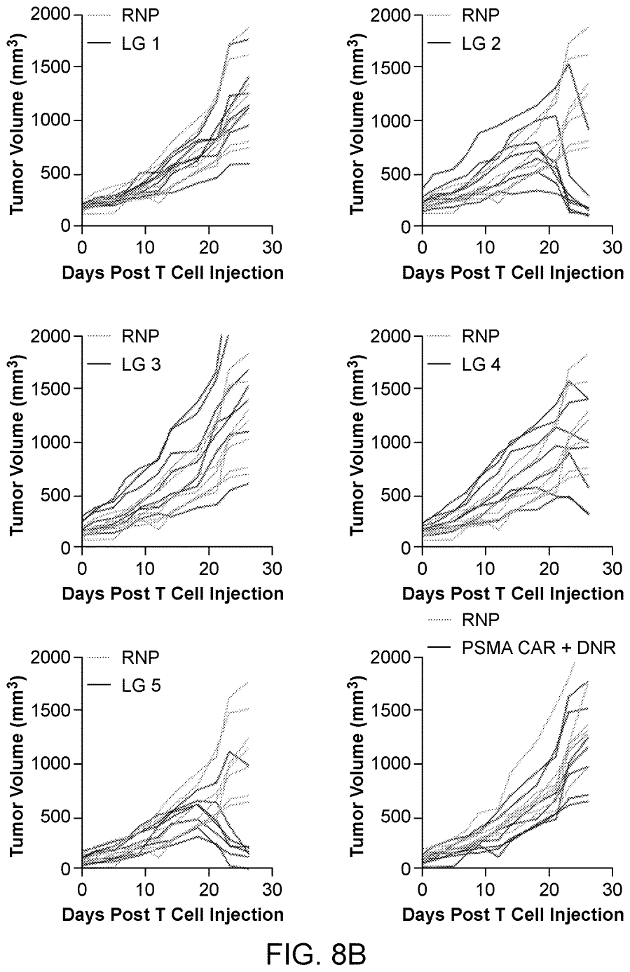

To assess PSCA prime antigen independent killing (e.g., killing of cells expressing the PSMA cytolytic target antigen without the initial PSCA priming to induce CAR expression), a single-flank model with PSMA only expressing tumor cells was used. FIGS. 8A and 8B show the tumor volume in mice treated with T cells expressing Logic Gates 1-5, an exemplary PSMA CAR+DNR, or RNP. LG 1 and LG 3 T cells showed no significant prime antigen-independent killing of tumor cells in mice treated with either the 1×10⁶ (FIG. 8A) and 0.3×10⁶ (FIG. 8B) doses. Thus, the PSMA single flank model demonstrated that T cells expressing LG 1 and LG 3 demonstrated high selectivity with no killing after contacting cells expressing only the PSMA cytolytic antigen.

### Single Flank PSCA Only Model (Prime Antigen-Only Killing Assay)

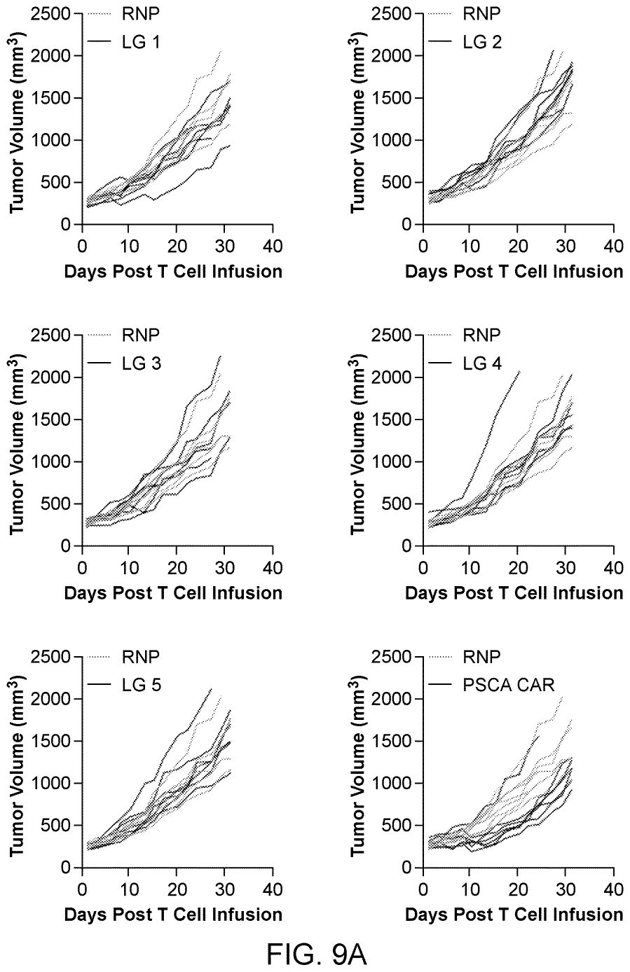

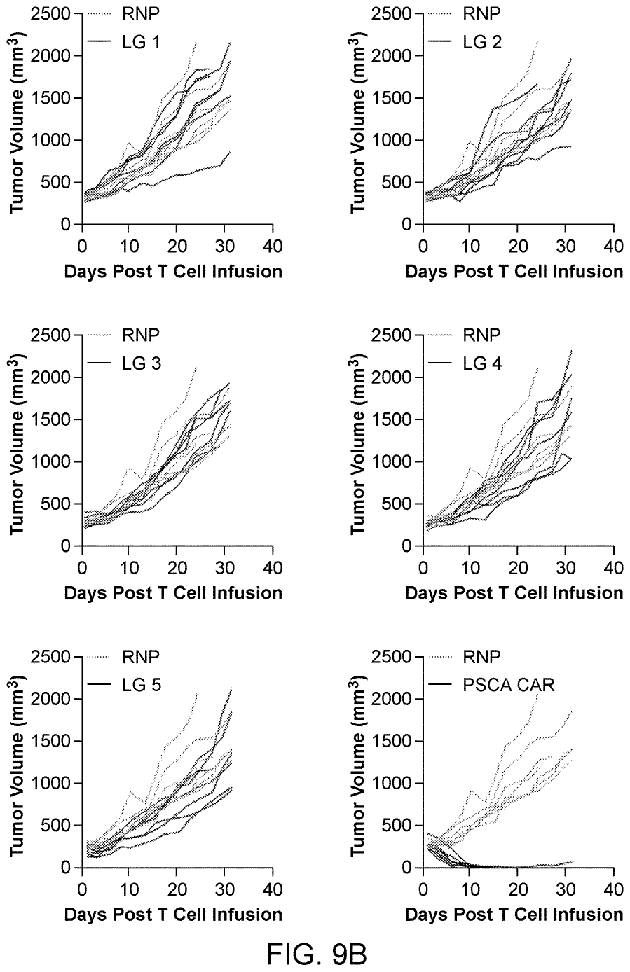

To assess killing of PSCA prime antigen only expressing cells (e.g., off target killing of cells expressing only the PSCA priming antigen), a single-flank model with PSCA only expressing tumor cells was used. FIGS. 9A and 9B show the tumor volume in mice treated with T cells expressing Logic Gates 1-5, an exemplary PSCA targeting chimeric antigen receptor (PSCA CAR) as a positive control, or RNP as a negative control. None of LG 1-5 T cells exhibited prime antigen-only killing at either the 1×10⁶ (FIG. 9A) or 0.3×10⁶ (FIG. 9B) doses. The positive control PSCA CAR did result in killing of tumor cells. Thus, none of the LG ICs demonstrated cytolytic activity in vivo in the absence of CAR tumor target.

Taken with the single flank PSMA prime antigen independent killing data, this data indicates that LG 1 and LG 3 T cells showed the highest specificity and selectivity for tumors that express both PSCA and PSMA, and not tumors that express PSCA or PSMA alone.

### Single Flank PC3-PSCA+/PSMA+ Efficacy Model

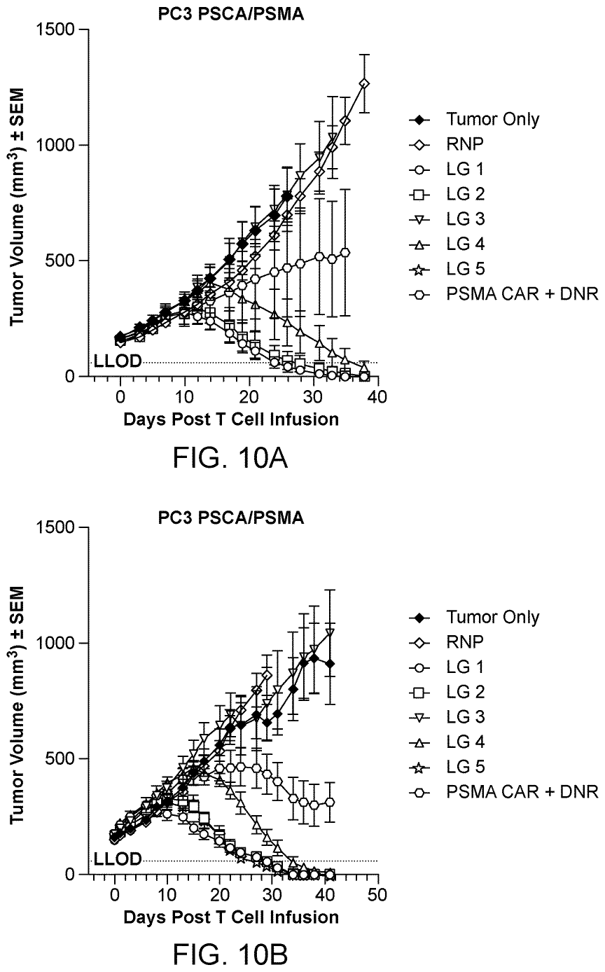

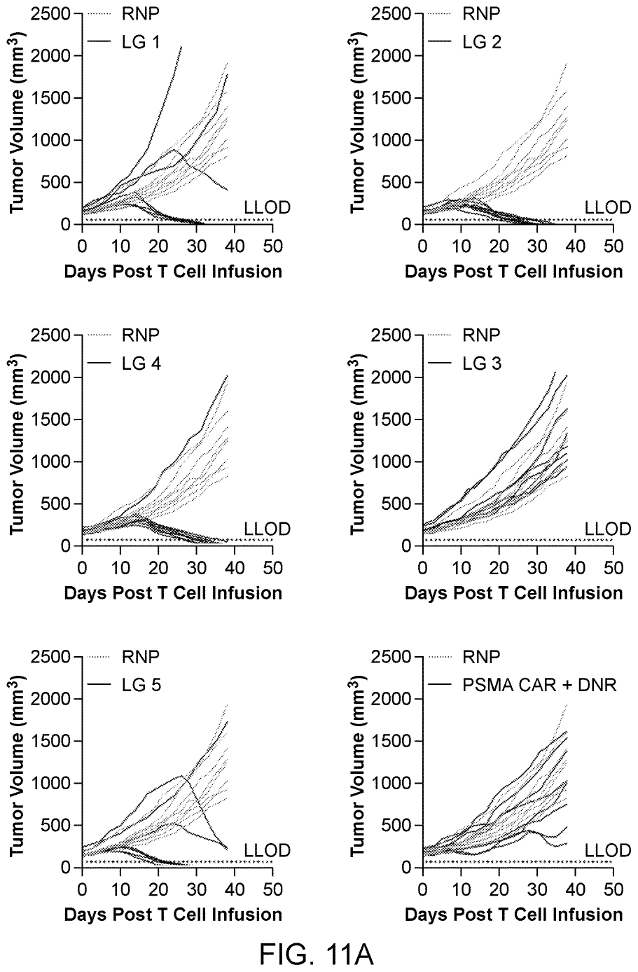

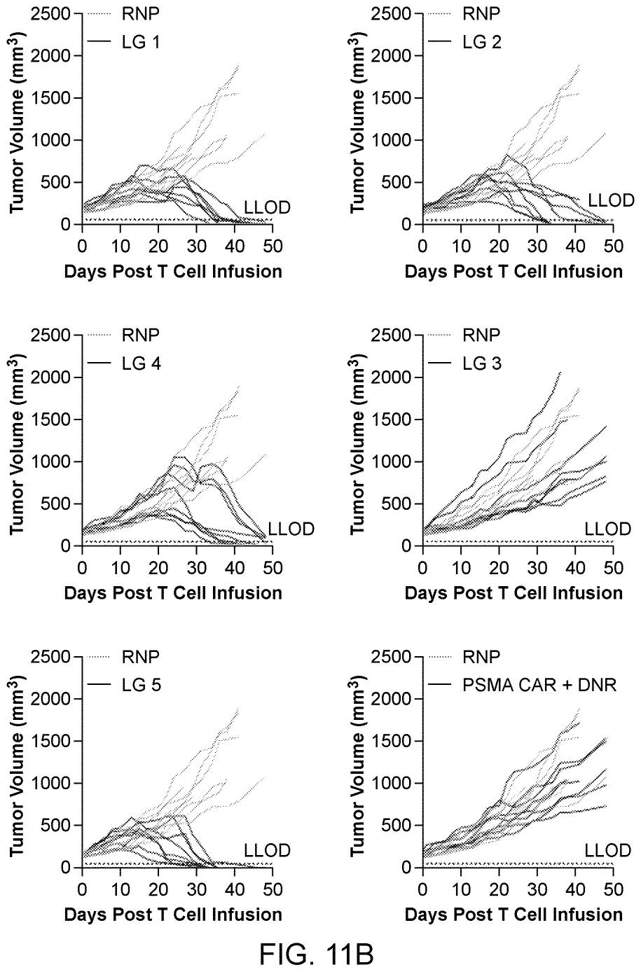

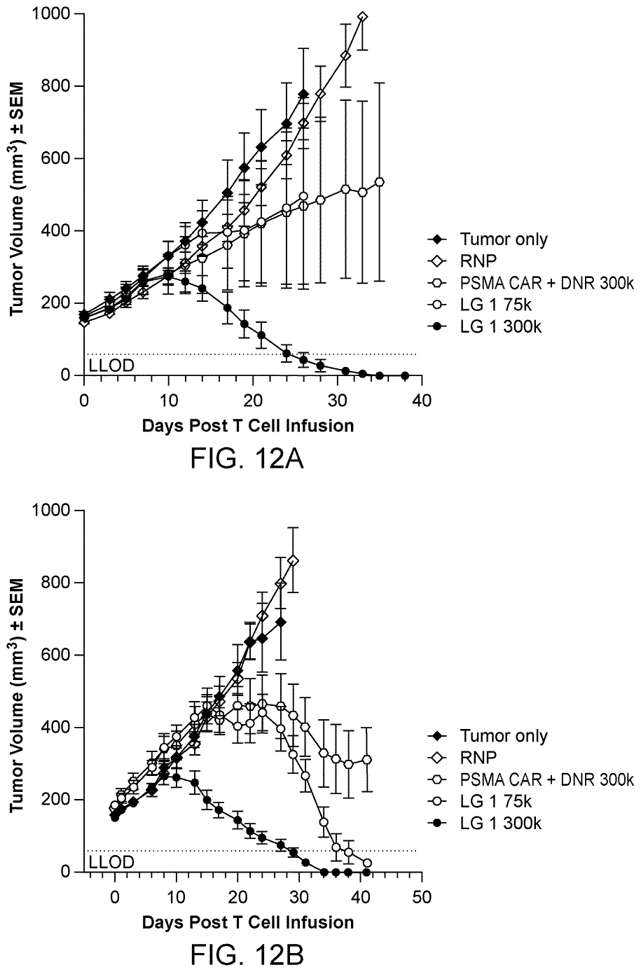

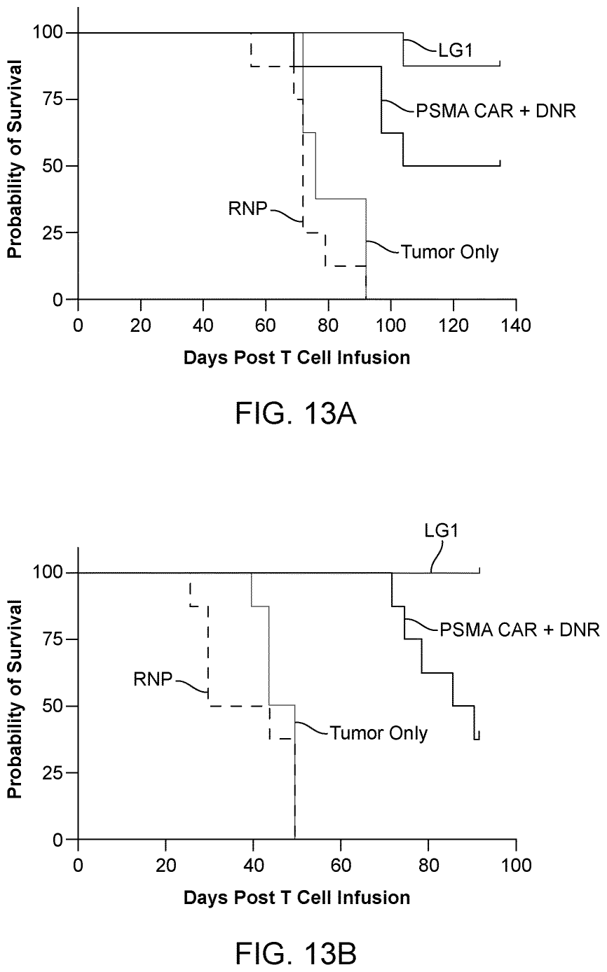

A PC3 prostate cancer cell in vivo model with cells expressing both PSCA and PSMA was used to rank the potency of the LG 1-5 T cells to treat. FIGS. 10A and 10B show the tumor volume in mice treated with T cells expressing Logic Gates 1-5, PSMA CAR+DNR, RNP or PBS generated from T cells from either donor 1 (FIG. 10A) or donor 2 (FIG. 10B). LG 1, LG 2, LG 4, and LG 5 T cells reduced PC3 tumor volume in mice after treatment. At the 0.3×10⁶ dose LG 1 was able to clear tumors while LG 3 was not able to inhibit tumor growth. Similar anti-tumor potency trends were observed for LG 1 T cells after mice were treated with a lower 0.75×10⁵ dose in the same model (FIGS. 11A and 11B). Two doses were tested in order to measure dose response of LG 1 T cells and compare to PSMA CAR+DNR T cells. LG 1 IC showed a 4-fold increase in anti-tumor potency as compared to the PSMA CAR+ DNR positive control (FIGS. 12A and 12B). Kaplan Meier probability of survival curves for mice treated with the indicated LG T cells or controls in the PC3 tumor model are provided in FIGS. 13A and 13B. Comparison of the Kaplan Meier curves in FIGS. 13A and 13B show that mice treated with LG 1 integrated circuit T cells had a more durable response compared to PSMA CAR+ DNR positive control.

Direct comparison of the LG 1 T cells to the PSMA CAR+DNR demonstrated a four-fold increase in potency (FIGS. 12A and 12B). FIGS. 13A and 13B showed that LG 1 exhibited a more durable response as compared to the positive control PSMA CAR+DNR. These in vivo studies indicated that LG 1 is most favorable in terms of selectivity and potency of all of the logic gates used in the T cells tested.

### Example 3: In Vivo Efficacy of PSMA and PSCA Logic Gate T Cells in mCRPC Intratibial PC3 Tumor Model

Nearly 80% of mCRPC patients develop bone metastases leading to morbidity in these patients. Thus, the ability of the PSMA and PSCA Logic Gate T cells to penetrate bone tumors and induce tumor regression in a metastatic castration-resistant prostate cancer (mCRPC) bone tumor murine model was evaluated.

### Materials and Methods

### PC3 Intratibial Model and Ex Vivo Analysis

9-week-old male NSG MHC DKO mice were initially anesthetized using 2% isoflurane in an oxygen chamber. Following anesthesia, their eyes were lubricated with ointment. Each mouse was then individually transferred to a heat pump water heater and nose cone, maintaining 2% isoflurane anesthesia. Subsequently, the left tibia of each mouse was shaved and sanitized with 70% alcohol wipes. Using a tuberculin syringe with a 27 gauge needle, a hole was drilled into the knee joint through the tibia using rotation drilling techniques. With the same syringe, 0.1e6 PC3^{PSCA/PSMA} cells (human PC3 cells engineered to express PSCA and PSMA) in 20 μl PBS were injected very slowly into the tibia of the mouse. After waiting 10-15 seconds, the syringe was withdrawn slowly to minimize leaks. 5 mg/kg of analgesic meloxicam was subcutaneously injected in the midback region. The mouse was then placed in an observation chamber until it regained consciousness and then was put back in the cage.

On day 4, bioluminescent imaging was performed on an IVIS Spectrum (Caliper Life Science) and quantified using Living Image 4.0 software. D-luciferin (firefly) potassium salt solution (Perkin Elmers) was prepared (15 mg/ml) and injected intraperitoneally (0.139 g luciferin per kg body weight). Total flux (photons per second) values were obtained by imaging mice until peak radiance was achieved and quantified with Living Image 4.8 software. Tumors were randomized such that the mean tumor flux per group was 2.102×10⁷ with SEM 1.14×10⁷.

Seven mice/group were injected intravenously with a single dose of 0.3×10⁶, 1×10⁶ or 3×10⁶ of PrimeR+ ICT cells expressing LG 1, RNP or PBS control. PrimeR+ ICT cells were engineered from two donors. RNP was administered to match the total T cells in the 3×10⁶ PrimeR+ ICT cell dose group. Total tumor flux and body weight were recorded twice weekly until day 17. The study was terminated on day 21. Data represents a two donor study with 7 mice per group, mean and SEM plotted.

On Day 9 and Day 17, tumor bearing tibias were surgically dissected from 3 animals/cohort using aseptic techniques. Day 17 matched naïve non-tumor bearing tibias from three mice per group were also surgically dissected. Each individual tibia was placed per well in a 6-well plate containing 2 ml RPMI. The muscle around the tibia was cleaned with sterile scalpel. The bone epiphysis was cut open and the bone marrow was flushed using a 1 mL syringe with a 27 gauge needle attached. Single cell suspensions for the bone marrow were obtained with gentle pipetting and filtered through 40 μm filters to remove large tissue debris and muscle pieces. Any remaining cells in the well were collected by a subsequent wash step with addition 2 ml of RPMI medium through the 40 μm filter. The falcon tube was spun at 400 g, 10 min to collect the bone marrow cells. Single cells were suspended in a final volume of 200 μl FACS staining buffer, transferred into a 96 well plate and stained and quantified for total T cells and LG 1 priming receptor expression by flow cytometry via an Attune 5. Data is representative of a two donor study with 7 mice per group, mean and SEM plotted.

### Results

### PC3 Intratibial Model

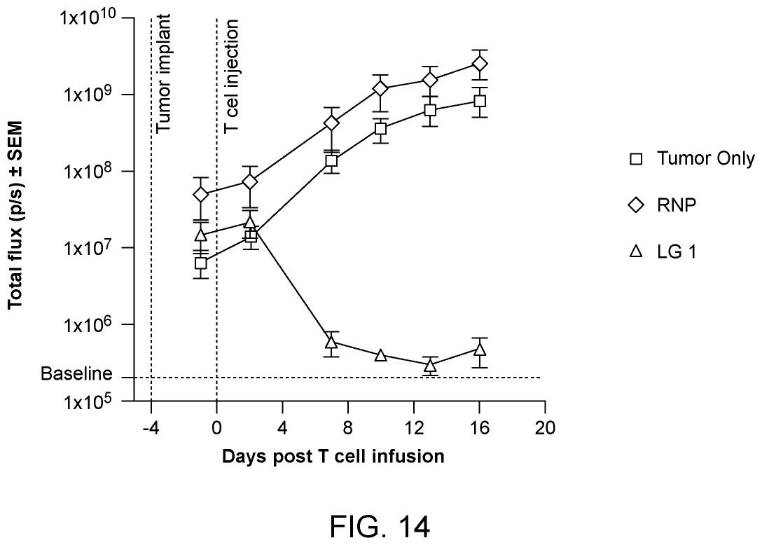

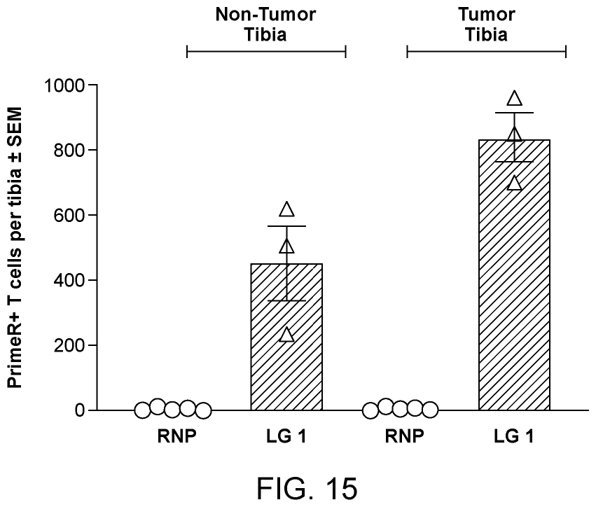

To assess if LG 1 ICT cells could penetrate bone tumors and induce tumor regression, a PC3 intratibial tumor model was used. FIG. 14 shows the total tumor bioluminescence flux (photon/second) in mice treated with 3×10⁶ T cells expressing Logic Gate 1 (LG 1 ICT cells), RNP or PBS. As shown in FIG. 14, the LG 1 ICT cells reduced PC3 tumor bioluminescence flux (e.g., reduced the tumor cell burden) in mice as compared to the RNP or PBS control groups. The ex-vivo flow analysis showed increased trafficking of the LG 1 ICT cells to the tumor-bearing tibia on day 15 (FIG. 15) compared to the non-tumor bearing tibia. Thus, LG 1 ICT cells demonstrated bone metastasis trafficking and anti-tumor potency in an mCRPC intratibial PC3 tumor model expressing PSCA and PSMA.

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

SEQUENCE LISTING

| SEQ | | |
|---|---|---|
| ID NO | Name | Sequence |
| 1 | PSCA amino | MAGLALQPGTALLCYSCKAQVSNEDCLQVENCTQLGEQCWTARIRAVGLLTVISKGCS |
| | acid | LNCVDDSQDYYVGKKNITCCDTDLCNASGAHALQPAAAILALLPALGLLLWGPGQL |
| | (UNIPROT | |
| | O43653) | |
| 2 | PSMA amino | MWNLLHETDSAVATARRPRWLCAGALVLAGGFFLLGFLFGWFIKSSNEATNITPKHNM |
| | acid | KAFLDELKAENIKKFLYNFTQIPHLAGTEQNFQLAKQIQSQWKEFGLDSVELAHYDVL |
| | (UNIPROT | LSYPNKTHPNYISIINEDGNEIFNTSLFEPPPPGYENVSDIVPPFSAFSPQGMPEGDL |
| | Q04609) | VYVNYARTEDFFKLERDMKINCSGKIVIARYGKVFRGNKVKNAQLAGAKGVILYSDPA |
| | | DYFAPGVKSYPDGWNLPGGGVQRGNILNLNGAGDPLTPGYPANEYAYRRGIAEAVGLP |
| | | SIPVHPIGYYDAQKLLEKMGGSAPPDSSWRGSLKVPYNVGPGFTGNESTQKVKMHIHS |
| | | TNEVTRIYNVIGTLRGAVEPDRYVILGGHRDSWVFGGIDPQSGAAVVHEIVRSFGTLK |
| | | KEGWRPRRTILFASWDAEEFGLLGSTEWAEENSRLLQERGVAYINADSSIEGNYTLRV |
| | | DCTPLMYSLVHNLTKELKSPDEGFEGKSLYESWTKKSPSPEFSGMPRISKLGSGNDFE |
| | | VFFQRLGIASGRARYTKNWETNKFSGYPLYHSVYETYELVEKFYDPMFKYHLTVAQVR |
| | | GGMVFELANSIVLPFDCRDYAVVLRKYADKIYSISMKHPQEMKTYSVSFDSLFSAVKN |
| | | FTEIASKFSERLQDEDKSNPIVLRMMNDQLMFLERAFIDPLGLPDRPFYRHVIYAPSS |
| | | HNKYAGESFPGIYDALFDIESKVDPSKAWGEVKRQIYVAAFTVQAAAETLSEVA |
| 3 | FAS mRNA | CTCTTCTCCCGCGGGTTGGTGGACCCGCTCAGTACGGAGTTGGGGAAGCTCTTTCACT |
| | NCBI | TCGGAGGATTGCTCAACAACCATGCTGGGCATCTGGACCCTCCTACCTCTGGTTCTTA |
| | NM_000043.6 | CGTCTGTTGCTAGATTATCGTCCAAAAGTGTTAATGCCCAAGTGACTGACATCAACTC |
| | | CAAGGGATTGGAATTGAGGAAGACTGTTACTACAGTTGAGACTCAGAACTTGGAAGGC |
| | | CTGCATCATGATGGCCAATTCTGCCATAAGCCCTGTCCTCCAGGTGAAAGGAAAGCTA |
| | | GGGACTGCACAGTCAATGGGGATGAACCAGACTGCGTGCCCTGCCAAGAAGGGAAGGA |
| | | GTACACAGACAAAGCCCATTTTTCTTCCAAATGCAGAAGATGTAGATTGTGTGATGAA |
| | | GGACATGGCTTAGAAGTGGAAATAAACTGCACCCGGACCCAGAATACCAAGTGCAGAT |
| | | GTAAACCAAACTTTTTTTGTAACTCTACTGTATGTGAACACTGTGACCCTTGCACCAA |
| | | ATGTGAACATGGAATCATCAAGGAATGCACACTCACCAGCAACACCAAGTGCAAAGAG |
| | | GAAGGATCCAGATCTAACTTGGGGTGGCTTTGTCTTCTTCTTTTGCCAATTCCACTAA |
| | | TTGTTTGGGTGAAGAGAAAGGAAGTACAGAAAACATGCAGAAAGCACAGAAAGGAAAA |
| | | CCAAGGTTCTCATGAATCTCCAACTTTAAATCCTGAAACAGTGGCAATAAATTTATCT |
| | | GATGTTGACTTGAGTAAATATATCACCACTATTGCTGGAGTCATGACACTAAGTCAAG |
| | | TTAAAGGCTTTGTTCGAAAGAATGGTGTCAATGAAGCCAAAATAGATGAGATCAAGAA |
| | | TGACAATGTCCAAGACACAGCAGAACAGAAAGTTCAACTGCTTCGTAATTGGCATCAA |
| | | CTTCATGGAAAGAAAGAAGCGTATGACACATTGATTAAAGATCTCAAAAAAGCCAATC |
| | | TTTGTACTCTTGCAGAGAAAATTCAGACTATCATCCTCAAGGACATTACTAGTGACTC |
| | | AGAAAATTCAAACTTCAGAAATGAAATCCAAAGCTTGGTCTAGAGTGAAAAACAACAA |
| | | ATTCAGTTCTGAGTATATGCAATTAGTGTTTGAAAAGATTCTTAATAGCTGGCTGTAA |
| | | ATACTGCTTGGTTTTTTACTGGGTACATTTTATCATTTATTAGCGCTGAAGAGCCAAC |
| | | ATATTTGTAGATTTTTAATATCTCATGATTCTGCCTCCAAGGATGTTTAAAATCTAGT |
| | | TGGGAAAACAAACTTCATCAAGAGTAAATGCAGTGGCATGCTAAGTACCCAAATAGGA |
| | | GTGTATGCAGAGGATGAAAGATTAAGATTATGCTCTGGCATCTAACATATGATTCTGT |
| | | AGTATGAATGTAATCAGTGTATGTTAGTACAAATGTCTATCCACAGGCTAACCCCACT |
| | | CTATGAATCAATAGAAGAAGCTATGACCTTTTGCTGAAATATCAGTTACTGAACAGGC |
| | | AGGCCACTTTGCCTCTAAATTACCTCTGATAATTCTAGAGATTTTACCATATTTCTAA |
| | | ACTTTGTTTATAACTCTGAGAAGATCATATTTATGTAAAGTATATGTATTTGAGTGCA |
| | | GAATTTAAATAAGGCTCTACCTCAAAGACCTTTGCACAGTTTATTGGTGTCATATTAT |
| | | ACAATATTTCAATTGTGAATTCACATAGAAAACATTAAATTATAATGTTTGACTATTA |
| | | TATATGTGTATGCATTTTACTGGCTCAAAACTACCTACTTCTTTCTCAGGCATCAAAA |
| | | GCATTTTGAGCAGGAGAGTATTACTAGAGCTTTGCCACCTCTCCATTTTTGCCTTGGT |
| | | GCTCATCTTAATGGCCTAATGCACCCCCAAACATGGAAATATCACCAAAAAATACTTA |
| | | ATAGTCCACCAAAAGGCAAGACTGCCCTTAGAAATTCTAGCCTGGTTTGGAGATACTA |
| | | ACTGCTCTCAGAGAAAGTAGCTTTGTGACATGTCATGAACCCATGTTTGCAATCAAAG |
| | | ATGATAAAATAGATTCTTATTTTTCCCCCACCCCCGAAAATGTTCAATAATGTCCCAT |
| | | GTAAAACCTGCTACAAATGGCAGCTTATACATAGCAATGGTAAAATCATCATCTGGAT |
| | | TTAGGAATTGCTCTTGTCATACCCCCAAGTTTCTAAGATTTAAGATTCTCCTTACTAC |
| | | TATCCTACGTTTAAATATCTTTGAAAGTTTGTATTAAATGTGAATTTTAAGAAATAAT |
| | | ATTTATATTTCTGTAAATGTAAACTGTGAAGATAGTTATAAACTGAAGCAGATACCTG |
| | | GAACCACCTAAAGAACTTCCATTTATGGAGGATTTTTTTGCCCCTTGTGTTTGGAATT |
| | | ATAAAATATAGGTAAAAGTACGTAATTAAATAATGTTTTTGGTATTTCTGGTTTTCTC |
| | | TTTTTTGGTAGGGGCTTGCTTTTTGGTTTTGTCTTCCTTTTCTCTAACTGATGCTAAA |
| | | TATAACTTGTCTTTAATGCTTCTTGGATCCCTTAGAAGGTACTTCCTTTTTAACCTTA |
| | | ACCCTTTTAGTAGTTAAATAATTATTTCCATAGGTTGCTATTGCCAAGAAGACCTCTT |
| | | CCAAACAGCACATGATTATTCGTCAAACAGTTTCGTATTCCAGATACTGGAATGTGGA |
| | | TAAGAAAGTATACATTTCAAGGGGTAGGTTTTATTATTAAGAAAGCCAAATGAGGATT |
| | | TTGAAATATTCTTTCCTGCATATTATCCATTCTAGCTACATGCTGGCCAGTGGGCCAC |
| | | CTTTCTTTTCTGCAATTTAATGCTAGTAATATATTCTATTTAACCCATGAGTCCCAAA |
| | | GTATTAGCATTTCAACATGTAAGCATGTCGGTAAGATAGTTGTGCTTTGCTTAGGGTT |
| | | CCCTCCTGTGTTATGGTCTGGAAAGTGTCTTTAGGCAGAAAGTCTGAGTGATCACAGG |
| | | GTTCACTCATTAATTTCTCTTTTCTGAGCCATCATAGTCTGTGCTGTCTGCTCTCCAG |
| | | TTTTCTATTTCTAGACAGAAGTAGGGCAAGTTAGGTACTAGTTATTCTTCATGGCCAG |
| | | AAGTGCAAGTTCTACTTTGCAAGACAAGATTAAGTTAGAGAACACCCTATTCCACTTT |
| | | GGTGAACTCAGAGCAAGAACTTTGAGTTCCTTTGGGAGGAAGACAGTGGAGAAGTCTT |
| | | TGTACTTGGTGATGTGGTTTTTTTCCTCATGGCTTCACCTAGTGGCCCCAAGCATGAC |
| | | TTCTCCCATGTCAATGAGCACAGCCACATTCCCGAGTTGAGGTGACCCCACGGTCCAG |
| | | AATCATCCTCATTCTGGTGAACCTGGTTCTCTTTGTGGTGGGCATACTGGGTAGGAGA |
| | | ATCACCCAAAGGTCACCCATGAGCTGCAGAAAAAAAGGCTATTTGCAGAAGGAGCTCA |
| | | CAGATCACATTGAAAGCATTGCATATTCAAACATCTTGGTCTTCTTTATTGGCATGCC |
| | | CACAGGGTCTTCTGACCTCTGATTAGATCAGACACTTTTTAGATATTGAATCATCAGT |
| | | TTCTGTACAACTATCTGAATAAGGTATATAATCAATGAAATTTAGAATTTTTTTCTAT |
| | | GCTTACTCCTGATTGGTAATTTGTTTGGGTTTAGAATTCTATACAAGGCCATTTGTAA |
| | | TTTTCCTCAGCACTTTAAAAATATTAAACCATGTTTTCTTAA |
| 4 | TGF-β | ACTCGCGCGCACGGAGCGACGACACCCCCGCGCGTGCACCCGCTCGGGACAGGAGCCG |
| | Receptor 2 | GACTCCTGTGCAGCTTCCCTCGGCCGCCGGGGGCCTCCCCGCGCCTCGCCGGCCTCCA |
| | (TGFBR2) | GGCCCCCTCCTGGCTGGCGAGCGGGCGCCACATCTGGCCCGCACATCTGCGCTGCCGG |
| | cDNA | CCCGGCGCGGGGTCCGGAGAGGGCGCGGCGCGGAGGCGCAGCCAGGGGTCCGGGAAGG |
| | | CGCCGTCCGCTGCGCTGGGGGCTCGGTCTATGACGAGCAGCGGGGTCTGCCATGGGTC |
| | | GGGGGCTGCTCAGGGGCCTGTGGCCGCTGCACATCGTCCTGTGGACGCGTATCGCCAG |
| | | CACGATCCCACCGCACGTTCAGAAGTCGGTTAATAACGACATGATAGTCACTGACAAC |
| | | AACGGTGCAGTCAAGTTTCCACAACTGTGTAAATTTTGTGATGTGAGATTTTCCACCT |
| | | GTGACAACCAGAAATCCTGCATGAGCAACTGCAGCATCACCTCCATCTGTGAGAAGCC |
| | | ACAGGAAGTCTGTGTGGCTGTATGGAGAAAGAATGACGAGAACATAACACTAGAGACA |
| | | GTTTGCCATGACCCCAAGCTCCCCTACCATGACTTTATTCTGGAAGATGCTGCTTCTC |
| | | CAAAGTGCATTATGAAGGaaaaaaaaaaGCCTGGTGAGACTTTCTTCATGTGTTCCTG |
| | | TAGCTCTGATGAGTGCAATGACAACATCATCTTCTCAGAAGAATATAACACCAGCAAT |
| | | CCTGACTTGTTGCTAGTCATATTTCAAGTGACAGGCATCAGCCTCCTGCCACCACTGG |
| | | GAGTTGCCATATCTGTCATCATCATCTTCTACTGCTACCGCGTTAACCGGCAGCAGAA |
| | | GCTGAGTTCAACCTGGGAAACCGGCAAGACGCGGAAGCTCATGGAGTTCAGCGAGCAC |
| | | TGTGCCATCATCCTGGAAGATGACCGCTCTGACATCAGCTCCACGTGTGCCAACAACA |
| | | TCAACCACAACACAGAGCTGCTGCCCATTGAGCTGGACACCCTGGTGGGGAAAGGTCG |
| | | CTTTGCTGAGGTCTATAAGGCCAAGCTGAAGCAGAACACTTCAGAGCAGTTTGAGACA |
| | | GTGGCAGTCAAGATCTTTCCCTATGAGGAGTATGCCTCTTGGAAGACAGAGAAGGACA |
| | | TCTTCTCAGACATCAATCTGAAGCATGAGAACATACTCCAGTTCCTGACGGCTGAGGA |
| | | GCGGAAGACGGAGTTGGGGAAACAATACTGGCTGATCACCGCCTTCCACGCCAAGGGC |
| | | AACCTACAGGAGTACCTGACGCGGCATGTCATCAGCTGGGAGGACCTGCGCAAGCTGG |
| | | GCAGCTCCCTCGCCCGGGGGATTGCTCACCTCCACAGTGATCACACTCCATGTGGGAG |
| | | GCCCAAGATGCCCATCGTGCACAGGGACCTCAAGAGCTCCAATATCCTCGTGAAGAAC |
| | | GACCTAACCTGCTGCCTGTGTGACTTTGGGCTTTCCCTGCGTCTGGACCCTACTCTGT |
| | | CTGTGGATGACCTGGCTAACAGTGGGCAGGTGGGAACTGCAAGATACATGGCTCCAGA |
| | | AGTCCTAGAATCCAGGATGAATTTGGAGAATGTTGAGTCCTTCAAGCAGACCGATGTC |
| | | TACTCCATGGCTCTGGTGCTCTGGGAAATGACATCTCGCTGTAATGCAGTGGGAGAAG |
| | | TAAAAGATTATGAGCCTCCATTTGGTTCCAAGGTGCGGGAGCACCCCTGTGTCGAAAG |
| | | CATGAAGGACAACGTGTTGAGAGATCGAGGGCGACCAGAAATTCCCAGCTTCTGGCTC |
| | | AACCACCAGGGCATCCAGATGGTGTGTGAGACGTTGACTGAGTGCTGGGACCACGACC |
| | | CAGAGGCCCGTCTCACAGCCCAGTGTGTGGCAGAACGCTTCAGTGAGCTGGAGCATCT |
| | | GGACAGGCTCTCGGGGAGGAGCTGCTCGGAGGAGAAGATTCCTGAAGACGGCTCCCTA |
| | | AACACTACCAAATAGCTCTTCTGGGGCAGGCTGGGCCATGTCCAAAGAGGCTGCCCCT |
| | | CTCACCAAAGAACAGAGGCAGCAGGAAGCTGCCCCTGAACTGATGCTTCCTGGAAAAC |
| | | CAAGGGGGTCACTCCCCTCCCTGTAAGCTGTGGGGATAAGCAGAAACAACAGCAGCAG |
| | | GGAGTGGGTGACATAGAGCATTCTATGCCTTTGACATTGTCATAGGATAAGCTGTGTT |
| | | AGCACTTCCTCAGGAAATGAGATTGATTTTTACAATAGCCAATAACATTTGCACTTTA |
| | | TTAATGCCTGtatataaatatgaatagctatgttttatatatatatatatatatctat |
| | | atatgtctatagctctatatatataGCCATACCTTGAAAAGAGACAAGGAAAAACATC |
| | | AAATATTCCCAGGAAATTGGTTTTATTGGAGAACTCCAGAACCAAGCAGAGAAGGAAG |
| | | GGACCCATGACAGCATTAGCATTTGACAATCACACATGCAGTGGTTCTCTGACTGTAA |
| | | AACAGTGAACTTTGCATGAGGAAAGAGGCTCCATGTCTCACAGCCAGCTATGACCACA |
| | | TTGCACTTGCTTTTGCAAAATAATCATTCCCTGCCTAGCACTTCTCTTCTGGCCATGG |
| | | AACTAAGTACAGTGGCACTGTTTGAGGACCAGTGTTCCCGGGGTTCCTGTGTGCCCTT |
| | | ATTTCTCCTGGACTTTTCATTTAAGCTCCAAGCCCCAAATCTGGGGGGCTAGTTTAGA |
| | | AACTCTCCCTCAACCTAGTTTAGAAACTCTACCCCATCTTTAATACCTTGAATGTTTT |
| | | GAACCCCACTTTTTACCTTCATGGGTTGCAGAAAAATCAGAACAGATGTCCCCATCCA |
| | | TGCGATTGCCCCACCATCTACTAATGAAAAATTGTTCTTTTTTTCATCTTTCCCCTGC |
| | | ACTTATGTTACTATTCTCTGCTCCCAGCCTTCATCCTTTTCTAAAAAGGAGCAAATTC |
| | | TCACTCTAGGCTTTATCGTGTTTACTTTTTCATTACACTTGACTTGATTTTCTAGTTT |
| | | TCTATACAAACACCAATGGGTTCCATCTTTCTGGGCTCCTGATTGCTCAAGCACAGTT |
| | | TGGCCTGATGAAGAGGATTTCAACTACACAATACTATCATTGTCAGGACTATGACCTC |
| | | AGGCACTCTAAACATAtgttttgtttggtcagcacagcgtttcaaaaagtgaagccac |
| | | tttataaatatttggagattttgcaggaaaatctggatccccagGTAAGGATAGCAGA |
| | | TGGTTTTCAGTTATCTCCAGTCCACGTTCACAAAATGTGAAGGTGTGGAGACACTTAC |
| | | AAAGCTGCCTCACTTCTCACTGTAAACATTAGCTCTTTCCACTGCCTACCTGGACCCC |
| | | AGTCTAGGAATTAAATCTGCACCTAACCAAGGTCCCTTGTAAGAAATGTCCATTCAAG |
| | | CAGTCATTCTCTGGGTATATAATATGATTTTGACTACCTTATCTGGTGTTAAGATTTG |
| | | AAGTTGGCCTTTTATTGGACTAAAGGGGAACTCCTTTAAGGGTCTCAGTTAGCCCAAG |
| | | TTTCTTTTGCTTATATGTTAATAGTTTTACCCTCTGCATTGGAGAGAGGAGTGCTTTA |
| | | CTCCAAGAAGCTTTCCTCATGGTTACCGTTCTCTCCATCATGCCAGCCTTCTCAACCT |
| | | TTGCAGAAATTACTAGAGAGGATTTGAATGTGGGACACAAAGGTCCCATTTGCAGTTA |
| | | GAAAATTTGTGTCCACAAGGACAAGAACAAAGTATGAGCTTTAAAACTCCATAGGAAA |
| | | CTTGTTAATCAACAAAGAAGTGTTAATGCTGCAAGTAATCTCTTTTTTAAAACTTTTT |
| | | GAAGCTACTTATTTTCAGCCAAATAGGAATATTAGAGAGGGACTGGTAGTGAGAATAT |
| | | CAGCTCTGTTTGGATGGTGGAAGGTCTCATTTTATTGAGATTTTTAAGATACATGCAA |
| | | AGGTTTGGAAATAGAACCTCTAGGCACCCTCCTCAGTGTGGGTGGGCTGAGAGTTAAA |
| | | GACAGTGTGGCTGCAGTAGCATAGAGGCGCCTAGAAATTCCACTTGCACCGTAGGGCA |
| | | TGCTGATACCATCCCAATAGCTGTTGCCCATTGACCTCTAGTGGTGAGTTTCTAGAAT |
| | | ACTGGTCCATTCATGAGATATTCAAGATTCAAGAGTATTCTCACTTCTGGGTTATCAG |
| | | CATAAACTGGAATGTAGTGTCAGAGGATACTGTGGCTTGTTTTGTTTATGtttttttt |
| | | tCTTATTCAAGAAAAAAGACCAAGGAATAACATTCTGTAGTTCCTAAAAATACTGACT |
| | | TTTTTCACTACTATACATAAAGGGAAAGTTTTATTCTTTTATGGAACACTTCAGCTGT |
| | | ACTCATGTATTAAAATAGGAATGTGAATGCTATATACTCTTTTTATATCAAAAGTCTC |
| | | AAGCACTTATTTTTATTCTATGCATTGTTTGTCTTTTACATAAATAAAATGTTTATTA |
| | | GATTGAATAAAGCAAAATACTCAGGTGAGCATCCTGCCTCCTGTTCCCATTCCTAGTA |
| | | GCTAAA |
| 5 | sgRNA 94 | GAGCCATGCTTGGCTTACGA |
| 6 | BGH poly A | cgactgtgccttctagttgccagccatctgttgtttgcccctcccccgtgccttcctt |
| | | gaccctggaaggtgccactcccactgtcctttcctaataaaatgaggaaattgcatcg |
| | | cattgtctgagtaggtgtcattctattctggggggtggggtggggcaggacagcaagg |
| | | gggaggattgggaagacaatagcaggcatgctggggatgcggtgggctctatgg |
| 7 | 6X HNF1α | gttaataattaacatatatgttaatcattaacatatagttaattattaaccgctatgt |
| | response | taatgattaacaacggttaataattaacatatatgttaatcattaacatata |
| | elements + | tctagagggtatataatgggggcca |
| | YB-TATA | |
| | promoter | |
| 8 | CD8α signal | MALPVTALLLPLALLLHAARP |
| | sequence | |
| 9 | CAR Hinge | TTTPAPRPPTPAPTIASQPLSLRPEACRPAAGGAVHTRGLDFACD |
| | CD8α | |
| 10 | CAR CD8α | IYIWAPLAGTCGVLLLSLVITLYC |
| | Trans- | |
| | membrane | |
| | Domain | |
| | (TMD) | |
| 11 | CAR Co-stim | KRGRKKLLYIFKQPFMRPVQTTQEEDGCSCRFPEEEEGGCEL |
| | 4-1BB | |
| 12 | CAR | RVKFSRSADAPAYQQGQNQLYNELNLGRREEYDVLDKRRGRDPEMGGKPRRKNPQEGL |
| | Activation | YNELQKDKMAEAYSEIGMKGERRRGKGHDGLYQGLSTATKDTYDALHMQALPPR |
| | CD3z | |
| 13 | WPRE | AATCAACCTCTGGATTACAAAATTTGTGAAAGATTGACTGGTATTCTTAACTATGTTG |
| | | CTCCTTTTACGCTATGTGGATACGCTGCTTTAATGCCTTTGTATCATGCTATTGCTTC |
| | | CCGTATGGCTTTCATTTTCTCCTCCTTGTATAAATCCTGGTTGCTGTCTCTTTATGAG |
| | | GAGTTGTGGCCCGTTGTCAGGCAACGTGGCGTGGTGTGCACTGTGTTTGCTGACGCAA |
| | | CCCCCACTGGTTGGGGCATTGCCACCACCTGTCAGCTCCTTTCCGGGACTTTCGCTTT |
| | | CCCCCTCCCTATTGCCACGGCGGAACTCATCGCCGCCTGCCTTGCCCGCTGCTGGACA |
| | | GGGGCTCGGCTGTTGGGCACTGACAATTCCGTGGTGTTGTCGGGGAAATCATCGTCCT |
| | | TTCCTTGGCTGCTCGCCTGTGTTGCCACCTGGATTCTGCGCGGGACGTCCTTCTGCTA |
| | | CGTCCCTTCGGCCCTCAATCCAGCGGACCTTCCTTCCCGCGGCCTGCTGCCGGCTCTG |
| | | CGGCCTCTTCCGCGTCTTCGCCTTCGCCCTCAGACGAGTCGGATCTCCCTTTGGGCCG |
| | | CCTCCCCGCCTG |
| 14 | SV40 poly A | AACTTGTTTATTGCAGCTTATAATGGTTACAAATAAAGCAATAGCATCACAAATTTCA |
| | | CAAATAAAGCATTTTTTTCACTGCATTCTAGTTGTGGTTTGTCCAAACTCATCAATGT |
| | | ATCTTATCATGTCTGG |
| 15 | EF1α | GGATCTGCGATCGCTCCGGTGCCCGTCAGTGGGCAGAGCGCACATCGCCCACAGTCCC |
| | promoter | CGAGAAGTTGGGGGGAGGGGTCGGCAATTGAACGGGTGCCTAGAGAAGGTGGCGCGGG |
| | | GTAAACTGGGAAAGTGATGTCGTGTACTGGCTCCGCCTTTTTCCCGAGGGTGGGGGAG |
| | | AACCGTATATAAGTGCAGTAGTCGCCGTGAACGTTCTTTTTCGCAACGGGTTTGCCGC |
| | | CAGAACACAGCTGAAGCTTCGAGGGGCTCGCATCTCTCCTTCACGCGCCCGCCGCCCT |
| | | ACCTGAGGCCGCCATCCACGCCGGTTGAGTCGCGTTCTGCCGCCTCCCGCCTGTGGTG |
| | | CCTCCtgaactgcgtccgccgtctag |
| 16 | FAS_11 | TTAAGAATCTTTTCAAACACTA |
| 17 | TGFBR2_37 | TTATGTAAAAGACAAACAATGC |
| 18 | TGFBR2_16 | TTGATTAACAAGTTTCCTATGG |
| 19 | TGFBR2_23 | TAAAAATCAATCTCATTTCCTG |
| 20 | FAS_11/TGF | CCGGATCAACGCCCTAGGTTTATGTTTGGATGAACTGACATACGCGTATCCGTCTTAA |
| | BR2_16/TGF | GAATCTTTTCAAACACTAGTAGTGAAATATATATTAAACTAGTGTTTGAAAAGATTCT |
| | BR2_23/TGF | TATTACGGTAACGCGGAATTCGCAACTATTTTATCAATTTTTTGCGTCGACACTTCAA |
| | BR2_37 | GGGGCTTGCGGCCGCAACCATCTCCATGGCTGTTTGAATGAGGCTTCAGTACTTTACA |
| | shRNA | GAATCGTTGCCTGCACATCTTGGAAACACTTGCTGGGATTACTTCGACTTCTTAACCC |
| | cassette | AACAGAAGGCTCGAGAAGGTATATTGCTGTTGACAGTGAGCGCCATAGGAAACTTGTT |
| | | AATCATTAGTGAAGCCACAGATGTATTGATTAACAAGTTTCCTATGGTGCCTACTGCC |
| | | TCGGACTTCAAGGGGCTAGAATTCGAGCAATTATCTTGTTTACTAAAACTGAATACCT |
| | | TGCTATCTCTTTGATACATTTTTACAAAGCTGAATTAAAATGGTATAAATTAAATCAC |
| | | TTTGACGGTGACTTAGGAGTATGCCGGATCAACGCCCTAGGTTTATGTTTGGATGAAC |
| | | TGACATACGCGTATCCGTCTAAAAATCAATCTCATTTCCTGGTAGTGAAATATATATT |
| | | AAACCAGGAAATGAGATTGATTTTTTTACGGTAACGCGGAATTCGCAACTATTTTATC |
| | | AATTTTTTGCGTCGACGACTGTGACAGCAGAGTATGCCGGATCAACGCCCTAGGTTTA |
| | | TGTTTGGATGAACTGACATACGCGTATCCGTCTTATGTAAAAGACAAACAATGCGTAG |
| | | TGAAATATATATTAAACGCATTGTTTGTCTTTTACATATTACGGTAACGCGGAATTCG |
| | | CAACTATTTTATCAATTTTTTGCGTCGACCGGAACTATCTTGAAGAGTA |
| 21 | primeR CD8α | TTTPAPRPPTPAPTIASQPLSLRPEAC |
| | Hinge | |
| 22 | primeR | FMYVAAAAFVLLFFVGCGVLLS |
| | Notch1_ | |
| | TMD | |
| 23 | primeR | RKRRRQHGQLWFPEGFKVSEASKKKRREPLGEDSVGLKPLKNA |
| | Notch1_ | |
| | Stop- | |
| | Transfer- | |
| | Sequence | |
| | (JMD) | |
| 24 | primeR | MVSKLSQLQTELLAALLESGLSKEALLQALGEPGPYLLAGEGPLDKGESCGGGRGELA |
| | HNF1a | ELPNGLGETRGSEDETDDDGEDFTPPILKELENLSPEEAAHQKAVVETLLQEDPWRVA |
| | DNA-binding | KMVKSYLQQHNIPQREVVDTTGLNQSHLSQHLNKGTPMKTQKRAALYTWYVRKQREVA |
| | domain | QQFTHAGQGGLIEEPTGDELPTKKGRRNRFKWGPASQQILFQAYERQKNPSKEERETL |
| | (DBD) | VEECNRAECIQRGVSPSQAQGLGSNLVTEVRVYNWFANRRKEEAFRHKLAM |
| 25 | primeR p65 | DEFPTMVFPSGQISQASALAPAPPQVLPQAPAPAPAPAMVSALAQAPAPVPVLAPGPP |
| | Trans- | QAVAPPAPKPTQAGEGTLSEALLQLQFDDEDLGALLGNSTDPAVFTDLASVDNSEFQQ |
| | activation | LLNQGIPVAPHTTEPMLMEYPEAITRLVTGAQRPPDPAPAPLGAPGLPNGLLSGDEDF |
| | domain | SSIADMDFSALLSQISS |
| | (TAD) | |
| 26 | primeR | MVSKLSQLQTELLAALLESGLSKEALLQALGEPGPYLLAGEGPLDKGESCGGGRGELA |
| | HNF1a-p65 | ELPNGLGETRGSEDETDDDGEDFTPPILKELENLSPEEAAHQKAVVETLLQEDPWRVA |
| | TAD | KMVKSYLQQHNIPQREVVDTTGLNQSHLSQHLNKGTPMKTQKRAALYTWYVRKQREVA |
| | | QQFTHAGQGGLIEEPTGDELPTKKGRRNRFKWGPASQQILFQAYERQKNPSKEERETL |
| | | VEECNRAECIQRGVSPSQAQGLGSNLVTEVRVYNWFANRRKEEAFRHKLAMTCRDEFP |
| | | TMVFPSGQISQASALAPAPPQVLPQAPAPAPAPAMVSALAQAPAPVPVLAPGPPQAVA |
| | | PPAPKPTQAGEGTLSEALLQLQFDDEDLGALLGNSTDPAVFTDLASVDNSEFQQLLNQ |
| | | GIPVAPHTTEPMLMEYPEAITRLVTGAQRPPDPAPAPLGAPGLPNGLLSGDEDFSSIA |
| | | DMDFSALLSQIS |
| 27 | CD34 ECD | GAGCTGCCCACACAAGGCACATTCAGCAACGTGTCCACCAACGTGTCC |
| 28 | SPA Cys | GAGCTGTGCGGCGGC |
| | linker | |
| 29 | SPA | GCTATCGTGGTGCCCGTGTGCTTGGCCTTCCTGCTGACCACCCTGCTGGGCGTGCTGT |
| | gp 130_TMBD | TCTGCTTC |
| 30 | SPA | AACAAGCGGGACCTGATTAAGAAGCACATCTGGCCCAACGTGCCCGACCCTAGCAAGA |
| | gp130_ | GCCACATCGCCCAGTGGAGCCCTCACACACCTCCCAGGCACAACTTCAACAGCAAGGA |
| | Intra- | CCAGATGTACTCCGACGGCAACTTCACCGACGTGTCCGTGGTGGAGATCGAGGCCAAC |
| | cellular | GACAAGAAGCCCTTCCCTGAGGACCTGAAGTCCCTGGACCTGTTCAAGAAGGAGAAGA |
| | domain (ICD) | TCAACACCGAGGGACACAGCAGCGGCATCGGCGGCTCTAGCTGTATGAGCAGCAGCAG |
| | | GCCCAGCATCTCCAGCAGCGACGAGAACGAGAGCAGCCAGAACACCAGCAGCACCGTG |
| | | CAGTACAGCACCGTGGTGCACAGCGGCTACAGGCACCAGGTGCCATCAGTGCAAGTGT |
| | | TCAGCCGATCCGAGTCCACCCAGCCTCTGCTGGACAGCGAGGAGAGGCCCGAGGACCT |
| | | GCAACTGGTGGACCACGTGGACGGAGGCGACGGCATCCTGCCTAGGCAGCAATACTTC |
| | | AAGCAGAATTGCAGCCAGCACGAGAGCAGCCCTGACATCAGCCACTTCGAGAGGAGCA |
| | | AGCAAGTGTCCAGCGTGAACGAGGAGGACTTCGTGAGGCTGAAGCAGCAGATCAGCGA |
| | | CCACATCAGCCAGAGCTGCGGCAGCGGCCAGATGAAGATGTTCCAGGAAGTGTCCGCC |
| | | GCCGACGCCTTCGGCCCTGGCACAGAAGGACAAGTGGAGAGGTTCGAAACCGTGGGCA |
| | | TGGAGGCCGCCACCGACGAGGGAATGCCCAAGAGCTACCTGCCTCAGACCGTGCGGCA |
| | | GGGCGGCTACATGCCTCAG |
| 31 | GH1 poly A | CGGGTGGCATCCCTGTGACCCCTCCCCAGTGCCTCTCCTGGCCCTGGAAGTTGCCACT |
| | | CCAGTGCCCACCAGCCTTGTCCTAATAAAATTAAGTTGCATCATTTTGTCTGACTAGG |
| | | TGTCCTTCTATAATATTATGGGGTGGAGGGGGGTGGTATGGAGCAAGGGGCAAGTTGG |
| | | GAAGACAACCTGTAGGGCCTGCGGGGTCTATTGGGAACCAAGCTGGAGTGCAGTGGCA |
| | | CAATCTTGGCTCACTGCAATCTCCGCCTCCTGGGTTCAAGCGATTCTCCTGCCTCAGC |
| | | CTCCCGAGTTGTTGGGATTCCAGGCATGCATGACCAGGCTCAGCTAATTTTTGTTTTT |
| | | TTGGTAGAaACGGGGTTTCACCATATTGGCCAGGCTGGTCTCCAACTCCTAATCTCAG |
| | | GTGATCTACCCACCTTGGCCTCCCAAATTGCTGGGATTACAGGCGTGAACCACTGCTC |
| | | CCttccctgtccttc |
| 32 | PSMA scFv | QVQLVESGGGVVQPGRSLRLSCAASGFIFSSYGMHWVRQAPGKGLEWVTIIWYDGSNR |
| | | YYADSVKGRFTISRDNSKNTLYLQMNSLRAEDTAVYYCARASGDFYNYFMDVWGKGTT |
| | | VTVSSGGGGSGGGGSGGGGSEIVMTQSPATLSVSPGERATLSCRASQSVSSNLAWYQQ |
| | | KPGQAPRLLIYGASTRATGIPARFSGSGSGTEFTLTISSLQSEDFAVYYCQQYNNWPP |
| | | YTFGQGTKLEIK |
| 33 | PSMA VH | QVQLVESGGGVVQPGRSLRLSCAASGFIFSSYGMHWVRQAPGKGLEWVTIIWYDGSNR |
| | | YYADSVKGRFTISRDNSKNTLYLQMNSLRAEDTAVYYCARASGDFYNYFMDVWGKGTT |
| | | VTVSS |
| 34 | PSMA VL | EIVMTQSPATLSVSPGERATLSCRASQSVSSNLAWYQQKPGQAPRLLIYGASTRATGI |
| | | PARFSGSGSGTEFTLTISSLQSEDFAVYYCQQYNNWPPYTFGQGTKLEIK |
| 35 | PSMA CDR | SYGMH |
| | H1 | |
| 36 | PSMA CDR | IIWYDGSNRYYADSVKG |
| | H2 | |
| 37 | PSMA CDR | ASGDFYNYFMDV |
| | H3 | |
| 38 | PSMA CDR | RASQSVSSNLA |
| | L1 | |
| 39 | PSMA CDR | GASTRAT |
| | L2 | |
| 40 | PSMA CDR | QQYNNWPPYT |
| | L3 | |
| 41 | PSMA scFv | CAGGTTCAGCTTGTGGAATCTGGTGGTGGCGTGGTTCAGCCTGGGAGATCTCTGCGGC |
| | nucleic acid | TGTCATGCGCTGCGTCTGGGTTCATCTTCTCCTCATACGGAATGCATTGGGTCAGACA |
| | | GGCTCCCGGCAAAGGGCTCGAGTGGGTTACAATTATCTGGTATGATGGCAGTAATCGC |
| | | TACTACGCCGATTCCGTCAAAGGGCGGTTCACCATTTCAAGGGACAATAGCAAGAACA |
| | | CTTTGTATCTCCAAATGAACTCCCTTAGAGCAGAGGACACCGCCGTGTACTATTGCGC |
| | | CCGCGCAAGCGGCGACTTCTACAATTATTTTATGGACGTCTGGGGCAAGGGCACTACG |
| | | GTGACAGTGTCCTCCGGAGGAGGAGGATCTGGAGGAGGAGGAAGCGGTGGCGGCGGGA |
| | | GTGAGATAGTAATGACCCAGTCGCCTGCCACTCTAAGCGTATCTCCCGGCGAACGGGC |
| | | TACCTTGAGTTGTAGGGCAAGTCAATCAGTGTCCTCCAATCTGGCCTGGTATCAGCAA |
| | | AAACCGGGCCAGGCTCCTAGGCTGCTGATATACGGCGCCAGCACACGTGCAACTGGGA |
| | | TTCCAGCGCGATTTAGCGGGAGCGGTTCAGGGACGGAGTTCACCTTAACAATCAGTAG |
| | | CCTGCAATCTGAGGATTTTGCCGTGTACTATTGTCAACAGTATAACAACTGGCCACCC |
| | | TACACCTTTGGCCAGGGTACAAAGCTCGAAATCAAG |
| 42 | PSMA CAR | QVQLVESGGGVVQPGRSLRLSCAASGFIFSSYGMHWVRQAPGKGLEWVTIIWYDGSNR |
| | protein full | YYADSVKGRFTISRDNSKNTLYLQMNSLRAEDTAVYYCARASGDFYNYFMDVWGKGTT |
| | sequence | VTVSSGGGGSGGGGSGGGGSEIVMTQSPATLSVSPGERATLSCRASQSVSSNLAWYQQ |
| | | KPGQAPRLLIYGASTRATGIPARFSGSGSGTEFTLTISSLQSEDFAVYYCQQYNNWPP |
| | | YTFGQGTKLEIKAAATTTPAPRPPTPAPTIASQPLSLRPEACRPAAGGAVHTRGLDFA |
| | | CDIYIWAPLAGTCGVLLLSLVITLYCKRGRKKLLYIFKQPFMRPVQTTQEEDGCSCRF |
| | | PEEEEGGCELRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHM |
| | | QALPPR |
| 43 | PSMA CAR | CAGGTTCAGCTTGTGGAATCTGGTGGTGGCGTGGTTCAGCCTGGGAGATCTCTGCGGC |
| | nucleic acid | TGTCATGCGCTGCGTCTGGGTTCATCTTCTCCTCATACGGAATGCATTGGGTCAGACA |
| | full | GGCTCCCGGCAAAGGGCTCGAGTGGGTTACAATTATCTGGTATGATGGCAGTAATCGC |
| | sequence | TACTACGCCGATTCCGTCAAAGGGCGGTTCACCATTTCAAGGGACAATAGCAAGAACA |
| | | CTTTGTATCTCCAAATGAACTCCCTTAGAGCAGAGGACACCGCCGTGTACTATTGCGC |
| | | CCGCGCAAGCGGCGACTTCTACAATTATTTTATGGACGTCTGGGGCAAGGGCACTACG |
| | | GTGACAGTGTCCTCCGGAGGAGGAGGATCTGGAGGAGGAGGAAGCGGTGGCGGCGGGA |
| | | GTGAGATAGTAATGACCCAGTCGCCTGCCACTCTAAGCGTATCTCCCGGCGAACGGGC |
| | | TACCTTGAGTTGTAGGGCAAGTCAATCAGTGTCCTCCAATCTGGCCTGGTATCAGCAA |
| | | AAACCGGGCCAGGCTCCTAGGCTGCTGATATACGGCGCCAGCACACGTGCAACTGGGA |
| | | TTCCAGCGCGATTTAGCGGGAGCGGTTCAGGGACGGAGTTCACCTTAACAATCAGTAG |
| | | CCTGCAATCTGAGGATTTTGCCGTGTACTATTGTCAACAGTATAACAACTGGCCACCC |
| | | TACACCTTTGGCCAGGGTACAAAGCTCGAAATCAAGGCGGcagcaaccacgacGCCAG |
| | | CGCCGCGACCACCAACACCGGCGCCCACCATCGCGTCGCAGCCACTGTCACTGCGCCC |
| | | AGAAGCGTGCCGGCCAGCGGCGGGGGGCGCAGTGCACACGAGGGGGCTGGACTTCGCC |
| | | TGTGATATCTACATCTGGGCGCCCTTGGCCGGGACTTGTGGGGTCCTTCTCCTGTCAC |
| | | TGGTTATCACCCTTTACTGCAAACGGGGCAGAAAGAAACTCCTGTATATATTCAAACA |
| | | ACCATTTATGAGACCAGTACAAACTACTCAAGAAGAGGACGGCTGTAGCTGCCGATTT |
| | | CCAGAAGAAGAAGAAGGAGGATGTGAACTGAGAGTGAAGTTCAGCAGGAGCGCAGACG |
| | | CCCCCGCGTACCAGCAGGGCCAGAACCAGCTCTATAACGAGCTCAATCTAGGACGAAG |
| | | AGAGGAGTACGATGTTTTGGACAAGAGGCGTGGCCGGGACCCTGAGATGGGGGGAAAG |
| | | CCGAGAAGGAAGAACCCTCAGGAAGGCCTGTACAATGAACTGCAGAAAGATAAGATGG |
| | | CGGAGGCCTACAGTGAGATTGGGATGAAAGGCGAGCGCCGGAGGGGCAAGGGGCACGA |
| | | TGGCCTTTACCAGGGTCTCAGTACAGCCACCAAGGACACCTACGACGCCCTTCACATG |
| | | CAGGCCCTGCCCCCTAGG |
| 44 | PSCA scFv | EIVLTQSPDFQSVTPTEKVTITCRASQYIGDSLHWYQQKPDQSPKLLIKYASQSFSGV |
| | AA | PSRFSGSGSGTDFTLTINSLEAEDAATYYCHQSSSLPYTFGQGTKLEIKGSTSGSGKP |
| | | GSGEGSTKGQVQLVESGGGVVQPGGSLRLSCAASGFTFSSYGMHWVRQAPGKGLEWVA |
| | | AIWFDGINKFYVDSVKGRFTISRDNSKNTLYLQMNSLRAEDTALYYCARKVHLERRRD |
| | | VYYYYMDVWGKGTTVTVSS |
| 45 | PSCA VH | QVQLVESGGGVVQPGGSLRLSCAASGFTFSSYGMHWVRQAPGKGLEWVAAIWEDGINK |
| | AA | FYVDSVKGRFTISRDNSKNTLYLQMNSLRAEDTALYYCARKVHLERRRDVYYYYMDVW |
| | | GKGTTVTVSS |
| 46 | PSCA VL | EIVLTQSPDFQSVTPTEKVTITCRASQYIGDSLHWYQQKPDQSPKLLIKYASQSFSGV |
| | AA | PSRFSGSGSGTDFTLTINSLEAEDAATYYCHQSSSLPYTFGQGTKLEIK |
| 47 | PSCA CDR | SYGMH |
| | H1 | |
| 48 | PSCA CDR | AIWFDGINKFYVDSVKG |
| | H2 | |
| 49 | PSCA CDR | KVHLERRRDVYYYYMDV |
| | H3 | |
| 50 | PSCA CDR | RASQYIGDSLH |
| | L1 | |
| 51 | PSCA CDR | YASQSFS |
| | L2 | |
| 52 | PSCA CDR | HQSSSLPYT |
| | L3 | |
| 53 | PSCA scFv | GAGATCGTGCTGACCCAGTCTCCAGACTTTCAGAGCGTGACACCCACCGAGAAAGTGA |
| | nucleic acid | CCATCACCTGTAGGGCCTCTCAGTACATCGGCGACAGCCTGCATTGGTACCAGCAGAA |
| | | GCCCGACCAGAGCCCTAAGCTGCTCATCAAATACGCCAGTCAGTCCTTCAGCGGCGTG |
| | | CCATCCAGATTCAGCGGCAGCGGCAGCGGAACCGACTTCACCCTGACCATCAACAGCC |
| | | TGGAGGCCGAGGACGCCGCCACCTACTATTGCCATCAGTCCTCCAGCCTGCCCTACAC |
| | | CTTCGGCCAGGGAACCAAGCTGGAAATCAAGGGCTCCACCAGCGGCAGCGGCAAGCCC |
| | | GGCAGCGGAGAAGGATCTACCAAGGGACAGGTCCAACTGGTGGAGAGCGGCGGCGGCG |
| | | TGGTGCAACCTGGAGGCTCCTTGAGGCTGAGCTGTGCTGCCAGCGGCTTCACCTTTTC |
| | | TAGCTACGGAATGCATTGGGTGCGGCAGGCTCCTGGCAAAGGCCTGGAGTGGGTGGCC |
| | | GCTATCTGGTTCGACGGCATCAACAAGTTCTACGTGGACAGCGTGAAGGGAAGGTTCA |
| | | CCATCAGCCGGGACAACAGCAAGAACACCCTGTACCTCCAGATGAACAGCCTGAGGGC |
| | | CGAGGATACCGCCCTGTACTATTGCGCCAGGAAAGTCCACCTGGAGAGGAGGAGGGAC |
| | | GTGTACTACTACTACATGGACGTGTGGGGCAAGGGCACCACCGTGACCGTGTCCAGC |
| 54 | PSCA | EIVLTQSPDFQSVTPTEKVTITCRASQYIGDSLHWYQQKPDQSPKLLIKYASQSFSGV |
| | primeR | PSRFSGSGSGTDFTLTINSLEAEDAATYYCHQSSSLPYTFGQGTKLEIKGSTSGSGKP |
| | protein full | GSGEGSTKGQVQLVESGGGVVQPGGSLRLSCAASGFTFSSYGMHWVRQAPGKGLEWVA |
| | sequence | AIWFDGINKFYVDSVKGRFTISRDNSKNTLYLQMNSLRAEDTALYYCARKVHLERRRD |
| | | VYYYYMDVWGKGTTVTVSSATTTPAPRPPTPAPTIASQPLSLRPEACEMYVAAAAFVL |
| | | LFFVGCGVLLSRKRRRQHGQLWFPEGFKVSEASKKKRREPLGEDSVGLKPLKNAMVSK |
| | | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVE |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDESSIADMDE |
| | | SALLSQISS |
| 55 | PSCA | GAGATCGTGCTGACCCAGTCTCCAGACTTTCAGAGCGTGACACCCACCGAGAAAGTGA |
| | primeR | CCATCACCTGTAGGGCCTCTCAGTACATCGGCGACAGCCTGCATTGGTACCAGCAGAA |
| | nucleic acid | GCCCGACCAGAGCCCTAAGCTGCTCATCAAATACGCCAGTCAGTCCTTCAGCGGCGTG |
| | full | CCATCCAGATTCAGCGGCAGCGGCAGCGGAACCGACTTCACCCTGACCATCAACAGCC |
| | sequence | TGGAGGCCGAGGACGCCGCCACCTACTATTGCCATCAGTCCTCCAGCCTGCCCTACAC |
| | | CTTCGGCCAGGGAACCAAGCTGGAAATCAAGGGCTCCACCAGCGGCAGCGGCAAGCCC |
| | | GGCAGCGGAGAAGGATCTACCAAGGGACAGGTCCAACTGGTGGAGAGCGGCGGCGGCG |
| | | TGGTGCAACCTGGAGGCTCCTTGAGGCTGAGCTGTGCTGCCAGCGGCTTCACCTTTTC |
| | | TAGCTACGGAATGCATTGGGTGCGGCAGGCTCCTGGCAAAGGCCTGGAGTGGGTGGCC |
| | | GCTATCTGGTTCGACGGCATCAACAAGTTCTACGTGGACAGCGTGAAGGGAAGGTTCA |
| | | CCATCAGCCGGGACAACAGCAAGAACACCCTGTACCTCCAGATGAACAGCCTGAGGGC |
| | | CGAGGATACCGCCCTGTACTATTGCGCCAGGAAAGTCCACCTGGAGAGGAGGAGGGAC |
| | | GTGTACTACTACTACATGGACGTGTGGGGCAAGGGCACCACCGTGACCGTGTCCAGCg |
| | | ccACCACAACACCCGCTCCCAGACCTCCCACACCAGCACCAACAATAGCAAGTCAACC |
| | | CTTGAGCTTGCGCCCTGAGGCCTGCTTCATGTACGTGGCCGCCGCCGCCTTCGTGTTG |
| | | CTGTTCTTCGTGGGCTGTGGCGTGCTGTTGTCCAGAAAGCGCCGCCGCCAGCACGGAC |
| | | AGCTCTGGTTCCCTGAGGGCTTCAAAGTGTCAGAGGCCAGCAAGAAGAAGCGGCGCGA |
| | | ACCTCTGGGCGAGGACAGCGTGGGCCTGAAGCCTCTGAAAAACGCTATGGTGTCAAAG |
| | | CTCAGCCAGCTCCAGACAGAGCTGCTGGCCGCCCTGCTGGAAAGCGGCCTGAGCAAGG |
| | | AGGCCCTGTTGCAGGCTCTGGGAGAGCCAGGCCCTTACCTGCTGGCTGGCGAGGGACC |
| | | TTTGGATAAGGGCGAGAGCTGCGGAGGCGGACGCGGAGAGCTGGCCGAACTGCCTAAT |
| | | GGCTTGGGCGAGACACGCGGCAGCGAGGACGAGACAGACGACGACGGCGAGGATTTCA |
| | | CACCTCCCATCCTCAAGGAGTTGGAGAATCTGTCTCCCGAGGAGGCCGCCCACCAGAA |
| | | GGCCGTGGTGGAGACACTGCTGCAAGAGGACCCTTGGCGCGTGGCCAAGATGGTGAAg |
| | | AGCTACCTGCAACAGCACAACATCCCTCAGCGCGAGGTGGTGGATACCACCGGCCTGA |
| | | ACCAGAGCCACCTGTCCCAGCACCTGAACAAGGGCACACCCATGAAGACCCAGAAGCG |
| | | CGCCGCCCTCTACACCTGGTACGTGCGCAAGCAGCGCGAGGTTGCTCAACAGTTCACC |
| | | CACGCCGGCCAGGGCGGCTTGATTGAGGAACCCACAGGCGACGAGCTGCCCACCAAGA |
| | | AGGGCCGCCGCAACCGCTTCAAATGGGGACCCGCCTCGCAGCAGATACTGTTCCAAGC |
| | | CTACGAGCGCCAGAAGAATCCCAGCAAGGAGGAGCGCGAGACACTCGTGGAGGAGTGC |
| | | AACCGCGCTGAGTGCATCCAGAGGGGCGTGAGCCCTAGCCAAGCCCAGGGCTTGGGCA |
| | | GCAACCTGGTGACAGAAGTGCGCGTGTATAACTGGTTCGCCAACCGCCGCAAGGAGGA |
| | | GGCCTTCCGCCACAAGCTGGCCATGACTTGCCGCGACGAGTTCCCTACAATGGTCTTT |
| | | CCCAGCGGCCAGATCAGCCAGGCCTCTGCCCTGGCACCAGCACCACCACAAGTGTTAC |
| | | CTCAAGCTCCCGCTCCCGCTCCCGCTCCTGCTATGGTGTCGGCATTGGCTCAAGCTCC |
| | | TGCTCCCGTGCCGGTGTTGGCACCTGGACCGCCTCAAGCTGTTGCTCCTCCCGCTCCC |
| | | AAGCCCACACAAGCCGGCGAAGGCACCCTGTCTGAGGCCCTGCTGCAACTGCAATTCG |
| | | ACGACGAGGATCTGGGCGCCCTGCTGGGCAACAGCACCGATCCCGCTGTCTTTACCGA |
| | | TCTGGCCAGCGTGGATAATAGCGAGTTCCAGCAGCTCCTGAACCAGGGTATCCCTGTG |
| | | GCGCCGCACACAACGGAGCCCATGCTGATGGAGTACCCTGAGGCCATCACCAGACTCG |
| | | TGACTGGCGCACAACGCCCTCCTGATCCAGCACCAGCGCCGTTAGGCGCACCTGGCCT |
| | | GCCTAACGGCTTGCTGAGCGGCGACGAGGATTTCTCCAGCATCGCCGATATGGATTTC |
| | | AGCGCACTGCTGAGTCAAATCTCCAGC |
| 56 | SPA full | ELPTQGTFSNVSTNVSELCGGAIVVPVCLAFLLTTLLGVLFCENKRDLIKKHIWPNVP |
| | protein | DPSKSHIAQWSPHTPPRHNENSKDQMYSDGNFTDVSVVEIEANDKKPFPEDLKSLDLF |
| | sequence | KKEKINTEGHSSGIGGSSCMSSSRPSISSSDENESSQNTSSTVQYSTVVHSGYRHQVP |
| | | SVQVFSRSESTQPLLDSEERPEDLQLVDHVDGGDGILPRQQYFKQNCSQHESSPDISH |
| | | FERSKQVSSVNEEDFVRLKQQISDHISQSCGSGQMKMFQEVSAADAFGPGTEGQVERF |
| | | ETVGMEAATDEGMPKSYLPQTVRQGGYMPQ |
| 57 | SPA full | GAGCTGCCCACACAAGGCACATTCAGCAACGTGTCCACCAACGTGTCCGAGCTGTGCG |
| | nucleic acid | GCGGCGCTATCGTGGTGCCCGTGTGCTTGGCCTTCCTGCTGACCACCCTGCTGGGCGT |
| | sequence | GCTGTTCTGCTTCAACAAGCGGGACCTGATTAAGAAGCACATCTGGCCCAACGTGCCC |
| | | GACCCTAGCAAGAGCCACATCGCCCAGTGGAGCCCTCACACACCTCCCAGGCACAACT |
| | | TCAACAGCAAGGACCAGATGTACTCCGACGGCAACTTCACCGACGTGTCCGTGGTGGA |
| | | GATCGAGGCCAACGACAAGAAGCCCTTCCCTGAGGACCTGAAGTCCCTGGACCTGTTC |
| | | AAGAAGGAGAAGATCAACACCGAGGGACACAGCAGCGGCATCGGCGGCTCTAGCTGTA |
| | | TGAGCAGCAGCAGGCCCAGCATCTCCAGCAGCGACGAGAACGAGAGCAGCCAGAACAC |
| | | CAGCAGCACCGTGCAGTACAGCACCGTGGTGCACAGCGGCTACAGGCACCAGGTGCCA |
| | | TCAGTGCAAGTGTTCAGCCGATCCGAGTCCACCCAGCCTCTGCTGGACAGCGAGGAGA |
| | | GGCCCGAGGACCTGCAACTGGTGGACCACGTGGACGGAGGCGACGGCATCCTGCCTAG |
| | | GCAGCAATACTTCAAGCAGAATTGCAGCCAGCACGAGAGCAGCCCTGACATCAGCCAC |
| | | TTCGAGAGGAGCAAGCAAGTGTCCAGCGTGAACGAGGAGGACTTCGTGAGGCTGAAGC |
| | | AGCAGATCAGCGACCACATCAGCCAGAGCTGCGGCAGCGGCCAGATGAAGATGTTCCA |
| | | GGAAGTGTCCGCCGCCGACGCCTTCGGCCCTGGCACAGAAGGACAAGTGGAGAGGTTC |
| | | GAAACCGTGGGCATGGAGGCCGCCACCGACGAGGGAATGCCCAAGAGCTACCTGCCTC |
| | | AGACCGTGCGGCAGGGCGGCTACATGCCTCAG |
| 58 | Logic gate 1 | GAGCCATGCTTGGCTTACGAGGGCGACCAACCCATCAAACTCCCCGCCCCCAGCACTT |
| | full insert | TTATTTCTCCTCTTTAGGAAGTACACTTCAGTATCTTTGGCACAGTGCATGAGCACGA |
| | | CTAAAGTAAAACATCGCAGAAAACATAGCTTTAGTCTACCCTTCGTGTCCTAAAAGGA |
| | | AAACCAGTAGCTTCCCAGGCCACCGGAAGGGCAACACATGTCCTCTGCAGTTTCTGCA |
| | | CACGGGAAGGTAAAGACAGAGAGAGGACCTACTCCTCAACACAGAAACATTTCAAAAT |
| | | CTTTCCTCGCCTGCAACCCAAGCTGAAGTCATTCTCCCCAGAAATAACAAAAGTTGGA |
| | | AGAGAAGCCGGAGACAGGATAGGTGCAGGAAGCCCACACTTTGAGGGCAGCACTCAGA |
| | | CACCCTCTCCTGTGTGCAGGACGTGCCGAATGTTCAGGTGCAATGAGAATGAGCCATG |
| | | CTTGGCTTATAaGGTAcgactgtgccttctagttgccagccatctgttgtttgcccct |
| | | cccccgtgccttccttgaccctggaaggtgccactcccactgtcctttcctaataaaa |
| | | tgaggaaattgcatcgcattgtctgagtaggtgtcattctattctggggggtggggtg |
| | | gggcaggacagcaagggggaggattgggaagacaatagcaggcatgctggggatgcgg |
| | | tgggctctatggGATAAGCTTGATAtcgaattcatcgatgttaataattaacatatat |
| | | gttaatcattaacatatagttaattattaaccgctatgttaatgattaacaacggtta |
| | | ataattaacatatatgttaatcattaacatataactagtctagagggtatataatggg |
| | | ggccactagtctactaccagagTtcatcgctagcgctaccggatccgccaccATGGCC |
| | | CTGCCAGTAACGGCTCTGCTGCTGCCACTTGCTCTGCTCCTCCATGCAGCCAGGCCTC |
| | | AGGTTCAGCTTGTGGAATCTGGTGGTGGCGTGGTTCAGCCTGGGAGATCTCTGCGGCT |
| | | GTCATGCGCTGCGTCTGGGTTCATCTTCTCCTCATACGGAATGCATTGGGTCAGACAG |
| | | GCTCCCGGCAAAGGGCTCGAGTGGGTTACAATTATCTGGTATGATGGCAGTAATCGCT |
| | | ACTACGCCGATTCCGTCAAAGGGCGGTTCACCATTTCAAGGGACAATAGCAAGAACAC |
| | | TTTGTATCTCCAAATGAACTCCCTTAGAGCAGAGGACACCGCCGTGTACTATTGCGCC |
| | | CGCGCAAGCGGCGACTTCTACAATTATTTTATGGACGTCTGGGGCAAGGGCACTACGG |
| | | TGACAGTGTCCTCCGGAGGAGGAGGATCTGGAGGAGGAGGAAGCGGTGGCGGCGGGAG |
| | | TGAGATAGTAATGACCCAGTCGCCTGCCACTCTAAGCGTATCTCCCGGCGAACGGGCT |
| | | ACCTTGAGTTGTAGGGCAAGTCAATCAGTGTCCTCCAATCTGGCCTGGTATCAGCAAA |
| | | AACCGGGCCAGGCTCCTAGGCTGCTGATATACGGCGCCAGCACACGTGCAACTGGGAT |
| | | TCCAGCGCGATTTAGCGGGAGCGGTTCAGGGACGGAGTTCACCTTAACAATCAGTAGC |
| | | CTGCAATCTGAGGATTTTGCCGTGTACTATTGTCAACAGTATAACAACTGGCCACCCT |
| | | ACACCTTTGGCCAGGGTACAAAGCTCGAAATCAAGGCGGcagcaaccacgacGCCAGC |
| | | GCCGCGACCACCAACACCGGCGCCCACCATCGCGTCGCAGCCACTGTCACTGCGCCCA |
| | | GAAGCGTGCCGGCCAGCGGCGGGGGGCGCAGTGCACACGAGGGGGCTGGACTTCGCCT |
| | | GTGATATCTACATCTGGGCGCCCTTGGCCGGGACTTGTGGGGTCCTTCTCCTGTCACT |
| | | GGTTATCACCCTTTACTGCAAACGGGGCAGAAAGAAACTCCTGTATATATTCAAACAA |
| | | CCATTTATGAGACCAGTACAAACTACTCAAGAAGAGGACGGCTGTAGCTGCCGATTTC |
| | | CAGAAGAAGAAGAAGGAGGATGTGAACTGAGAGTGAAGTTCAGCAGGAGCGCAGACGC |
| | | CCCCGCGTACCAGCAGGGCCAGAACCAGCTCTATAACGAGCTCAATCTAGGACGAAGA |
| | | GAGGAGTACGATGTTTTGGACAAGAGGCGTGGCCGGGACCCTGAGATGGGGGGAAAGC |
| | | CGAGAAGGAAGAACCCTCAGGAAGGCCTGTACAATGAACTGCAGAAAGATAAGATGGC |
| | | GGAGGCCTACAGTGAGATTGGGATGAAAGGCGAGCGCCGGAGGGGCAAGGGGCACGAT |
| | | GGCCTTTACCAGGGTCTCAGTACAGCCACCAAGGACACCTACGACGCCCTTCACATGC |
| | | AGGCCCTGCCCCCTAGGTAAAATCAACCTCTGGATTACAAAATTTGTGAAAGATTGAC |
| | | TGGTATTCTTAACTATGTTGCTCCTTTTACGCTATGTGGATACGCTGCTTTAATGCCT |
| | | TTGTATCATGCTATTGCTTCCCGTATGGCTTTCATTTTCTCCTCCTTGTATAAATCCT |
| | | GGTTGCTGTCTCTTTATGAGGAGTTGTGGCCCGTTGTCAGGCAACGTGGCGTGGTGTG |
| | | CACTGTGTTTGCTGACGCAACCCCCACTGGTTGGGGCATTGCCACCACCTGTCAGCTC |
| | | CTTTCCGGGACTTTCGCTTTCCCCCTCCCTATTGCCACGGCGGAACTCATCGCCGCCT |
| | | GCCTTGCCCGCTGCTGGACAGGGGCTCGGCTGTTGGGCACTGACAATTCCGTGGTGTT |
| | | GTCGGGGAAATCATCGTCCTTTCCTTGGCTGCTCGCCTGTGTTGCCACCTGGATTCTG |
| | | CGCGGGACGTCCTTCTGCTACGTCCCTTCGGCCCTCAATCCAGCGGACCTTCCTTCCC |
| | | GCGGCCTGCTGCCGGCTCTGCGGCCTCTTCCGCGTCTTCGCCTTCGCCCTCAGACGAG |
| | | TCGGATCTCCCTTTGGGCCGCCTCCCCGCCTGGATCCTTGACTTGCGGCCAACTTGTT |
| | | TATTGCAGCTTATAATGGTTACAAATAAAGCAATAGCATCACAAATTTCACAAATAAA |
| | | GCATTTTTTTCACTGCATTCTAGTTGTGGTTTGTCCAAACTCATCAATGTATCTTATC |
| | | ATGTCTGGGATCCTTGACTTGCGGCCGCAACTCCCACCTGCAACATGCGTGACTGACT |
| | | GAGGCCGCGACTCTAGAGTCGACCGGATCTGCGATCGCTCCGGTGCCCGTCAGTGGGC |
| | | AGAGCGCACATCGCCCACAGTCCCCGAGAAGTTGGGGGGAGGGGTCGGCAATTGAACG |
| | | GGTGCCTAGAGAAGGTGGCGCGGGGTAAACTGGGAAAGTGATGTCGTGTACTGGCTCC |
| | | GCCTTTTTCCCGAGGGTGGGGGAGAACCGTATATAAGTGCAGTAGTCGCCGTGAACGT |
| | | TCTTTTTCGCAACGGGTTTGCCGCCAGAACACAGCTGAAGCTTCGAGGGGCTCGCATC |
| | | TCTCCTTCACGCGCCCGCCGCCCTACCTGAGGCCGCCATCCACGCCGGTTGAGTCGCG |
| | | TTCTGCCGCCTCCCGCCTGTGGTGCCTCCtgaactgcgtccgccgtctaggtaaGTcg |
| | | actcgttggatccCCACTACCCGGATCAACGCCCTAGGTTTATGTTTGGATGAACTGA |
| | | CATACGCGTATCCGTCTTAAGAATCTTTTCAAACACTAGTAGTGAAATATATATTAAA |
| | | CTAGTGTTTGAAAAGATTCTTATTACGGTAACGCGGAATTCGCAACTATTTTATCAAT |
| | | TTTTTGCGTCGACACTTCAAGGGGCTTGCGGCCGCAACCATCTCCATGGCTGTTTGAA |
| | | TGAGGCTTCAGTACTTTACAGAATCGTTGCCTGCACATCTTGGAAACACTTGCTGGGA |
| | | TTACTTCGACTTCTTAACCCAACAGAAGGCTCGAGAAGGTATATTGCTGTTGACAGTG |
| | | AGCGCCATAGGAAACTTGTTAATCATTAGTGAAGCCACAGATGTATTGATTAACAAGT |
| | | TTCCTATGGTGCCTACTGCCTCGGACTTCAAGGGGCTAGAATTCGAGCAATTATCTTG |
| | | TTTACTAAAACTGAATACCTTGCTATCTCTTTGATACATTTTTACAAAGCTGAATTAA |
| | | AATGGTATAAATTAAATCACTTTGACGGTGACTTAGGAGTATGCCGGATCAACGCCCT |
| | | AGGTTTATGTTTGGATGAACTGACATACGCGTATCCGTCTAAAAATCAATCTCATTTC |
| | | CTGGTAGTGAAATATATATTAAACCAGGAAATGAGATTGATTTTTTTACGGTAACGCG |
| | | GAATTCGCAACTATTTTATCAATTTTTTGCGTCGACGACTGTGACAGCAGAGTATGCC |
| | | GGATCAACGCCCTAGGTTTATGTTTGGATGAACTGACATACGCGTATCCGTCTTATGT |
| | | AAAAGACAAACAATGCGTAGTGAAATATATATTAAACGCATTGTTTGTCTTTTACATA |
| | | TTACGGTAACGCGGAATTCGCAACTATTTTATCAATTTTTTGCGTCGACCGGAACTAT |
| | | CTTGAAGAGTAGTAGTGGactagtgtgacgctgctgacccctttctttcccttctACA |
| | | GATCCAAGCTGTGACCGGCGCCTACacctgcagcccaagcttaccatggccttaccag |
| | | tgaccgccttgctcctgccgctggccttgctgctccacgccgccaggcctGAGATCGT |
| | | GCTGACCCAGTCTCCAGACTTTCAGAGCGTGACACCCACCGAGAAAGTGACCATCACC |
| | | TGTAGGGCCTCTCAGTACATCGGCGACAGCCTGCATTGGTACCAGCAGAAGCCCGACC |
| | | AGAGCCCTAAGCTGCTCATCAAATACGCCAGTCAGTCCTTCAGCGGCGTGCCATCCAG |
| | | ATTCAGCGGCAGCGGCAGCGGAACCGACTTCACCCTGACCATCAACAGCCTGGAGGCC |
| | | GAGGACGCCGCCACCTACTATTGCCATCAGTCCTCCAGCCTGCCCTACACCTTCGGCC |
| | | AGGGAACCAAGCTGGAAATCAAGGGCTCCACCAGCGGCAGCGGCAAGCCCGGCAGCGG |
| | | AGAAGGATCTACCAAGGGACAGGTCCAACTGGTGGAGAGCGGCGGCGGCGTGGTGCAA |
| | | CCTGGAGGCTCCTTGAGGCTGAGCTGTGCTGCCAGCGGCTTCACCTTTTCTAGCTACG |
| | | GAATGCATTGGGTGCGGCAGGCTCCTGGCAAAGGCCTGGAGTGGGTGGCCGCTATCTG |
| | | GTTCGACGGCATCAACAAGTTCTACGTGGACAGCGTGAAGGGAAGGTTCACCATCAGC |
| | | CGGGACAACAGCAAGAACACCCTGTACCTCCAGATGAACAGCCTGAGGGCCGAGGATA |
| | | CCGCCCTGTACTATTGCGCCAGGAAAGTCCACCTGGAGAGGAGGAGGGACGTGTACTA |
| | | CTACTACATGGACGTGTGGGGCAAGGGCACCACCGTGACCGTGTCCAGCgccACCACA |
| | | ACACCCGCTCCCAGACCTCCCACACCAGCACCAACAATAGCAAGTCAACCCTTGAGCT |
| | | TGCGCCCTGAGGCCTGCTTCATGTACGTGGCCGCCGCCGCCTTCGTGTTGCTGTTCTT |
| | | CGTGGGCTGTGGCGTGCTGTTGTCCAGAAAGCGCCGCCGCCAGCACGGACAGCTCTGG |
| | | TTCCCTGAGGGCTTCAAAGTGTCAGAGGCCAGCAAGAAGAAGCGGCGCGAACCTCTGG |
| | | GCGAGGACAGCGTGGGCCTGAAGCCTCTGAAAAACGCTATGGTGTCAAAGCTCAGCCA |
| | | GCTCCAGACAGAGCTGCTGGCCGCCCTGCTGGAAAGCGGCCTGAGCAAGGAGGCCCTG |
| | | TTGCAGGCTCTGGGAGAGCCAGGCCCTTACCTGCTGGCTGGCGAGGGACCTTTGGATA |
| | | AGGGCGAGAGCTGCGGAGGCGGACGCGGAGAGCTGGCCGAACTGCCTAATGGCTTGGG |
| | | CGAGACACGCGGCAGCGAGGACGAGACAGACGACGACGGCGAGGATTTCACACCTCCC |
| | | ATCCTCAAGGAGTTGGAGAATCTGTCTCCCGAGGAGGCCGCCCACCAGAAGGCCGTGG |
| | | TGGAGACACTGCTGCAAGAGGACCCTTGGCGCGTGGCCAAGATGGTGAAgAGCTACCT |
| | | GCAACAGCACAACATCCCTCAGCGCGAGGTGGTGGATACCACCGGCCTGAACCAGAGC |
| | | CACCTGTCCCAGCACCTGAACAAGGGCACACCCATGAAGACCCAGAAGCGCGCCGCCC |
| | | TCTACACCTGGTACGTGCGCAAGCAGCGCGAGGTTGCTCAACAGTTCACCCACGCCGG |
| | | CCAGGGCGGCTTGATTGAGGAACCCACAGGCGACGAGCTGCCCACCAAGAAGGGCCGC |
| | | CGCAACCGCTTCAAATGGGGACCCGCCTCGCAGCAGATACTGTTCCAAGCCTACGAGC |
| | | GCCAGAAGAATCCCAGCAAGGAGGAGCGCGAGACACTCGTGGAGGAGTGCAACCGCGC |
| | | TGAGTGCATCCAGAGGGGCGTGAGCCCTAGCCAAGCCCAGGGCTTGGGCAGCAACCTG |
| | | GTGACAGAAGTGCGCGTGTATAACTGGTTCGCCAACCGCCGCAAGGAGGAGGCCTTCC |
| | | GCCACAAGCTGGCCATGACTTGCCGCGACGAGTTCCCTACAATGGTCTTTCCCAGCGG |
| | | CCAGATCAGCCAGGCCTCTGCCCTGGCACCAGCACCACCACAAGTGTTACCTCAAGCT |
| | | CCCGCTCCCGCTCCCGCTCCTGCTATGGTGTCGGCATTGGCTCAAGCTCCTGCTCCCG |
| | | TGCCGGTGTTGGCACCTGGACCGCCTCAAGCTGTTGCTCCTCCCGCTCCCAAGCCCAC |
| | | ACAAGCCGGCGAAGGCACCCTGTCTGAGGCCCTGCTGCAACTGCAATTCGACGACGAG |
| | | GATCTGGGCGCCCTGCTGGGCAACAGCACCGATCCCGCTGTCTTTACCGATCTGGCCA |
| | | GCGTGGATAATAGCGAGTTCCAGCAGCTCCTGAACCAGGGTATCCCTGTGGCGCCGCA |
| | | CACAACGGAGCCCATGCTGATGGAGTACCCTGAGGCCATCACCAGACTCGTGACTGGC |
| | | GCACAACGCCCTCCTGATCCAGCACCAGCGCCGTTAGGCGCACCTGGCCTGCCTAACG |
| | | GCTTGCTGAGCGGCGACGAGGATTTCTCCAGCATCGCCGATATGGATTTCAGCGCACT |
| | | GCTGAGTCAAATCTCCAGCCGGGCCAAGCGGTCCGGATCCGGAGAGGGCAGGGGATCT |
| | | CTCCTTACTTGTGGGGACGTCGAGGAAAACCCTGGACCaATGGCCCTGCCCGTGACAG |
| | | CCCTGCTCCTGCCTCTGGCCCTGTTGCTGCATGCAGCACGGCCTGAGCTGCCCACACA |
| | | AGGCACATTCAGCAACGTGTCCACCAACGTGTCCGAGCTGTGCGGCGGCGCTATCGTG |
| | | GTGCCCGTGTGCTTGGCCTTCCTGCTGACCACCCTGCTGGGCGTGCTGTTCTGCTTCA |
| | | ACAAGCGGGACCTGATTAAGAAGCACATCTGGCCCAACGTGCCCGACCCTAGCAAGAG |
| | | CCACATCGCCCAGTGGAGCCCTCACACACCTCCCAGGCACAACTTCAACAGCAAGGAC |
| | | CAGATGTACTCCGACGGCAACTTCACCGACGTGTCCGTGGTGGAGATCGAGGCCAACG |
| | | ACAAGAAGCCCTTCCCTGAGGACCTGAAGTCCCTGGACCTGTTCAAGAAGGAGAAGAT |
| | | CAACACCGAGGGACACAGCAGCGGCATCGGCGGCTCTAGCTGTATGAGCAGCAGCAGG |
| | | CCCAGCATCTCCAGCAGCGACGAGAACGAGAGCAGCCAGAACACCAGCAGCACCGTGC |
| | | AGTACAGCACCGTGGTGCACAGCGGCTACAGGCACCAGGTGCCATCAGTGCAAGTGTT |
| | | CAGCCGATCCGAGTCCACCCAGCCTCTGCTGGACAGCGAGGAGAGGCCCGAGGACCTG |
| | | CAACTGGTGGACCACGTGGACGGAGGCGACGGCATCCTGCCTAGGCAGCAATACTTCA |
| | | AGCAGAATTGCAGCCAGCACGAGAGCAGCCCTGACATCAGCCACTTCGAGAGGAGCAA |
| | | GCAAGTGTCCAGCGTGAACGAGGAGGACTTCGTGAGGCTGAAGCAGCAGATCAGCGAC |
| | | CACATCAGCCAGAGCTGCGGCAGCGGCCAGATGAAGATGTTCCAGGAAGTGTCCGCCG |
| | | CCGACGCCTTCGGCCCTGGCACAGAAGGACAAGTGGAGAGGTTCGAAACCGTGGGCAT |
| | | GGAGGCCGCCACCGACGAGGGAATGCCCAAGAGCTACCTGCCTCAGACCGTGCGGCAG |
| | | GGCGGCTACATGCCTCAGTAAAGGACGGGTGGCATCCCTGTGACCCCTCCCCAGTGCC |
| | | TCTCCTGGCCCTGGAAGTTGCCACTCCAGTGCCCACCAGCCTTGTCCTAATAAAATTA |
| | | AGTTGCATCATTTTGTCTGACTAGGTGTCCTTCTATAATATTATGGGGTGGAGGGGGG |
| | | TGGTATGGAGCAAGGGGCAAGTTGGGAAGACAACCTGTAGGGCCTGCGGGGTCTATTG |
| | | GGAACCAAGCTGGAGTGCAGTGGCACAATCTTGGCTCACTGCAATCTCCGCCTCCTGG |
| | | GTTCAAGCGATTCTCCTGCCTCAGCCTCCCGAGTTGTTGGGATTCCAGGCATGCATGA |
| | | CCAGGCTCAGCTAATTTTTGTTTTTTTGGTAGAaACGGGGTTTCACCATATTGGCCAG |
| | | GCTGGTCTCCAACTCCTAATCTCAGGTGATCTACCCACCTTGGCCTCCCAAATTGCTG |
| | | GGATTACAGGCGTGAACCACTGCTCCCttccctgtccttcCGAGGGCAATCTGGCCCA |
| | | TCAAGTGGCCTTCGCCTCTGGGAGTAACAAAAATGCACTTCAAAATAGCTTCTGTAAT |
| | | CAAGCTGCATGGGTGGAGTACTCCCCAGCTGACTCCAGGAAGTTCTCTATCCAAAGCT |
| | | ATTCATTAGGCCAGAGCTGTGCAAATAATTAGTCACCCACTTGCTCCATAACCCTCCA |
| | | TGACAGCCCAGGCATTGAGTCCAGGTGGGACCATCAAGCCATGCTCTGGTGGCTCATG |
| | | CATTATCATAGAAATGGGAGGCTTTATTTATTTTACTAAAAAGAACAAAAACAACAGA |
| | | CTGCTGTCCTTTAGACAATAGGATCACGTCATCTGAGCCCTCTGTGCCCCAGGTGACA |
| | | AGCCCAGCCCCAAGTTCTCTTTCCTCAGCCTCCCCACACATGTTCTGGAGGAGATGGG |
| | | CCCAGCAGGCTGCTCTGAGGCCTGGCCCCTCGTAAGCCAAGCATGGCTC |
| 59 | Logic gate 1 | GGGCGACCAACCCATCAAACTCCCCGCCCCCAGCACTTTTATTTCTCCTCTTTAGGAA |
| | insert | GTACACTTCAGTATCTTTGGCACAGTGCATGAGCACGACTAAAGTAAAACATCGCAGA |
| | without | AAACATAGCTTTAGTCTACCCTTCGTGTCCTAAAAGGAAAACCAGTAGCTTCCCAGGC |
| | insertion | CACCGGAAGGGCAACACATGTCCTCTGCAGTTTCTGCACACGGGAAGGTAAAGACAGA |
| | sites | GAGAGGACCTACTCCTCAACACAGAAACATTTCAAAATCTTTCCTCGCCTGCAACCCA |
| | | AGCTGAAGTCATTCTCCCCAGAAATAACAAAAGTTGGAAGAGAAGCCGGAGACAGGAT |
| | | AGGTGCAGGAAGCCCACACTTTGAGGGCAGCACTCAGACACCCTCTCCTGTGTGCAGG |
| | | ACGTGCCGAATGTTCAGGTGCAATGAGAATGAGCCATGCTTGGCTTATAaGGTAcgac |
| | | tgtgccttctagttgccagccatctgttgtttgcccctcccccgtgccttccttgacc |
| | | ctggaaggtgccactcccactgtcctttcctaataaaatgaggaaattgcatcgcatt |
| | | gtctgagtaggtgtcattctattctggggggtggggggggcaggacagcaaggggga |
| | | ggattgggaagacaatagcaggcatgctggggatgcggtgggctctatggGATAAGCT |
| | | TGATAtcgaattcatcgatgttaataattaacatatatgttaatcattaacatatagt |
| | | taattattaaccgctatgttaatgattaacaacggttaataattaacatatatgttaa |
| | | tcattaacatataactagtctagagggtatataatgggggccactagtctactaccag |
| | | agTtcatcgctagcgctaccggatccgccaccATGGCCCTGCCAGTAACGGCTCTGCT |
| | | GCTGCCACTTGCTCTGCTCCTCCATGCAGCCAGGCCTCAGGTTCAGCTTGTGGAATCT |
| | | GGTGGTGGCGTGGTTCAGCCTGGGAGATCTCTGCGGCTGTCATGCGCTGCGTCTGGGT |
| | | TCATCTTCTCCTCATACGGAATGCATTGGGTCAGACAGGCTCCCGGCAAAGGGCTCGA |
| | | GTGGGTTACAATTATCTGGTATGATGGCAGTAATCGCTACTACGCCGATTCCGTCAAA |
| | | GGGCGGTTCACCATTTCAAGGGACAATAGCAAGAACACTTTGTATCTCCAAATGAACT |
| | | CCCTTAGAGCAGAGGACACCGCCGTGTACTATTGCGCCCGCGCAAGCGGCGACTTCTA |
| | | CAATTATTTTATGGACGTCTGGGGCAAGGGCACTACGGTGACAGTGTCCTCCGGAGGA |
| | | GGAGGATCTGGAGGAGGAGGAAGCGGTGGCGGCGGGAGTGAGATAGTAATGACCCAGT |
| | | CGCCTGCCACTCTAAGCGTATCTCCCGGCGAACGGGCTACCTTGAGTTGTAGGGCAAG |
| | | TCAATCAGTGTCCTCCAATCTGGCCTGGTATCAGCAAAAACCGGGCCAGGCTCCTAGG |
| | | CTGCTGATATACGGCGCCAGCACACGTGCAACTGGGATTCCAGCGCGATTTAGCGGGA |
| | | GCGGTTCAGGGACGGAGTTCACCTTAACAATCAGTAGCCTGCAATCTGAGGATTTTGC |
| | | CGTGTACTATTGTCAACAGTATAACAACTGGCCACCCTACACCTTTGGCCAGGGTACA |
| | | AAGCTCGAAATCAAGGCGGcagcaaccacgacGCCAGCGCCGCGACCACCAACACCGG |
| | | CGCCCACCATCGCGTCGCAGCCACTGTCACTGCGCCCAGAAGCGTGCCGGCCAGCGGC |
| | | GGGGGGCGCAGTGCACACGAGGGGGCTGGACTTCGCCTGTGATATCTACATCTGGGCG |
| | | CCCTTGGCCGGGACTTGTGGGGTCCTTCTCCTGTCACTGGTTATCACCCTTTACTGCA |
| | | AACGGGGCAGAAAGAAACTCCTGTATATATTCAAACAACCATTTATGAGACCAGTACA |
| | | AACTACTCAAGAAGAGGACGGCTGTAGCTGCCGATTTCCAGAAGAAGAAGAAGGAGGA |
| | | TGTGAACTGAGAGTGAAGTTCAGCAGGAGCGCAGACGCCCCCGCGTACCAGCAGGGCC |
| | | AGAACCAGCTCTATAACGAGCTCAATCTAGGACGAAGAGAGGAGTACGATGTTTTGGA |
| | | CAAGAGGCGTGGCCGGGACCCTGAGATGGGGGGAAAGCCGAGAAGGAAGAACCCTCAG |
| | | GAAGGCCTGTACAATGAACTGCAGAAAGATAAGATGGCGGAGGCCTACAGTGAGATTG |
| | | GGATGAAAGGCGAGCGCCGGAGGGGCAAGGGGCACGATGGCCTTTACCAGGGTCTCAG |
| | | TACAGCCACCAAGGACACCTACGACGCCCTTCACATGCAGGCCCTGCCCCCTAGGTAA |
| | | AATCAACCTCTGGATTACAAAATTTGTGAAAGATTGACTGGTATTCTTAACTATGTTG |
| | | CTCCTTTTACGCTATGTGGATACGCTGCTTTAATGCCTTTGTATCATGCTATTGCTTC |
| | | CCGTATGGCTTTCATTTTCTCCTCCTTGTATAAATCCTGGTTGCTGTCTCTTTATGAG |
| | | GAGTTGTGGCCCGTTGTCAGGCAACGTGGCGTGGTGTGCACTGTGTTTGCTGACGCAA |
| | | CCCCCACTGGTTGGGGCATTGCCACCACCTGTCAGCTCCTTTCCGGGACTTTCGCTTT |
| | | CCCCCTCCCTATTGCCACGGCGGAACTCATCGCCGCCTGCCTTGCCCGCTGCTGGACA |
| | | GGGGCTCGGCTGTTGGGCACTGACAATTCCGTGGTGTTGTCGGGGAAATCATCGTCCT |
| | | TTCCTTGGCTGCTCGCCTGTGTTGCCACCTGGATTCTGCGCGGGACGTCCTTCTGCTA |
| | | CGTCCCTTCGGCCCTCAATCCAGCGGACCTTCCTTCCCGCGGCCTGCTGCCGGCTCTG |
| | | CGGCCTCTTCCGCGTCTTCGCCTTCGCCCTCAGACGAGTCGGATCTCCCTTTGGGCCG |
| | | CCTCCCCGCCTGGATCCTTGACTTGCGGCCAACTTGTTTATTGCAGCTTATAATGGTT |
| | | ACAAATAAAGCAATAGCATCACAAATTTCACAAATAAAGCATTTTTTTCACTGCATTC |
| | | TAGTTGTGGTTTGTCCAAACTCATCAATGTATCTTATCATGTCTGGGATCCTTGACTT |
| | | GCGGCCGCAACTCCCACCTGCAACATGCGTGACTGACTGAGGCCGCGACTCTAGAGTC |
| | | GACCGGATCTGCGATCGCTCCGGTGCCCGTCAGTGGGCAGAGCGCACATCGCCCACAG |
| | | TCCCCGAGAAGTTGGGGGGAGGGGTCGGCAATTGAACGGGTGCCTAGAGAAGGTGGCG |
| | | CGGGGTAAACTGGGAAAGTGATGTCGTGTACTGGCTCCGCCTTTTTCCCGAGGGTGGG |
| | | GGAGAACCGTATATAAGTGCAGTAGTCGCCGTGAACGTTCTTTTTCGCAACGGGTTTG |
| | | CCGCCAGAACACAGCTGAAGCTTCGAGGGGCTCGCATCTCTCCTTCACGCGCCCGCCG |
| | | CCCTACCTGAGGCCGCCATCCACGCCGGTTGAGTCGCGTTCTGCCGCCTCCCGCCTGT |
| | | GGTGCCTCCtgaactgcgtccgccgtctaggtaaGTcgactcgttggatccCCACTAC |
| | | CCGGATCAACGCCCTAGGTTTATGTTTGGATGAACTGACATACGCGTATCCGTCTTAA |
| | | GAATCTTTTCAAACACTAGTAGTGAAATATATATTAAACTAGTGTTTGAAAAGATTCT |
| | | TATTACGGTAACGCGGAATTCGCAACTATTTTATCAATTTTTTGCGTCGACACTTCAA |
| | | GGGGCTTGCGGCCGCAACCATCTCCATGGCTGTTTGAATGAGGCTTCAGTACTTTACA |
| | | GAATCGTTGCCTGCACATCTTGGAAACACTTGCTGGGATTACTTCGACTTCTTAACCC |
| | | AACAGAAGGCTCGAGAAGGTATATTGCTGTTGACAGTGAGCGCCATAGGAAACTTGTT |
| | | AATCATTAGTGAAGCCACAGATGTATTGATTAACAAGTTTCCTATGGTGCCTACTGCC |
| | | TCGGACTTCAAGGGGCTAGAATTCGAGCAATTATCTTGTTTACTAAAACTGAATACCT |
| | | TGCTATCTCTTTGATACATTTTTACAAAGCTGAATTAAAATGGTATAAATTAAATCAC |
| | | TTTGACGGTGACTTAGGAGTATGCCGGATCAACGCCCTAGGTTTATGTTTGGATGAAC |
| | | TGACATACGCGTATCCGTCTAAAAATCAATCTCATTTCCTGGTAGTGAAATATATATT |
| | | AAACCAGGAAATGAGATTGATTTTTTTACGGTAACGCGGAATTCGCAACTATTTTATC |
| | | AATTTTTTGCGTCGACGACTGTGACAGCAGAGTATGCCGGATCAACGCCCTAGGTTTA |
| | | TGTTTGGATGAACTGACATACGCGTATCCGTCTTATGTAAAAGACAAACAATGCGTAG |
| | | TGAAATATATATTAAACGCATTGTTTGTCTTTTACATATTACGGTAACGCGGAATTCG |
| | | CAACTATTTTATCAATTTTTTGCGTCGACCGGAACTATCTTGAAGAGTAGTAGTGGac |
| | | tagtgtgacgctgctgacccctttctttcccttctACAGATCCAAGCTGTGACCGGCG |
| | | CCTACacctgcagcccaagcttaccatggccttaccagtgaccgccttgctcctgccg |
| | | ctggccttgctgctccacgccgccaggcctGAGATCGTGCTGACCCAGTCTCCAGACT |
| | | TTCAGAGCGTGACACCCACCGAGAAAGTGACCATCACCTGTAGGGCCTCTCAGTACAT |
| | | CGGCGACAGCCTGCATTGGTACCAGCAGAAGCCCGACCAGAGCCCTAAGCTGCTCATC |
| | | AAATACGCCAGTCAGTCCTTCAGCGGCGTGCCATCCAGATTCAGCGGCAGCGGCAGCG |
| | | GAACCGACTTCACCCTGACCATCAACAGCCTGGAGGCCGAGGACGCCGCCACCTACTA |
| | | TTGCCATCAGTCCTCCAGCCTGCCCTACACCTTCGGCCAGGGAACCAAGCTGGAAATC |
| | | AAGGGCTCCACCAGCGGCAGCGGCAAGCCCGGCAGCGGAGAAGGATCTACCAAGGGAC |
| | | AGGTCCAACTGGTGGAGAGCGGCGGCGGCGTGGTGCAACCTGGAGGCTCCTTGAGGCT |
| | | GAGCTGTGCTGCCAGCGGCTTCACCTTTTCTAGCTACGGAATGCATTGGGTGCGGCAG |
| | | GCTCCTGGCAAAGGCCTGGAGTGGGTGGCCGCTATCTGGTTCGACGGCATCAACAAGT |
| | | TCTACGTGGACAGCGTGAAGGGAAGGTTCACCATCAGCCGGGACAACAGCAAGAACAC |
| | | CCTGTACCTCCAGATGAACAGCCTGAGGGCCGAGGATACCGCCCTGTACTATTGCGCC |
| | | AGGAAAGTCCACCTGGAGAGGAGGAGGGACGTGTACTACTACTACATGGACGTGTGGG |
| | | GCAAGGGCACCACCGTGACCGTGTCCAGCgccACCAcAACACCCGCTCCCAGACCTCC |
| | | CACACCAGCACCAACAATAGCAAGTCAACCCTTGAGCTTGCGCCCTGAGGCCTGCTTC |
| | | ATGTACGTGGCCGCCGCCGCCTTCGTGTTGCTGTTCTTCGTGGGCTGTGGCGTGCTGT |
| | | TGTCCAGAAAGCGCCGCCGCCAGCACGGACAGCTCTGGTTCCCTGAGGGCTTCAAAGT |
| | | GTCAGAGGCCAGCAAGAAGAAGCGGCGCGAACCTCTGGGCGAGGACAGCGTGGGCCTG |
| | | AAGCCTCTGAAAAACGCTATGGTGTCAAAGCTCAGCCAGCTCCAGACAGAGCTGCTGG |
| | | CCGCCCTGCTGGAAAGCGGCCTGAGCAAGGAGGCCCTGTTGCAGGCTCTGGGAGAGCC |
| | | AGGCCCTTACCTGCTGGCTGGCGAGGGACCTTTGGATAAGGGCGAGAGCTGCGGAGGC |
| | | GGACGCGGAGAGCTGGCCGAACTGCCTAATGGCTTGGGCGAGACACGCGGCAGCGAGG |
| | | ACGAGACAGACGACGACGGCGAGGATTTCACACCTCCCATCCTCAAGGAGTTGGAGAA |
| | | TCTGTCTCCCGAGGAGGCCGCCCACCAGAAGGCCGTGGTGGAGACACTGCTGCAAGAG |
| | | GACCCTTGGCGCGTGGCCAAGATGGTGAAgAGCTACCTGCAACAGCACAACATCCCTC |
| | | AGCGCGAGGTGGTGGATACCACCGGCCTGAACCAGAGCCACCTGTCCCAGCACCTGAA |
| | | CAAGGGCACACCCATGAAGACCCAGAAGCGCGCCGCCCTCTACACCTGGTACGTGCGC |
| | | AAGCAGCGCGAGGTTGCTCAACAGTTCACCCACGCCGGCCAGGGCGGCTTGATTGAGG |
| | | AACCCACAGGCGACGAGCTGCCCACCAAGAAGGGCCGCCGCAACCGCTTCAAATGGGG |
| | | ACCCGCCTCGCAGCAGATACTGTTCCAAGCCTACGAGCGCCAGAAGAATCCCAGCAAG |
| | | GAGGAGCGCGAGACACTCGTGGAGGAGTGCAACCGCGCTGAGTGCATCCAGAGGGGCG |
| | | TGAGCCCTAGCCAAGCCCAGGGCTTGGGCAGCAACCTGGTGACAGAAGTGCGCGTGTA |
| | | TAACTGGTTCGCCAACCGCCGCAAGGAGGAGGCCTTCCGCCACAAGCTGGCCATGACT |
| | | TGCCGCGACGAGTTCCCTACAATGGTCTTTCCCAGCGGCCAGATCAGCCAGGCCTCTG |
| | | CCCTGGCACCAGCACCACCACAAGTGTTACCTCAAGCTCCCGCTCCCGCTCCCGCTCC |
| | | TGCTATGGTGTCGGCATTGGCTCAAGCTCCTGCTCCCGTGCCGGTGTTGGCACCTGGA |
| | | CCGCCTCAAGCTGTTGCTCCTCCCGCTCCCAAGCCCACACAAGCCGGCGAAGGCACCC |
| | | TGTCTGAGGCCCTGCTGCAACTGCAATTCGACGACGAGGATCTGGGCGCCCTGCTGGG |
| | | CAACAGCACCGATCCCGCTGTCTTTACCGATCTGGCCAGCGTGGATAATAGCGAGTTC |
| | | CAGCAGCTCCTGAACCAGGGTATCCCTGTGGCGCCGCACACAACGGAGCCCATGCTGA |
| | | TGGAGTACCCTGAGGCCATCACCAGACTCGTGACTGGCGCACAACGCCCTCCTGATCC |
| | | AGCACCAGCGCCGTTAGGCGCACCTGGCCTGCCTAACGGCTTGCTGAGCGGCGACGAG |
| | | GATTTCTCCAGCATCGCCGATATGGATTTCAGCGCACTGCTGAGTCAAATCTCCAGCC |
| | | GGGCCAAGCGGTCCGGATCCGGAGAGGGCAGGGGATCTCTCCTTACTTGTGGGGACGT |
| | | CGAGGAAAACCCTGGACCaATGGCCCTGCCCGTGACAGCCCTGCTCCTGCCTCTGGCC |
| | | CTGTTGCTGCATGCAGCACGGCCTGAGCTGCCCACACAAGGCACATTCAGCAACGTGT |
| | | CCACCAACGTGTCCGAGCTGTGCGGCGGCGCTATCGTGGTGCCCGTGTGCTTGGCCTT |
| | | CCTGCTGACCACCCTGCTGGGCGTGCTGTTCTGCTTCAACAAGCGGGACCTGATTAAG |
| | | AAGCACATCTGGCCCAACGTGCCCGACCCTAGCAAGAGCCACATCGCCCAGTGGAGCC |
| | | CTCACACACCTCCCAGGCACAACTTCAACAGCAAGGACCAGATGTACTCCGACGGCAA |
| | | CTTCACCGACGTGTCCGTGGTGGAGATCGAGGCCAACGACAAGAAGCCCTTCCCTGAG |
| | | GACCTGAAGTCCCTGGACCTGTTCAAGAAGGAGAAGATCAACACCGAGGGACACAGCA |
| | | GCGGCATCGGCGGCTCTAGCTGTATGAGCAGCAGCAGGCCCAGCATCTCCAGCAGCGA |
| | | CGAGAACGAGAGCAGCCAGAACACCAGCAGCACCGTGCAGTACAGCACCGTGGTGCAC |
| | | AGCGGCTACAGGCACCAGGTGCCATCAGTGCAAGTGTTCAGCCGATCCGAGTCCACCC |
| | | AGCCTCTGCTGGACAGCGAGGAGAGGCCCGAGGACCTGCAACTGGTGGACCACGTGGA |
| | | CGGAGGCGACGGCATCCTGCCTAGGCAGCAATACTTCAAGCAGAATTGCAGCCAGCAC |
| | | GAGAGCAGCCCTGACATCAGCCACTTCGAGAGGAGCAAGCAAGTGTCCAGCGTGAACG |
| | | AGGAGGACTTCGTGAGGCTGAAGCAGCAGATCAGCGACCACATCAGCCAGAGCTGCGG |
| | | CAGCGGCCAGATGAAGATGTTCCAGGAAGTGTCCGCCGCCGACGCCTTCGGCCCTGGC |
| | | ACAGAAGGACAAGTGGAGAGGTTCGAAACCGTGGGCATGGAGGCCGCCACCGACGAGG |
| | | GAATGCCCAAGAGCTACCTGCCTCAGACCGTGCGGCAGGGCGGCTACATGCCTCAGTA |
| | | AAGGACGGGTGGCATCCCTGTGACCCCTCCCCAGTGCCTCTCCTGGCCCTGGAAGTTG |
| | | CCACTCCAGTGCCCACCAGCCTTGTCCTAATAAAATTAAGTTGCATCATTTTGTCTGA |
| | | CTAGGTGTCCTTCTATAATATTATGGGGTGGAGGGGGGTGGTATGGAGCAAGGGGCAA |
| | | GTTGGGAAGACAACCTGTAGGGCCTGCGGGGTCTATTGGGAACCAAGCTGGAGTGCAG |
| | | TGGCACAATCTTGGCTCACTGCAATCTCCGCCTCCTGGGTTCAAGCGATTCTCCTGCC |
| | | TCAGCCTCCCGAGTTGTTGGGATTCCAGGCATGCATGACCAGGCTCAGCTAATTTTTG |
| | | TTTTTTTGGTAGAaACGGGGTTTCACCATATTGGCCAGGCTGGTCTCCAACTCCTAAT |
| | | CTCAGGTGATCTACCCACCTTGGCCTCCCAAATTGCTGGGATTACAGGCGTGAACCAC |
| | | TGCTCCCttccctgtccttcCGAGGGCAATCTGGCCCATCAAGTGGCCTTCGCCTCTG |
| | | GGAGTAACAAAAATGCACTTCAAAATAGCTTCTGTAATCAAGCTGCATGGGTGGAGTA |
| | | CTCCCCAGCTGACTCCAGGAAGTTCTCTATCCAAAGCTATTCATTAGGCCAGAGCTGT |
| | | GCAAATAATTAGTCACCCACTTGCTCCATAACCCTCCATGACAGCCCAGGCATTGAGT |
| | | CCAGGTGGGACCATCAAGCCATGCTCTGGTGGCTCATGCATTATCATAGAAATGGGAG |
| | | GCTTTATTTATTTTACTAAAAAGAACAAAAACAACAGACTGCTGTCCTTTAGACAATA |
| | | GGATCACGTCATCTGAGCCCTCTGTGCCCCAGGTGACAAGCCCAGCCCCAAGTTCTCT |
| | | TTCCTCAGCCTCCCCACACATGTTCTGGAGGAGATGGGCCCAGCAGGCTGCTCTGAGG |
| | | CCTGGCCCC |

1. One or more nucleic acid(s), wherein the one or more nucleic acids encode at least one of:
a. a first antibody or antigen-binding domain that specifically binds to Prostate Stem Cell Antigen (PSCA), comprising a first variable heavy (VH) chain sequence comprising three heavy chain CDR sequences, CDR-H1, CDR-H2, and CDR-H3, of the VH sequence set forth in SEQ ID NO: 45, and a variable light (VL) chain sequence comprising three light chain CDR sequences, CDR-L1, CDR-L2, and CDR-L3, of the VL sequence set forth in SEQ ID NO: 46; and/or
b. a second antibody or antigen-binding domain that specifically binds Prostate-Specific Membrane Antigen (PSMA), comprising a second variable heavy (VH) chain sequence comprising three heavy chain CDR sequences, CDR-H1, CDR-H2, and CDR-H3, of the VH sequence set forth in SEQ ID NO: 33, and a variable light (VL) chain sequence comprising three light chain CDR sequences, CDR-L1, CDR-L2, and CDR-L3, of the VL sequence set forth in SEQ ID NO: 34.

2. The one or more nucleic acid(s) of claim 1, wherein:
a. the first variable heavy (VH) chain sequence comprises a CDR-H1 comprising the sequence set forth in SEQ ID NO: 47, a CDR-H2 comprising the sequence set forth in SEQ ID NO: 48, a CDR-H3 comprising the sequence set forth in SEQ ID NO: 49, a CDR-L1 comprising the sequence set forth in SEQ ID NO: 50, a CDR-L2 comprising the sequence set forth in SEQ ID NO: 51, and a CDR-L3 comprising the sequence set forth in SEQ ID NO: 52; and/or
b. the second variable heavy (VH) chain sequence comprises a CDR-H1 comprising the sequence set forth in SEQ ID NO: 35, a CDR-H2 comprising the sequence set forth in SEQ ID NO: 36, a CDR-H3 comprising the sequence set forth in SEQ ID NO: 37, a CDR-L1 comprising the sequence set forth in SEQ ID NO: 38, a CDR-L2 comprising the sequence set forth in SEQ ID NO: 39, and a CDR-L3 comprising the sequence set forth in SEQ ID NO: 40.

3. The one or more nucleic acid(s) of claim 1, wherein the first VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 45 and/or the first VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 46.

4. The nucleic acid(s) of claim 1, wherein the first antibody or antigen-binding domain comprises an scFv, optionally wherein the scFv comprises a sequence at least 80%, 85%, 90%, 91%, 92%, 93%, 94%, 95%, 96%, 97%, 98%, 99%, or 100% identical to the sequence as set forth in SEQ ID NO: 44.

5. The one or more nucleic acid(s) of claim 1, wherein the second VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 33 and/or the second VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 34.

6. The one or more nucleic acid(s) of claim 1, wherein the second antibody or antigen-binding domain comprises an scFv, optionally wherein the scFv comprises a sequence at least 80%, 85%, 90%, 91%, 92%, 93%, 94%, 95%, 96%, 97%, 98%, 99%, or 100% identical to the sequence as set forth in SEQ ID NO: 32.

7. One or more nucleic acid(s), wherein the one or more nucleic acids encode a system comprising:
a. a first chimeric polypeptide comprising a priming receptor comprising a first extracellular antigen-binding domain that specifically binds Prostate Stem Cell Antigen (PSCA), wherein the first extracellular antigen-binding domain comprises a first variable heavy (VH) chain sequence comprising three heavy chain CDR sequences, CDR-H1, CDR-H2, and CDR-H3, of the VH sequences set forth in SEQ ID NO: 45, and a light chain comprising a first variable light (VL) chain sequence comprising three light chain CDR sequences, CDR-L1, CDR-L2, and CDR-L3, of the VL sequences set forth in SEQ ID NO: 46, optionally wherein:
i. CDR-H1 comprises the sequence set forth in SEQ ID NO: 47, CDR-H2 comprises the sequence set forth in SEQ ID NO: 48, CDR-H3 comprises the sequence set forth in SEQ ID NO: 49, CDR-L1 comprises the sequence set forth in SEQ ID NO: 50, CDR-L2 comprises the sequence set forth in SEQ ID NO: 51, and CDR-L3 comprises the sequence set forth in SEQ ID NO: 52; and
b. a second chimeric polypeptide comprising a chimeric antigen receptor (CAR) comprising a second extracellular antigen-binding domain that specifically binds to Prostate-Specific Membrane Antigen (PSMA).

8. The nucleic acid(s) of claim 7, wherein the first VH chain sequence comprises a VH sequence at least 80%, 85%, 90%, 91%, 92%, 93%, 94%, 95%, 96%, 97%, 98%, 99%, or 100% identical to the sequence as set forth in SEQ ID NO: 45 and/or the first VL chain sequence comprises a VL sequence at least 80%, 85%, 90%, 91%, 92%, 93%, 94%, 95%, 96%, 97%, 98%, 99%, or 100% identical to the sequence as set forth in SEQ ID NO: 46.

9. The nucleic acid(s) of claim 7, wherein the first extracellular antigen-binding domain comprises a sequence at least 80%, 85%, 90%, 91%, 92%, 93%, 94%, 95%, 96%, 97%, 98%, 99%, or 100% identical to the sequence as set forth in SEQ ID NO: 44.

10. The nucleic acid(s) of claim 7, wherein the second extracellular antigen-binding domain comprises a heavy chain comprising a second variable heavy (VH) chain sequence comprising three heavy chain CDR sequences, CDR-H1, CDR-H2, and CDR-H3, of the VH sequences set forth in SEQ ID NO: 33, and a light chain comprising a second variable light (VL) chain sequence comprising three light chain CDR sequences, CDR-L1, CDR-L2, and CDR-L3, of the VL sequence set forth in SEQ ID NO: 34, optionally wherein
a. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

11. The nucleic acid(s) of claim 7, wherein the second VH comprises a sequence at least 80%, 85%, 90%, 91%, 92%, 93%, 94%, 95%, 96%, 97%, 98%, 99%, or 100% identical to the sequence as set forth in SEQ ID NO: 33 and/or the second VL comprises a sequence at least 80%, 85%, 90%, 91%, 92%, 93%, 94%, 95%, 96%, 97%, 98%, 99%, or 100% identical to the sequence as set forth in SEQ ID NO: 34.

12. The nucleic acid(s) of claim 7, wherein the second extracellular domain comprises a sequence at least 80%, 85%, 90%, 91%, 92%, 93%, 94%, 95%, 96%, 97%, 98%, 99%, or 100% identical to the sequence as set forth in SEQ ID NO: 32.

13. The nucleic acid(s) claim 7, wherein
a. the priming receptor comprises, from N-terminus to C-terminus,
i. the first antigen-binding domain;
ii. a first transmembrane domain comprising one or more ligand-inducible proteolytic cleavage sites; and
iii. an intracellular domain comprising a human or humanized transcriptional effector, wherein binding of the first antigen-binding domain to human PSCA results in cleavage at the one or more ligand-inducible proteolytic cleavage sites; and/or
b. the CAR comprises, from N-terminus to C-terminus,
i. a second antigen-binding domain;
ii. a second transmembrane domain;
iii. an intracellular co-stimulatory domain; and
iv. an intracellular activation domain.

14. The nucleic acid(s) claim 7, wherein the priming receptor comprises a sequence at least 80%, 85%, 90%, 91%, 92%, 93%, 94%, 95%, 96%, 97%, 98%, 99%, or 100% identical to the sequence as set forth in SEQ ID NO: 54 and/or the CAR comprises a sequence at least 80%, 85%, 90%, 91%, 92%, 93%, 94%, 95%, 96%, 97%, 98%, 99%, or 100% identical to the sequence as set forth in SEQ ID NO: 42.

15. The nucleic acid(s) of claim 7, further comprising
a. an optional third chimeric polypeptide comprising a synthetic pathway activator (SPA); and
b. at least one nucleic acid sequence complementary to at least 15 nucleotides of each of:
i. a nucleic acid encoding human Fas Cell Surface Death Receptor (FAS) comprising the sequence set forth in SEQ ID NO: 3; and
ii. a nucleic acid encoding human Transforming Growth factor (TGF)-β Receptor 2 (TGFBR2) comprising the sequence set forth in SEQ ID NO: 4.

16. One or more nucleic acid(s), wherein the one or more nucleic acids encode a system comprising:
a. a first chimeric polypeptide comprising a priming receptor comprising a first extracellular antigen-binding domain that specifically binds Prostate Stem Cell Antigen (PSCA); and
b. a second chimeric polypeptide comprising a chimeric antigen receptor (CAR) comprising a second extracellular antigen-binding domain that specifically binds to Prostate-Specific Membrane Antigen (PSMA), wherein the second extracellular antigen-binding domain comprises a heavy chain comprising a second variable heavy (VH) chain sequence comprising three heavy chain CDR sequences, CDR-H1, CDR-H2, and CDR-H3, of the VH sequences set forth in SEQ ID NO: 33, and a light chain comprising a second variable light (VL) chain sequence comprising three light chain CDR sequences, CDR-L1, CDR-L2, and CDR-L3, of the VL sequence set forth in SEQ ID NO: 34, optionally wherein
i. CDR-H1 comprises the sequence set forth in SEQ ID NO: 35, CDR-H2 comprises the sequence set forth in SEQ ID NO: 36, CDR-H3 comprises the sequence set forth in SEQ ID NO: 37, CDR-L1 comprises the sequence set forth in SEQ ID NO: 38, CDR-L2 comprises the sequence set forth in SEQ ID NO: 39, and CDR-L3 comprises the sequence set forth in SEQ ID NO: 40.

17. One or more expression vector(s) comprising the one or more nucleic acid(s) of claim 1.

18. One or more expression vector(s) comprising the one or more nucleic acid(s) of claim 7.

19. An antibody or antigen-binding domain encoded by the one or more nucleic acid(s) of claim 1.

20. A system encoded by the one or more nucleic acids of claim 7.

21. A cell or population of cells comprising the one or more nucleic acid(s) of claim 1.

22. A cell or population of cells comprising the one or more nucleic acid(s) of claim 7.

23. A pharmaceutical composition comprising the nucleic acid(s) of claim 1, and a pharmaceutically acceptable excipient.

24. A pharmaceutical composition comprising the cell or population of cells of claim 21, and a pharmaceutically acceptable excipient.

25. A pharmaceutical composition comprising the cell or population of cells of claim 22, and a pharmaceutically acceptable excipient.

26. A method of editing a cell, comprising inserting the nucleic acid(s) of claim 1 into the cell.

27. A method of editing a cell, comprising inserting the nucleic acid(s) of claim 7 into the cell.

28. A method of modulating the activity of a cell or immune cell comprising:
a. obtaining a cell or immune cell comprising one or more nucleic acids of claim 1; and
b. contacting the cell or immune cell with a cell expressing PSCA, wherein binding of the priming receptor to PSCA on the cell induces activation of the priming receptor and expression of the chimeric antigen receptor and wherein binding of the chimeric antigen receptor to PSMA on a target cell expressing PSMA modulates the activity of the immune cell.

29. A method of treating a disease or increasing an immune response in a subject comprising administering to the subject a cell comprising the one or more nucleic acids of claim 1.

30. A method of treating a disease or increasing an immune response in a subject comprising administering to the subject a cell comprising the one or more nucleic acids encoding the system of claim 7.


SEQUENCE LISTING (66 sequences)

SEQ ID NO: 1 - protein, 114 aa, Homo sapiens
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 2 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 3 - RNA, 3696 nt, Homo sapiens
  source 1..3696: mol_type = mRNA
ctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 4 - DNA, 4530 bp, synthetic construct
  source 1..4530: mol_type = other DNA
actcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 5 - RNA, 20 nt, synthetic construct
  source 1..20: mol_type = other RNA
gagccatgcttggcttacga

SEQ ID NO: 6 - DNA, 228 bp, synthetic construct
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 135 bp, synthetic construct
  source 1..135: mol_type = other DNA
gttaataattaacatatatgttaatcattaacatatagttaattattaaccgctatgttaatgattaacaacggttaataattaacatatatgttaatcattaacatatatctagagggtatataatgggggcca

SEQ ID NO: 8 - protein, 21 aa, synthetic construct
MALPVTALLLPLALLLHAARP

SEQ ID NO: 9 - protein, 45 aa, synthetic construct
TTTPAPRPPTPAPTIASQPLSLRPEACRPAAGGAVHTRGLDFACD

SEQ ID NO: 10 - protein, 24 aa, synthetic construct
IYIWAPLAGTCGVLLLSLVITLYC

SEQ ID NO: 11 - protein, 42 aa, synthetic construct
KRGRKKLLYIFKQPFMRPVQTTQEEDGCSCRFPEEEEGGCEL

SEQ ID NO: 12 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 14 - DNA, 132 bp, synthetic construct
  source 1..132: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctgg

SEQ ID NO: 15 - DNA, 374 bp, synthetic construct
  source 1..374: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctag

SEQ ID NO: 16 - DNA, 22 bp, synthetic construct
  source 1..22: mol_type = other DNA
ttaagaatcttttcaaacacta

SEQ ID NO: 17 - DNA, 22 bp, synthetic construct
  source 1..22: mol_type = other DNA
ttatgtaaaagacaaacaatgc

SEQ ID NO: 18 - DNA, 22 bp, synthetic construct
  source 1..22: mol_type = other DNA
ttgattaacaagtttcctatgg

SEQ ID NO: 19 - DNA, 22 bp, synthetic construct
  source 1..22: mol_type = other DNA
taaaaatcaatctcatttcctg

SEQ ID NO: 20 - RNA, 919 nt, synthetic construct
  source 1..919: mol_type = other RNA
ccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccataggaaacttgttaatcattagtgaagccacagatgtattgattaacaagtttcctatggtgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagta

SEQ ID NO: 21 - protein, 27 aa, synthetic construct
TTTPAPRPPTPAPTIASQPLSLRPEAC

SEQ ID NO: 22 - protein, 22 aa, synthetic construct
FMYVAAAAFVLLFFVGCGVLLS

SEQ ID NO: 23 - protein, 43 aa, synthetic construct
RKRRRQHGQLWFPEGFKVSEASKKKRREPLGEDSVGLKPLKNA

SEQ ID NO: 24 - protein, 283 aa, synthetic construct
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAM

SEQ ID NO: 25 - protein, 191 aa, synthetic construct
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 26 - protein, 476 aa, synthetic construct
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQIS

SEQ ID NO: 27 - DNA, 48 bp, synthetic construct
  source 1..48: mol_type = other DNA
gagctgcccacacaaggcacattcagcaacgtgtccaccaacgtgtcc

SEQ ID NO: 28 - DNA, 15 bp, synthetic construct
  source 1..15: mol_type = other DNA
gagctgtgcggcggc

SEQ ID NO: 29 - DNA, 66 bp, synthetic construct
  source 1..66: mol_type = other DNA
gctatcgtggtgcccgtgtgcttggccttcctgctgaccaccctgctgggcgtgctgttctgcttc

SEQ ID NO: 30 - DNA, 831 bp, synthetic construct
  source 1..831: mol_type = other DNA
aacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcag

SEQ ID NO: 31 - DNA, 479 bp, synthetic construct
  source 1..479: mol_type = other DNA
cgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 32 - protein, 244 aa, synthetic construct
QVQLVESGGGVVQPGRSLRLSCAASGFIFSSYGMHWVRQAPGKGLEWVTIIWYDGSNRYYADSVKGRFTISRDNSKNTLYLQMNSLRAEDTAVYYCARASGDFYNYFMDVWGKGTTVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKLEIK

SEQ ID NO: 33 - protein, 121 aa, synthetic construct
QVQLVESGGGVVQPGRSLRLSCAASGFIFSSYGMHWVRQAPGKGLEWVTIIWYDGSNRYYADSVKGRFTISRDNSKNTLYLQMNSLRAEDTAVYYCARASGDFYNYFMDVWGKGTTVTVSS

SEQ ID NO: 34 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKLEIK

SEQ ID NO: 35 - protein, 5 aa, synthetic construct
SYGMH

SEQ ID NO: 36 - protein, 17 aa, synthetic construct
IIWYDGSNRYYADSVKG

SEQ ID NO: 37 - protein, 12 aa, synthetic construct
ASGDFYNYFMDV

SEQ ID NO: 38 - protein, 11 aa, synthetic construct
RASQSVSSNLA

SEQ ID NO: 39 - protein, 7 aa, synthetic construct
GASTRAT

SEQ ID NO: 40 - protein, 10 aa, synthetic construct
QQYNNWPPYT

SEQ ID NO: 41 - DNA, 732 bp, synthetic construct
  source 1..732: mol_type = other DNA
caggttcagcttgtggaatctggtggtggcgtggttcagcctgggagatctctgcggctgtcatgcgctgcgtctgggttcatcttctcctcatacggaatgcattgggtcagacaggctcccggcaaagggctcgagtgggttacaattatctggtatgatggcagtaatcgctactacgccgattccgtcaaagggcggttcaccatttcaagggacaatagcaagaacactttgtatctccaaatgaactcccttagagcagaggacaccgccgtgtactattgcgcccgcgcaagcggcgacttctacaattattttatggacgtctggggcaagggcactacggtgacagtgtcctccggaggaggaggatctggaggaggaggaagcggtggcggcgggagtgagatagtaatgacccagtcgcctgccactctaagcgtatctcccggcgaacgggctaccttgagttgtagggcaagtcaatcagtgtcctccaatctggcctggtatcagcaaaaaccgggccaggctcctaggctgctgatatacggcgccagcacacgtgcaactgggattccagcgcgatttagcgggagcggttcagggacggagttcaccttaacaatcagtagcctgcaatctgaggattttgccgtgtactattgtcaacagtataacaactggccaccctacacctttggccagggtacaaagctcgaaatcaag

SEQ ID NO: 42 - protein, 470 aa, synthetic construct
QVQLVESGGGVVQPGRSLRLSCAASGFIFSSYGMHWVRQAPGKGLEWVTIIWYDGSNRYYADSVKGRFTISRDNSKNTLYLQMNSLRAEDTAVYYCARASGDFYNYFMDVWGKGTTVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 1410 bp, synthetic construct
  source 1..1410: mol_type = other DNA
caggttcagcttgtggaatctggtggtggcgtggttcagcctgggagatctctgcggctgtcatgcgctgcgtctgggttcatcttctcctcatacggaatgcattgggtcagacaggctcccggcaaagggctcgagtgggttacaattatctggtatgatggcagtaatcgctactacgccgattccgtcaaagggcggttcaccatttcaagggacaatagcaagaacactttgtatctccaaatgaactcccttagagcagaggacaccgccgtgtactattgcgcccgcgcaagcggcgacttctacaattattttatggacgtctggggcaagggcactacggtgacagtgtcctccggaggaggaggatctggaggaggaggaagcggtggcggcgggagtgagatagtaatgacccagtcgcctgccactctaagcgtatctcccggcgaacgggctaccttgagttgtagggcaagtcaatcagtgtcctccaatctggcctggtatcagcaaaaaccgggccaggctcctaggctgctgatatacggcgccagcacacgtgcaactgggattccagcgcgatttagcgggagcggttcagggacggagttcaccttaacaatcagtagcctgcaatctgaggattttgccgtgtactattgtcaacagtataacaactggccaccctacacctttggccagggtacaaagctcgaaatcaaggcggcagcaaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctagg

SEQ ID NO: 44 - protein, 251 aa, synthetic construct
EIVLTQSPDFQSVTPTEKVTITCRASQYIGDSLHWYQQKPDQSPKLLIKYASQSFSGVPSRFSGSGSGTDFTLTINSLEAEDAATYYCHQSSSLPYTFGQGTKLEIKGSTSGSGKPGSGEGSTKGQVQLVESGGGVVQPGGSLRLSCAASGFTFSSYGMHWVRQAPGKGLEWVAAIWFDGINKFYVDSVKGRFTISRDNSKNTLYLQMNSLRAEDTALYYCARKVHLERRRDVYYYYMDVWGKGTTVTVSS

SEQ ID NO: 45 - protein, 126 aa, synthetic construct
QVQLVESGGGVVQPGGSLRLSCAASGFTFSSYGMHWVRQAPGKGLEWVAAIWFDGINKFYVDSVKGRFTISRDNSKNTLYLQMNSLRAEDTALYYCARKVHLERRRDVYYYYMDVWGKGTTVTVSS

SEQ ID NO: 46 - protein, 107 aa, synthetic construct
EIVLTQSPDFQSVTPTEKVTITCRASQYIGDSLHWYQQKPDQSPKLLIKYASQSFSGVPSRFSGSGSGTDFTLTINSLEAEDAATYYCHQSSSLPYTFGQGTKLEIK

SEQ ID NO: 47 - protein, 5 aa, synthetic construct
SYGMH

SEQ ID NO: 48 - protein, 17 aa, synthetic construct
AIWFDGINKFYVDSVKG

SEQ ID NO: 49 - protein, 17 aa, synthetic construct
KVHLERRRDVYYYYMDV

SEQ ID NO: 50 - protein, 11 aa, synthetic construct
RASQYIGDSLH

SEQ ID NO: 51 - protein, 7 aa, synthetic construct
YASQSFS

SEQ ID NO: 52 - protein, 9 aa, synthetic construct
HQSSSLPYT

SEQ ID NO: 53 - DNA, 753 bp, synthetic construct
  source 1..753: mol_type = other DNA
gagatcgtgctgacccagtctccagactttcagagcgtgacacccaccgagaaagtgaccatcacctgtagggcctctcagtacatcggcgacagcctgcattggtaccagcagaagcccgaccagagccctaagctgctcatcaaatacgccagtcagtccttcagcggcgtgccatccagattcagcggcagcggcagcggaaccgacttcaccctgaccatcaacagcctggaggccgaggacgccgccacctactattgccatcagtcctccagcctgccctacaccttcggccagggaaccaagctggaaatcaagggctccaccagcggcagcggcaagcccggcagcggagaaggatctaccaagggacaggtccaactggtggagagcggcggcggcgtggtgcaacctggaggctccttgaggctgagctgtgctgccagcggcttcaccttttctagctacggaatgcattgggtgcggcaggctcctggcaaaggcctggagtgggtggccgctatctggttcgacggcatcaacaagttctacgtggacagcgtgaagggaaggttcaccatcagccgggacaacagcaagaacaccctgtacctccagatgaacagcctgagggccgaggataccgccctgtactattgcgccaggaaagtccacctggagaggaggagggacgtgtactactactacatggacgtgtggggcaagggcaccaccgtgaccgtgtccagc

SEQ ID NO: 54 - protein, 821 aa, synthetic construct
EIVLTQSPDFQSVTPTEKVTITCRASQYIGDSLHWYQQKPDQSPKLLIKYASQSFSGVPSRFSGSGSGTDFTLTINSLEAEDAATYYCHQSSSLPYTFGQGTKLEIKGSTSGSGKPGSGEGSTKGQVQLVESGGGVVQPGGSLRLSCAASGFTFSSYGMHWVRQAPGKGLEWVAAIWFDGINKFYVDSVKGRFTISRDNSKNTLYLQMNSLRAEDTALYYCARKVHLERRRDVYYYYMDVWGKGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 55 - DNA, 2463 bp, synthetic construct
  source 1..2463: mol_type = other DNA
gagatcgtgctgacccagtctccagactttcagagcgtgacacccaccgagaaagtgaccatcacctgtagggcctctcagtacatcggcgacagcctgcattggtaccagcagaagcccgaccagagccctaagctgctcatcaaatacgccagtcagtccttcagcggcgtgccatccagattcagcggcagcggcagcggaaccgacttcaccctgaccatcaacagcctggaggccgaggacgccgccacctactattgccatcagtcctccagcctgccctacaccttcggccagggaaccaagctggaaatcaagggctccaccagcggcagcggcaagcccggcagcggagaaggatctaccaagggacaggtccaactggtggagagcggcggcggcgtggtgcaacctggaggctccttgaggctgagctgtgctgccagcggcttcaccttttctagctacggaatgcattgggtgcggcaggctcctggcaaaggcctggagtgggtggccgctatctggttcgacggcatcaacaagttctacgtggacagcgtgaagggaaggttcaccatcagccgggacaacagcaagaacaccctgtacctccagatgaacagcctgagggccgaggataccgccctgtactattgcgccaggaaagtccacctggagaggaggagggacgtgtactactactacatggacgtgtggggcaagggcaccaccgtgaccgtgtccagcgccaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagc

SEQ ID NO: 56 - protein, 320 aa, synthetic construct
ELPTQGTFSNVSTNVSELCGGAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 57 - DNA, 960 bp, synthetic construct
  source 1..960: mol_type = other DNA
gagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcag

SEQ ID NO: 58 - DNA, 9213 bp, synthetic construct
  source 1..9213: mol_type = other DNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctcaggttcagcttgtggaatctggtggtggcgtggttcagcctgggagatctctgcggctgtcatgcgctgcgtctgggttcatcttctcctcatacggaatgcattgggtcagacaggctcccggcaaagggctcgagtgggttacaattatctggtatgatggcagtaatcgctactacgccgattccgtcaaagggcggttcaccatttcaagggacaatagcaagaacactttgtatctccaaatgaactcccttagagcagaggacaccgccgtgtactattgcgcccgcgcaagcggcgacttctacaattattttatggacgtctggggcaagggcactacggtgacagtgtcctccggaggaggaggatctggaggaggaggaagcggtggcggcgggagtgagatagtaatgacccagtcgcctgccactctaagcgtatctcccggcgaacgggctaccttgagttgtagggcaagtcaatcagtgtcctccaatctggcctggtatcagcaaaaaccgggccaggctcctaggctgctgatatacggcgccagcacacgtgcaactgggattccagcgcgatttagcgggagcggttcagggacggagttcaccttaacaatcagtagcctgcaatctgaggattttgccgtgtactattgtcaacagtataacaactggccaccctacacctttggccagggtacaaagctcgaaatcaaggcggcagcaaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctaggtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccataggaaacttgttaatcattagtgaagccacagatgtattgattaacaagtttcctatggtgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctccagactttcagagcgtgacacccaccgagaaagtgaccatcacctgtagggcctctcagtacatcggcgacagcctgcattggtaccagcagaagcccgaccagagccctaagctgctcatcaaatacgccagtcagtccttcagcggcgtgccatccagattcagcggcagcggcagcggaaccgacttcaccctgaccatcaacagcctggaggccgaggacgccgccacctactattgccatcagtcctccagcctgccctacaccttcggccagggaaccaagctggaaatcaagggctccaccagcggcagcggcaagcccggcagcggagaaggatctaccaagggacaggtccaactggtggagagcggcggcggcgtggtgcaacctggaggctccttgaggctgagctgtgctgccagcggcttcaccttttctagctacggaatgcattgggtgcggcaggctcctggcaaaggcctggagtgggtggccgctatctggttcgacggcatcaacaagttctacgtggacagcgtgaagggaaggttcaccatcagccgggacaacagcaagaacaccctgtacctccagatgaacagcctgagggccgaggataccgccctgtactattgcgccaggaaagtccacctggagaggaggagggacgtgtactactactacatggacgtgtggggcaagggcaccaccgtgaccgtgtccagcgccaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcagtaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctc

SEQ ID NO: 59 - DNA, 9173 bp, synthetic construct
  source 1..9173: mol_type = other DNA
gggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggcttataaggtacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggataagcttgatatcgaattcatcgatgttaataattaacatatatgttaatcattaacatatagttaattattaaccgctatgttaatgattaacaacggttaataattaacatatatgttaatcattaacatataactagtctagagggtatataatgggggccactagtctactaccagagttcatcgctagcgctaccggatccgccaccatggccctgccagtaacggctctgctgctgccacttgctctgctcctccatgcagccaggcctcaggttcagcttgtggaatctggtggtggcgtggttcagcctgggagatctctgcggctgtcatgcgctgcgtctgggttcatcttctcctcatacggaatgcattgggtcagacaggctcccggcaaagggctcgagtgggttacaattatctggtatgatggcagtaatcgctactacgccgattccgtcaaagggcggttcaccatttcaagggacaatagcaagaacactttgtatctccaaatgaactcccttagagcagaggacaccgccgtgtactattgcgcccgcgcaagcggcgacttctacaattattttatggacgtctggggcaagggcactacggtgacagtgtcctccggaggaggaggatctggaggaggaggaagcggtggcggcgggagtgagatagtaatgacccagtcgcctgccactctaagcgtatctcccggcgaacgggctaccttgagttgtagggcaagtcaatcagtgtcctccaatctggcctggtatcagcaaaaaccgggccaggctcctaggctgctgatatacggcgccagcacacgtgcaactgggattccagcgcgatttagcgggagcggttcagggacggagttcaccttaacaatcagtagcctgcaatctgaggattttgccgtgtactattgtcaacagtataacaactggccaccctacacctttggccagggtacaaagctcgaaatcaaggcggcagcaaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctaggtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggatccttgacttgcggccaacttgtttattgcagcttataatggttacaaataaagcaatagcatcacaaatttcacaaataaagcatttttttcactgcattctagttgtggtttgtccaaactcatcaatgtatcttatcatgtctgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccataggaaacttgttaatcattagtgaagccacagatgtattgattaacaagtttcctatggtgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccatggccttaccagtgaccgccttgctcctgccgctggccttgctgctccacgccgccaggcctgagatcgtgctgacccagtctccagactttcagagcgtgacacccaccgagaaagtgaccatcacctgtagggcctctcagtacatcggcgacagcctgcattggtaccagcagaagcccgaccagagccctaagctgctcatcaaatacgccagtcagtccttcagcggcgtgccatccagattcagcggcagcggcagcggaaccgacttcaccctgaccatcaacagcctggaggccgaggacgccgccacctactattgccatcagtcctccagcctgccctacaccttcggccagggaaccaagctggaaatcaagggctccaccagcggcagcggcaagcccggcagcggagaaggatctaccaagggacaggtccaactggtggagagcggcggcggcgtggtgcaacctggaggctccttgaggctgagctgtgctgccagcggcttcaccttttctagctacggaatgcattgggtgcggcaggctcctggcaaaggcctggagtgggtggccgctatctggttcgacggcatcaacaagttctacgtggacagcgtgaagggaaggttcaccatcagccgggacaacagcaagaacaccctgtacctccagatgaacagcctgagggccgaggataccgccctgtactattgcgccaggaaagtccacctggagaggaggagggacgtgtactactactacatggacgtgtggggcaagggcaccaccgtgaccgtgtccagcgccaccacaacacccgctcccagacctcccacaccagcaccaacaatagcaagtcaacccttgagcttgcgccctgaggcctgcttcatgtacgtggccgccgccgccttcgtgttgctgttcttcgtgggctgtggcgtgctgttgtccagaaagcgccgccgccagcacggacagctctggttccctgagggcttcaaagtgtcagaggccagcaagaagaagcggcgcgaacctctgggcgaggacagcgtgggcctgaagcctctgaaaaacgctatggtgtcaaagctcagccagctccagacagagctgctggccgccctgctggaaagcggcctgagcaaggaggccctgttgcaggctctgggagagccaggcccttacctgctggctggcgagggacctttggataagggcgagagctgcggaggcggacgcggagagctggccgaactgcctaatggcttgggcgagacacgcggcagcgaggacgagacagacgacgacggcgaggatttcacacctcccatcctcaaggagttggagaatctgtctcccgaggaggccgcccaccagaaggccgtggtggagacactgctgcaagaggacccttggcgcgtggccaagatggtgaagagctacctgcaacagcacaacatccctcagcgcgaggtggtggataccaccggcctgaaccagagccacctgtcccagcacctgaacaagggcacacccatgaagacccagaagcgcgccgccctctacacctggtacgtgcgcaagcagcgcgaggttgctcaacagttcacccacgccggccagggcggcttgattgaggaacccacaggcgacgagctgcccaccaagaagggccgccgcaaccgcttcaaatggggacccgcctcgcagcagatactgttccaagcctacgagcgccagaagaatcccagcaaggaggagcgcgagacactcgtggaggagtgcaaccgcgctgagtgcatccagaggggcgtgagccctagccaagcccagggcttgggcagcaacctggtgacagaagtgcgcgtgtataactggttcgccaaccgccgcaaggaggaggccttccgccacaagctggccatgacttgccgcgacgagttccctacaatggtctttcccagcggccagatcagccaggcctctgccctggcaccagcaccaccacaagtgttacctcaagctcccgctcccgctcccgctcctgctatggtgtcggcattggctcaagctcctgctcccgtgccggtgttggcacctggaccgcctcaagctgttgctcctcccgctcccaagcccacacaagccggcgaaggcaccctgtctgaggccctgctgcaactgcaattcgacgacgaggatctgggcgccctgctgggcaacagcaccgatcccgctgtctttaccgatctggccagcgtggataatagcgagttccagcagctcctgaaccagggtatccctgtggcgccgcacacaacggagcccatgctgatggagtaccctgaggccatcaccagactcgtgactggcgcacaacgccctcctgatccagcaccagcgccgttaggcgcacctggcctgcctaacggcttgctgagcggcgacgaggatttctccagcatcgccgatatggatttcagcgcactgctgagtcaaatctccagccgggccaagcggtccggatccggagagggcaggggatctctccttacttgtggggacgtcgaggaaaaccctggaccaatggccctgcccgtgacagccctgctcctgcctctggccctgttgctgcatgcagcacggcctgagctgcccacacaaggcacattcagcaacgtgtccaccaacgtgtccgagctgtgcggcggcgctatcgtggtgcccgtgtgcttggccttcctgctgaccaccctgctgggcgtgctgttctgcttcaacaagcgggacctgattaagaagcacatctggcccaacgtgcccgaccctagcaagagccacatcgcccagtggagccctcacacacctcccaggcacaacttcaacagcaaggaccagatgtactccgacggcaacttcaccgacgtgtccgtggtggagatcgaggccaacgacaagaagcccttccctgaggacctgaagtccctggacctgttcaagaaggagaagatcaacaccgagggacacagcagcggcatcggcggctctagctgtatgagcagcagcaggcccagcatctccagcagcgacgagaacgagagcagccagaacaccagcagcaccgtgcagtacagcaccgtggtgcacagcggctacaggcaccaggtgccatcagtgcaagtgttcagccgatccgagtccacccagcctctgctggacagcgaggagaggcccgaggacctgcaactggtggaccacgtggacggaggcgacggcatcctgcctaggcagcaatacttcaagcagaattgcagccagcacgagagcagccctgacatcagccacttcgagaggagcaagcaagtgtccagcgtgaacgaggaggacttcgtgaggctgaagcagcagatcagcgaccacatcagccagagctgcggcagcggccagatgaagatgttccaggaagtgtccgccgccgacgccttcggccctggcacagaaggacaagtggagaggttcgaaaccgtgggcatggaggccgccaccgacgagggaatgcccaagagctacctgcctcagaccgtgcggcagggcggctacatgcctcagtaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttccgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccc

SEQ ID NO: 60 - protein, 7 aa, synthetic construct
ENLYTQS

SEQ ID NO: 61 - protein, 5 aa, synthetic construct
DDDDK

SEQ ID NO: 62 - protein, 4 aa, synthetic construct
LVPR

SEQ ID NO: 63 - protein, 4 aa, synthetic construct
CPPC

SEQ ID NO: 64 - RNA, 20 nt, synthetic construct
  source 1..20: mol_type = other RNA
atacacgcacacttgcagaa

SEQ ID NO: 65 - protein, 5 aa, synthetic construct
GGGGS

SEQ ID NO: 66 - protein, 18 aa, synthetic construct
GSTSGSGKPGSGEGSTKG